SEQUENCE LISTING (96 sequences)

SEQ ID NO: 1 - DNA, 2654 bp, Homo sapiens
acgcgtccgcctctagctcgagcagcaggagcagcccgcaccggacaacttgcgagccatggggctggcggatgcgtcgggaccgagggacacacaggcactgctgtctgcaacacaagcaatggacctgcggaggcgagactaccacatggaacggccgctgctgaaccaggagcatttggaggagctggggcgctggggctcagcacctaggacccaccagtggcggacctggttgcagtgctcccgtgctcgggcctatgcccttctgctccaacacctcccggttttggtctggttaccccggtatcctgtgcgtgactggctcctgggtgacctgttatccggcctgagtgtggccatcatgcagcttccgcagggcttggcctacgccctcctggctggattgccccccgtgtttggcctctatagctccttctaccctgtcttcatctacttcctgtttggcacttcccggcacatctccgtggggacctttgctgtcatgtctgtgatggtgggcagtgtgacagaatccctggccccgcaggccttgaacgactccatgatcaatgagacagccagagatgctgcccgggtacaggtggcctccacactcagtgtcctggttggcctcttccaggtggggctgggcctgatccacttcggcttcgtggtcacctacctgtcagaacctcttgtccgaggctataccacagctgcagctgtgcaggtcttcgtctcacagctcaagtatgtgtttggcctccatctgagcagccactctgggccactgtccctcatctatacagtgctggaggtctgctggaagctgccccagagcaaggttggcaccgtggtcactgcagctgtggctggggtggtgctcgtggtggtgaagctgttgaatgacaagctgcagcagcagctgcccatgccgatacccggggagctgctcacgctcatcggggccacaggcatctcctatggcatgggtctaaagcacagatttgaggtagatgtcgtgggcaacatccctgcagggctggtgcccccagtggcccccaacacccagctgttctcaaagctcgtgggcagcgccttcaccatcgctgtggttgggtttgccattgccatctcactggggaagatcttcgccctgaggcacggctaccgggtggacagcaaccaggagctggtggccctgggcctcagtaaccttatcggaggcatcttccagtgcttccccgtgagttgctctatgtctcggagcctggtacaggagagcaccgggggcaactcgcaggttgctggagccatctcttcccttttcatcctcctcatcattgtcaaacttggggaactcttccatgacctgcccaaggcggtcctggcagccatcatcattgtgaacctgaagggcatgctgaggcagctcagcgacatgcgctccctctggaaggccaatcgggcggatctgcttatctggctggtgaccttcacggccaccatcttgctgaacctggaccttggcttggtggttgcggtcatcttctccctgctgctcgtggtggtccggacacagatgccccactactctgtcctggggcaggtgccagacacggatatttacagagatgtggcagagtactcagaggccaaggaagtccggggggtgaaggtcttccgctcctcggccaccgtgtactttgccaatgctgagttctacagtgatgcgctgaagcagaggtgtggtgtggatgtcgacttcctcatctcccagaagaagaaactgctcaagaagcaggagcagctgaagctgaagcaactgcagaaagaggagaagcttcggaaacaggctgcctcccccaagggcgcctcagtttccattaatgtcaacaccagccttgaagacatgaggagcaacaacgttgaggactgcaagatgatggtgagctcaggagataagatggaagatgcaacagccaatggtcaagaagactccaaggccccagatgggtccacactgaaggccctgggcctgcctcagccagacttccacagcctcatcctggacctgggtgccctctcctttgtggacactgtgtgcctcaagagcctgaagaatattttccatgacttccgggagattgaggtggaggtgtacatggcggcctgccacagccctgtggtcagccagcttgaggctgggcacttcttcgatgcatccatcaccaagaagcatctctttgcctctgtccatgatgctgtcacctttgccctccaacacccgaggcctgtccccgacagccctgtttcggtcaccagactctgaacatgctacatcctgcccaagactgcacctctggaggtgcagggcacccttgagaagcccctcacccctaggccgcctccaggtgctacccaggagtcccctccatgtacacacacacaactcagggaaggaggtcctgggactccaagttcagcgctccaggtctgggacagggcctgcatgcagtcaggctggcagtggcgcggtacagggagggaactggtgcatattttagcctcaggaataaagatttgtctgcaaaaaaaaaaaaaaaaaaaaaaaaaaaaaaaaaaaaaaaaaaaaaaaaaaaaaaaaaaaa

SEQ ID NO: 2 - protein, 758 aa, Homo sapiens
MGLADASGPRDTQALLSATQAMDLRRRDYHMERPLLNQEHLEELGRWGSAPRTHQWRTWLQCSRARAYALLLQHLPVLVWLPRYPVRDWLLGDLLSGLSVAIMQLPQGLAYALLAGLPPVFGLYSSFYPVFIYFLFGTSRHISVGTFAVMSVMVGSVTESLAPQALNDSMINETARDAARVQVASTLSVLVGLFQVGLGLIHFGFVVTYLSEPLVRGYTTAAAVQVFVSQLKYVFGLHLSSHSGPLSLIYTVLEVCWKLPQSKVGTVVTAAVAGVVLVVVKLLNDKLQQQLPMPIPGELLTLIGATGISYGMGLKHRFEVDVVGNIPAGLVPPVAPNTQLFSKLVGSAFTIAVVGFAIAISLGKIFALRHGYRVDSNQELVALGLSNLIGGIFQCFPVSCSMSRSLVQESTGGNSQVAGAISSLFILLIIVKLGELFHDLPKAVLAAIIIVNLKGMLRQLSDMRSLWKANRADLLIWLVTFTATILLNLDLGLVVAVIFSLLLVVVRTQMPHYSVLGQVPDTDIYRDVAEYSEAKEVRGVKVFRSSATVYFANAEFYSDALKQRCGVDVDFLISQKKKLLKKQEQLKLKQLQKEEKLRKQAASPKGASVSINVNTSLEDMRSNNVEDCKMMVSSGDKMEDATANGQEDSKAPDGSTLKALGLPQPDFHSLILDLGALSFVDTVCLKSLKNIFHDFREIEVEVYMAACHSPVVSQLEAGHFFDASITKKHLFASVHDAVTFALQHPRPVPDSPVSVTRL

SEQ ID NO: 3 - DNA, 2748 bp, Homo sapiens
  CDS (252)..(2468)
aaagaggctttctcaccagccaaagttcaggtactgcccctgtctgagtttggacatgtggcccacagatgccagctgggggctggaatactgtaccttgatcttagggcctggaaaaatcaggtaggtaactctctggtcctaggtggaggtgtaggggaggttgggccagggatgccttcactgtgtctctctggtcttgccaccccagaccgagggacacacaggcactgctgtctgcaacacaagcaatggacctgcggaggcgagactaccacatggaacggccgctgctgaaccaggagcatttggaggagctggggcgctggggctcagcacctaggacccaccagtggcggacctggttgcagtgctcccgtgctcgggcctatgcccttctgctccaacacctcccggttttggtctggttaccccggtatcctgtgcgtgactggctcctgggtgacctgttatccggcctgagtgtggccatcatgcagcttccgcagggcttggcctacgccctcctggctggattgccccccgtgtttggcctctatagctccttctaccctgtcttcatctacttcctgtttggcacttcccggcacatctccgtggggacctttgctgtcatgtctgtgatggtgggcagtgtgacagaatccctggccccgcaggccttgaacgactccatgatcaatgagacagccagagatgctgcccgggtacaggtggcctccacactcagtgtcctggttggcctcttccaggtggggctgggcctgatccacttcggcttcgtggtcacctacctgtcagaacctcttgtccgaggctataccacagctgcagctgtgcaggtcttcgtctcacagctcaagtatgtgtttggcctccatctgagcagccactctgggccactgtccctcatctatacagtgctggaggtctgctggaagctgccccagagcaaggttggcaccgtggtcactgcagctgtggctggggtggtgctcgtggtggtgaagctgttgaatgacaagctgcagcagcagctgcccatgccgatacccggggagctgctcacgctcatcggggccacaggcatctcctatggcatgggtctaaagcacagatttgaggtagatgtcgtgggcaacatccctgcagggctggtgcccccagtggcccccaacacccagctgttctcaaagctcgtgggcagcgccttcaccatcgctgtggttgggtttgccattgccatctcactggggaagatcttcgccctgaggcacggctaccgggtggacagcaaccaggagctggtggccctgggcctcagtaaccttatcggaggcatcttccagtgcttccccgtgagttgctctatgtctcggagcctggtacaggagagcaccgggggcaactcgcaggttgctggagccatctcttcccttttcatcctcctcatcattgtcaaacttggggaactcttccatgacctgcccaaggcggtcctggcagccatcatcattgtgaacctgaagggcatgctgaggcagctcagcgacatgcgctccctctggaaggccaatcgggcggatctgcttatctggctggtgaccttcacggccaccatcttgctgaacctggaccttggcttggtggttgcggtcatcttctccctgctgctcgtggtggtccggacacagatgccccactactctgtcctggggcaggtgccagacacggatatttacagagatgtggcagagtactcagaggccaaggaagtccggggggtgaaggtcttccgctcctcggccaccgtgtactttgccaatgctgagttctacagtgatgcgctgaagcagaggtgtggtgtggatgtcgacttcctcatctcccagaagaagaaactgctcaagaagcaggagcagctgaagctgaagcaactgcagaaagaggagaagcttcggaaacaggctgcctcccccaagggcgcctcagtttccattaatgtcaacaccagccttgaagacatgaggagcaacaacgttgaggactgcaagatgatgcaggtgagctcaggagataagatggaagatgcaacagccaatggtcaagaagactccaaggccccagatgggtccacactgaaggccctgggcctgcctcagccagacttccacagcctcatcctggacctgggtgccctctcctttgtggacactgtgtgcctcaagagcctgaagaatattttccatgacttccgggagattgaggtggaggtgtacatggcggcctgccacagccctgtggtcagccagcttgaggctgggcacttcttcgatgcatccatcaccaagaagcatctctttgcctctgtccatgatgctgtcacctttgccctccaacacccgaggcctgtccccgacagccctgtttcggtcaccagactctgaacatgctacatcctgcccaagactgcacctctggaggtgcagggcacccttgagaagcccctcacccctaggccgcctccaggtgctacccaggagtcccctccatgtacacacacacaactcagggaaggaggtcctgggactccaagttcagcgctccaggtctgggacagggcctgcatgcagtcaggctggcagtggcgcggtacagggagggaactggtgcatattttagcctcaggaataaagatttgtctgcaaaaaaaaaaaaaaaaaaaaa

SEQ ID NO: 4 - protein, 738 aa, Homo sapiens
MDLRRRDYHMERPLLNQEHLEELGRWGSAPRTHQWRTWLQCSRARAYALLLQHLPVLVWLPRYPVRDWLLGDLLSGLSVAIMQLPQGLAYALLAGLPPVFGLYSSFYPVFIYFLFGTSRHISVGTFAVMSVMVGSVTESLAPQALNDSMINETARDAARVQVASTLSVLVGLFQVGLGLIHFGFVVTYLSEPLVRGYTTAAAVQVFVSQLKYVFGLHLSSHSGPLSLIYTVLEVCWKLPQSKVGTVVTAAVAGVVLVVVKLLNDKLQQQLPMPIPGELLTLIGATGISYGMGLKHRFEVDVVGNIPAGLVPPVAPNTQLFSKLVGSAFTIAVVGFAIAISLGKIFALRHGYRVDSNQELVALGLSNLIGGIFQCFPVSCSMSRSLVQESTGGNSQVAGAISSLFILLIIVKLGELFHDLPKAVLAAIIIVNLKGMLRQLSDMRSLWKANRADLLIWLVTFTATILLNLDLGLVVAVIFSLLLVVVRTQMPHYSVLGQVPDTDIYRDVAEYSEAKEVRGVKVFRSSATVYFANAEFYSDALKQRCGVDVDFLISQKKKLLKKQEQLKLKQLQKEEKLRKQAASPKGASVSINVNTSLEDMRSNNVEDCKMMQVSSGDKMEDATANGQEDSKAPDGSTLKALGLPQPDFHSLILDLGALSFVDTVCLKSLKNIFHDFREIEVEVYMAACHSPVVSQLEAGHFFDASITKKHLFASVHDAVTFALQHPRPVPDSPVSVTRL

SEQ ID NO: 5 - DNA, 2612 bp, Mus musculus
aagttctgggcctggcgccaccttctagccggtagtggcctcctgggggagctgtttgaaaagcggtacacgagttaccctctgagggccatgggacttcccgatgggtccgaccaagggacacatcagacgcaggcactcctgtctgcagcccaagaaatggagctgcagaggagagactaccatgtggaaaggccattgctgaaccaggagcagctagaagatctggggcattggggcccagcggcgaagacccaccagtggcgaacttggttccgatgctcccgtgctcgggcccattctcttctgctccagcacgttccagtcctgggctggttaccccggtatcctgtgcgtgaatggctcctgggtgatctgttatctggcctgagtgtggccatcatgcagcttcctcagggcttggcctatgcccttctggcgggattgcctcctatgtttggtctgtacagttctttctaccccgtcttcatctacttcctgtttggtacctccagacacatctctgtggggacctttgctgtaatgtctgtgatggtgggcagtgtgacagaatctctgacagcagataaggccttcgtgcaaggcttgaatgccacagctgatgatgcacgcgtgcaggtggcctacacactcagcttcctggtcggcctcttccaggtggggctgggcctggtacacttcggcttcgttgtcacctacctgtcagagcctctggtccgcagctataccacggctgcatctgtgcaagtcctcgtctctcagctcaagtatgtgtttggcatcaaactcagcagtcactctgggccactgtccgttatctatacagtgctggaggtctgtgcccagctccctgagactgtgcccggcaccgtggtcacggcgattgtggcaggagtggccttggtactggtgaagctacttaatgagaagctgcatcggcgtctgcccctgcccatccctggggaactactcacgctcattggggccactggtatttcctatggtgtgaagctgaatgacagattcaaggtcgatgtggtgggcaacatcaccacagggctcatacccccggtggcacccaagacagagctgtttgcaacgcttgtgggaaatgcctttgccattgctgtggtgggcttcgccattgccatctcactggggaagatctttgccctgaggcatggctaccgtgtggacagtaaccaggagctggtagcccttggcctcagtaacctcattggaggcttcttccagtgcttccccgtgagctgctccatgtctcggagcttggtacaggagagcacgggaggcaacacacaggttgctggagctgtatcttccctttttatccttcttattatcgtcaaacttggggaactcttccgagacctgcccaaggccgtcctggctgctgtcattattgtgaacctaaagggcatgatgaagcagttctcagacatctgctctctttggaaggcaaacagagtggacctgctaatctggctggtgacctttgtggccacaatcctgctgaacctggacattggcctggcagtttccatagtcttctccttgctgctcgtggtcgtccgaatgcagctgccccattactccgtcctggggcaggtgccagatacggatatttatagagacgtggcagaatactctggggccaaggaggtcccgggtgtgaaagtcttccgttcctcagccacgctgtacttcgccaatgctgagctctacagcgactctctgaaagagaagtgcggtgtagatgttgaccccctcatcacccagaagaagaaacgaatcaaaaagcaggagatgaagttaaagcgaatgaagaaagccaagaagtcccagaaacaggatgcttcttccaagatctcctcagtttccgtcaacgtcaacaccaacttggaagacgtcaaaagcaatgacgtcgagggctctgaggccaaggtgcaccaaggggaggagcttcaggatgtagtctccagcaatcaagaagatgccaaggccccaaccatgacctcactgaagtccctgggcctgcctcagccaggcttccatagcctcatcctggacctgagcaccctctcctttgtggacactgtgtgcattaagagcctgaagaatattttccgtgacttccgggagattgaagtggaagtgtacatcgcagcctgttacagtcctgtggtcgcccagcttgaagctggacacttctttgatgaatctatcactaagcagcatgtctttgcctctgtccatgacgctgtgacctttgccctcagccaccggaagtctgtccctaagagccctgttttggccaccaaactctgactttgctgcaggagactgcctacctctggaggttgtgacaggtcaccctcatgaaactccctgcctctgggccacctccaggtgccacctgggactccattccatgtacctgcacatacaattcatggatggatgccctgggactcccaagttcagtgctacaggcctggggcagggtattgcagtcaggctggccttggctgggtgctgaaaaggagctgatgtgggaatacagctttgggaat

SEQ ID NO: 6 - protein, 758 aa, Mus musculus
MGLPDGSDQGTHQTQALLSAAQEMELQRRDYHVERPLLNQEQLEDLGHWGPAAKTHQWRTWFRCSRARAHSLLLQHVPVLGWLPRYPVREWLLGDLLSGLSVAIMQLPQGLAYALLAGLPPMFGLYSSFYPVFIYFLFGTSRHISVGTFAVMSVMVGSVTESLTADKAFVQGLNATADDARVQVAYTLSFLVGLFQVGLGLVHFGFVVTYLSEPLVRSYTTAASVQVLVSQLKYVFGIKLSSHSGPLSVIYTVLEVCAQLPETVPGTVVTAIVAGVALVLVKLLNEKLHRRLPLPIPGELLTLIGATGISYGVKLNDRFKVDVVGNITTGLIPPVAPKTELFATLVGNAFAIAVVGFAIAISLGKIFALRHGYRVDSNQELVALGLSNLIGGFFQCFPVSCSMSRSLVQESTGGNTQVAGAVSSLFILLIIVKLGELFRDLPKAVLAAVIIVNLKGMMKQFSDICSLWKANRVDLLIWLVTFVATILLNLDIGLAVSIVFSLLLVVVRMQLPHYSVLGQVPDTDIYRDVAEYSGAKEVPGVKVFRSSATLYFANAELYSDSLKEKCGVDVDPLITQKKKRIKKQEMKLKRMKKAKKSQKQDASSKISSVSVNVNTNLEDVKSNDVEGSEAKVHQGEELQDVVSSNQEDAKAPTMTSLKSLGLPQPGFHSLILDLSTLSFVDTVCIKSLKNIFRDFREIEVEVYIAACYSPVVAQLEAGHFFDESITKQHVFASVHDAVTFALSHRKSVPKSPVLATKL

SEQ ID NO: 7 - DNA, 2750 bp, Mus musculus
aagttctgggcctggcgccaccttctagccggtagtggcctcctgggggagctgtttgaaaagcggtacacgagttaccctctgagggccatgggacttcccgatgggtccgacttgagagatcttgaaggaggggagaggggccgctgagatctcctggagcccgcggtgcagagaatctctgaagatcttccttgcgtctgcactgctccctcctccattggatacccaggttagagagtcttcccagaccaagggacacatcagacgcaggcactcctgtctgcagcccaagaaatggagctgcagaggagagactaccatgtggaaaggccattgctgaaccaggagcagctagaagatctggggcattggggcccagcggcgaagacccaccagtggcgaacttggttccgatgctcccgtgctcgggcccattctcttctgctccagcacgttccagtcctgggctggttaccccggtatcctgtgcgtgaatggctcctgggtgatctgttatctggcctgagtgtggccatcatgcagcttcctcagggcttggcctatgcccttctggcgggattgcctcctatgtttggtctgtacagttctttctaccccgtcttcatctacttcctgtttggtacctccagacacatctctgtggggacctttgctgtaatgtctgtgatggtgggcagtgtgacagaatctctgacagcagataaggccttcgtgcaaggcttgaatgccacagctgatgatgcacgcgtgcaggtggcctacacactcagcttcctggtcggcctcttccaggtggggctgggcctggtacacttcggcttcgttgtcacctacctgtcagagcctctggtccgcagctataccacggctgcatctgtgcaagtcctcgtctctcagctcaagtatgtgtttggcatcaaactcagcagtcactctgggccactgtccgttatctatacagtgctggaggtctgtgcccagctccctgagactgtgcccggcaccgtggtcacggcgattgtggcaggagtggccttggtactggtgaagctacttaatgagaagctgcatcggcgtctgcccctgcccatccctggggaactactcacgctcattggggccactggtatttcctatggtgtgaagctgaatgacagattcaaggtcgatgtggtgggcaacatcaccacagggctcatacccccggtggcacccaagacagagctgtttgcaacgcttgtgggaaatgcctttgccattgctgtggtgggcttcgccattgccatctcactggggaagatctttgccctgaggcatggctaccgtgtggacagtaaccaggagctggtagcccttggcctcagtaacctcattggaggcttcttccagtgcttccccgtgagctgctccatgtctcggagcttggtacaggagagcacgggaggcaacacacaggttgctggagctgtatcttccctttttatccttcttattatcgtcaaacttggggaactcttccgagacctgcccaaggccgtcctggctgctgtcattattgtgaacctaaagggcatgatgaagcagttctcagacatctgctctctttggaaggcaaacagagtggacctgctaatctggctggtgacctttgtggccacaatcctgctgaacctggacattggcctggcagtttccatagtcttctccttgctgctcgtggtcgtccgaatgcagctgccccattactccgtcctggggcaggtgccagatacggatatttatagagacgtggcagaatactctggggccaaggaggtcccgggtgtgaaagtcttccgttcctcagccacgctgtacttcgccaatgctgagctctacagcgactctctgaaagagaagtgcggtgtagatgttgaccccctcatcacccagaagaagaaacgaatcaaaaagcaggagatgaagttaaagcgaatgaagaaagccaagaagtcccagaaacaggatgcttcttccaagatctcctcagtttccgtcaacgtcaacaccaacttggaagacgtcaaaagcaatgacgtcgagggctctgaggccaaggtgcaccaaggggaggagcttcaggatgtagtctccagcaatcaagaagatgccaaggccccaaccatgacctcactgaagtccctgggcctgcctcagccaggcttccatagcctcatcctggacctgagcaccctctcctttgtggacactgtgtgcattaagagcctgaagaatattttccgtgacttccgggagattgaagtggaagtgtacatcgcagcctgttacagtcctgtggtcgcccagcttgaagctggacacttctttgatgaatctatcactaagcagcatgtctttgcctctgtccatgacgctgtgacctttgccctcagccaccggaagtctgtccctaagagccctgttttggccaccaaactctgactttgctgcaggagactgcctacctctggaggttgtgacaggtcaccctcatgaaactccctgcctctgggccacctccaggtgccacctgggactccattccatgtacctgcacatacaattcatggatggatgccctgggactcccaagttcagtgctacaggcctggggcagggtattgcagtcaggctggccttggctgggtgctgaaaaggagctgatgtgggaatacagctttgggaat

SEQ ID NO: 8 - protein, 735 aa, Mus musculus
MELQRRDYHVERPLLNQEQLEDLGHWGPAAKTHQWRTWFRCSRARAHSLLLQHVPVLGWLPRYPVREWLLGDLLSGLSVAIMQLPQGLAYALLAGLPPMFGLYSSFYPVFIYFLFGTSRHISVGTFAVMSVMVGSVTESLTADKAFVQGLNATADDARVQVAYTLSFLVGLFQVGLGLVHFGFVVTYLSEPLVRSYTTAASVQVLVSQLKYVFGIKLSSHSGPLSVIYTVLEVCAQLPETVPGTVVTAIVAGVALVLVKLLNEKLHRRLPLPIPGELLTLIGATGISYGVKLNDRFKVDVVGNITTGLIPPVAPKTELFATLVGNAFAIAVVGFAIAISLGKIFALRHGYRVDSNQELVALGLSNLIGGFFQCFPVSCSMSRSLVQESTGGNTQVAGAVSSLFILLIIVKLGELFRDLPKAVLAAVIIVNLKGMMKQFSDICSLWKANRVDLLIWLVTFVATILLNLDIGLAVSIVFSLLLVVVRMQLPHYSVLGQVPDTDIYRDVAEYSGAKEVPGVKVFRSSATLYFANAELYSDSLKEKCGVDVDPLITQKKKRIKKQEMKLKRMKKAKKSQKQDASSKISSVSVNVNTNLEDVKSNDVEGSEAKVHQGEELQDVVSSNQEDAKAPTMTSLKSLGLPQPGFHSLILDLSTLSFVDTVCIKSLKNIFRDFREIEVEVYIAACYSPVVAQLEAGHFFDESITKQHVFASVHDAVTFALSHRKSVPKSPVLATKL

SEQ ID NO: 9 - DNA, 2871 bp, Mus musculus
  misc_feature (2850)..(2850): n is a, c, g, or t
gtacttgagcctctgagaagagattcagcacttggcataatgggcacactggttccacagcacactagtgcctgataagccaaagagcctggtgtgacaggatggatgcttctcctgagccccaacagaagggtgggacactggtactggtccgacggcagccccctgtgtcccagggcttgctggaaacactgaaggccaggctgaagaagagctgcacctgcagtatgccatgtgctcaggctctggtgcagggtctgtttcctgccatacactggctgccccagtaccgccttaaggaatacctagcaggtgatgtcatgtcaggattggtcattggcattatcctggtgccacaggccatagcctactcactgctggctgggttacaacccatctatagcctctacacttccttctttgccaacctgatctacttcctcatgggcacctcccgccatgttaatgtgggcatcttcagcctgctgtgtctcatggtgggtcaggtggtggacagagaactccagttggctggctttgacccttcccaggattctctagggcccaagaacaatgacagcaccctcaacaactcagctacaacactgatcattgggctacaggactgcagacgggactgctatgccattcgggttgccactgcccttactctgatggccgggctttatcaggtcctcatgggtatcctccggctgggcttcgtgtctacctatctctcacaaccactgctcgatggctttgctatgggagcttctgtgaccatcttgacttctcaggcaaaacacatgctgggtgtgcagatccctcggcaccagggcctaggcatggtggtccacacatggttgagcttgctgcagaatgtgggacaggccaacatatgtgatgtggtcaccagtgccctgtgcctaggagtgctgctggcagctaaggaactctcagatcgctaccgacaccgtctgaaggtgccaatacccacagagttatttgttattgtggtggccacaattgtgtcccattttggacagctccatacacggtttgactcgagggtggctggcaacattcccactggttttgtggccccacaggtaccagaccctaagataatgtggcgtgtggccctggatgccatgtccctggccctcgtgggctcagccttctccatctccttggcagaaatgtttgcccgtagtcatggctactctgtcagtgccaaccaagagctgctagctgtgggctgctgcaatgtgctgcctgccttcttccactgttttgccactagtgctgctctgtccaaaactctggtgaagatagccactggctgccagacccagttgtccagtgtggtcagtgccgctgtggtgttgctggtgctgctggtgctggctccactgtttcatgatctgcagcgatgtgtgctagcttgtatcattgttgtcagcctgcggggggcgctgcgcaaggtgaaggatctcccacaactttggcggctaagccctgccgatgcactggtctgggtggctactgcagctacctgtgtgctagtcagcaccgaggctggactattagctggagtgttcttctccctgcttagcctggcaggccgtacacagcgtccacgggctgcccttcttgcccgaattggagactccaccttctatgaggatgctgctgaatttgagggcctcctgcccccacctgaggtgcgagtgttccgtttcacaggtccactttactacgccaacaaggacttcttccttcggtctctctacagactcacagggttggatgcagggcactcagccaccaggaaggatcagggcccggaggtaggtgtcagtaacaggagtcttgtcgatggcaaggatctgggttcagtgagcagtggggctgggctggtggtacccctggcatttggtttccacacagtggtcattgactgtgcaccactgctgttcctggatgtggctggcatggccacattgaaagacctccgcagagactacagggccttggacatcaccctgcttctggcttgctgcagtccctcggtgagagacacgttgagaaaagggggcttccttggggaagaacagggagctgaaaatgagctgctattccccagtgtgcacagtgctgtggaggctgcatatgcccgccgtgaggagctgctggctgctgactctgccctctagcagggccctcaagagcccacacctgtgttcacaagccagtgctctgtaggagtggtgtgaatgataaagtcattagagatatacccttggaccacctttgccctggagaagccagggaactccaactaaggaaaggaaagtgcagcacccttaatacattcaaggattccaaaccaagggttcagtgatctagacactctacctctgagtccactgctgccccctggtgcctgttcaaccagggtagctgtacccacacacatgatccctcagcccacacagtgcccagtttgatatactatgttgtcatcttatctgaaacagaatcctgcaaattatatgacctccaagatgccaaaaggacactttcccattccctgtaccaccaggtaccagatatgagctggatatatggccaagcctcaagggtctgaatttctgaaaggcctcctcatgccaggtgctcatcttgactggacccttgcaaaggcagccacctgctccagagccacagtccagtgtcactttctaaggcaatgtgacttatataacctcaacctgaccttggggcacaatgtctctatacagcttatactggtaaccacaatgtggtatatgtatggtacaaagccanacacagtacaccctaacccca

SEQ ID NO: 10 - protein, 704 aa, Mus musculus
MDASPEPQQKGGTLVLVRRQPPVSQGLLETLKARLKKSCTCSMPCAQALVQGLFPAIHWLPQYRLKEYLAGDVMSGLVIGIILVPQAIAYSLLAGLQPIYSLYTSFFANLIYFLMGTSRHVNVGIFSLLCLMVGQVVDRELQLAGFDPSQDSLGPKNNDSTLNNSATTLIIGLQDCRRDCYAIRVATALTLMAGLYQVLMGILRLGFVSTYLSQPLLDGFAMGASVTILTSQAKHMLGVQIPRHQGLGMVVHTWLSLLQNVGQANICDVVTSALCLGVLLAAKELSDRYRHRLKVPIPTELFVIVVATIVSHFGQLHTRFDSRVAGNIPTGFVAPQVPDPKIMWRVALDAMSLALVGSAFSISLAEMFARSHGYSVSANQELLAVGCCNVLPAFFHCFATSAALSKTLVKIATGCQTQLSSVVSAAVVLLVLLVLAPLFHDLQRCVLACIIVVSLRGALRKVKDLPQLWRLSPADALVWVATAATCVLVSTEAGLLAGVFFSLLSLAGRTQRPRAALLARIGDSTFYEDAAEFEGLLPPPEVRVFRFTGPLYYANKDFFLRSLYRLTGLDAGHSATRKDQGPEVGVSNRSLVDGKDLGSVSSGAGLVVPLAFGFHTVVIDCAPLLFLDVAGMATLKDLRRDYRALDITLLLACCSPSVRDTLRKGGFLGEEQGAENELLFPSVHSAVEAAYARREELLAADSAL

SEQ ID NO: 11 - DNA, 3367 bp, Mus musculus
gtcttgcgggaggacgcgccgcgggagcccgtgtacggcggcccggagcgttaaggcgtaaaatttctgctttaaagacatcatcttcaggattccatcaattccaggacatgtgctgtaattcctgtctttgctcctccgtaagtgatgcgtggctgtcttcagaagtgccctctgagcagcagtgattggaagtgaaccatctgtctcctgaaatgtcttcagaaaataaagagcagcatgacctctcaccgagggacttacctgaagaagcctttggtttcccatctgagctccccctggagactcaaagaaggtcaggcactgacttgaggcagtctgagaccggtcatggccggagagctttccgtaggatccacatggagctccgtgagaaaccagacacagacatcaaacagtttgtcatcagagagctccagaagagttgccagtgcagcgcagccaaagtcagagacggggcttttgatttctttcctgttttgcggtggctcccaaaatatgacctgaagaaaaacattttaggtgatgtgatgtctggcctgattgtgggtatcctgttggtgccccagtccattgcttactccctgctggctggccaggagcctatatatggtctatatacatcattttttgccagcattatttatttcctgtttggtacatcccgccacatctctgtgggcatttttggaatactgtgccttatgattggtgaggttgttgaccgagaactacataaagcctgccctgacactgatgctacgtcatcttcgatagcagtgttttcaagtggatgtgtggttgtaaaccatacattagacggactctgtgataaaagctgctatgcaattaaaattggcagcactgtgaccttcatggctggagtttatcaggtagcaatgggcttctttcaagtgggctttgtctctgtctacctctccgacgccttgctgagcgggtttgtcactggcgcctccttcaccatcctcacgtctcaggccaagtacctccttgggctgagccttcctcggagtcatggtgtcggctcagtcattactacctggatccacatcttcagaaacatccgtaacaccaacatctgtgatctcatcaccagccttttatgtcttctggtcctcgtgccatccaaagaactcaatgagcacttcaaggacaagctcaaggctccaattccggtcgagctcattgtcgttgtggcggccacattggcttctcattttggaaaactaaatgggaattacaattccagtattgctggacacattcccactgggtttatgccacccaaagcaccagactggagcctcattcctaatgtggctgtagatgcaatagctatttctatcattggttttgctatcactgtatcactttctgagatgtttgccaagaaacatggctacacggtcaaagcaaaccaagaaatgtatgccattggcttttgcaatatcataccttccttcttccactgcataactactagtgcagctcttgcaaagacattggttaaagagtcaacaggctgccagacgcagctgtcagctatagtgacagcccttgtccttttgttggtccttctggtaatagctcctttattctattcccttcaaaaatgtgtccttggtgtgatcactattgtaaatctccggggcgcacttctgaaatttagagacctgccaaagatgtggaggctaagcagaatggatacagttatctggtttgtgactatgctgtcctccgctctgttaagcactgaaataggcctgctggttggggtttgtttttcaatgttctgtgtgatcctccgtactcagaagccaaagaattcactgcttggtttagaagaagaatctgaaacctttgaatccatttccacttacaagaaccttcggagcaagtccggcatcaaggttttccgcttcatagcccctctctactacataaacaaagagtgctttaaatcagctttgtacaagaaagctcttaacccagtcttggtaaaggcagcttggaaaaaggcagcaaagagaaaactcaaagaagaaatggtgacctttcgtggggacccggatgaagtttcaatgcagctttcccatgatcccttggaggtgcacactatcgtgatcgactgtagtgccatacagtttttagacacagcaggaatccacacactgaaagaagttcgccgggattatgaagccgttgggatccaggttttactggcgcagtgcaatccttctgtgagggattccttggccagaggagaatattgcaaaaaggaagaggaaacccttctcttctacagtctgtctgaagcagtagcctttgcagaagactctcagaatcagaaaggggtgtgtgttgtcaacggtctgagtctctctggtgactgagaaagttaatctgcatcttcctggtaactgggcaagttaataaaaggaagaatagtgtcttgcctgtttcaatgtggaacgttttctactgtgaaaggagaaagccatgcacacttgaaatagttctttgaagctagcctttcgatagtctcatgagcttttggttgggtagaatcaaaaagaagtaaatgttatggcttccggcttatgcagatagcattcttggggtacagtataaatggaatggaactgtaaagcagcccccaacttaagtgccttactttaggaactatatatctgaactctgtccttcaggatgcagagcgaggtaacgccgggctttgcctgacacacactgcagctctagagaggggacgaagaaaggaggcagcgctgctggtacagaggctggaaaagcacaagggcctacggaccctgctgctgtgtctctgttaggattttgctccccaaacagtaagatctcttgctgatagttagaaatcatcatttttcctttccttcatgcaagaaccgtgccatagtctcataagagaatctgacctcacaaccagcctgatactattgtatagaacccatattgtaaatatctaatgtcttaagtcttccctttttatttagaataagtgggtaaagaaagagttttaaaaccagaatggcagtggaagtctttgtctctcttcatctctgtctttgtctttgagaacttgtctgctgttacgccctgtctcacctttgacttattcttgttcccccccccccccccccgggaatccttgctgagttggcatctctgaacaccagtggcagagttgtcagagaaagcagagtattggctcagcagcttcctaacagcactgtgaatgggcttgtggtgcacattcaaggagagatcgtaaaaaaaaaaaaaagaa

SEQ ID NO: 12 - protein, 739 aa, Mus musculus
MSSENKEQHDLSPRDLPEEAFGFPSELPLETQRRSGTDLRQSETGHGRRAFRRIHMELREKPDTDIKQFVIRELQKSCQCSAAKVRDGAFDFFPVLRWLPKYDLKKNILGDVMSGLIVGILLVPQSIAYSLLAGQEPIYGLYTSFFASIIYFLFGTSRHISVGIFGILCLMIGEVVDRELHKACPDTDATSSSIAVFSSGCVVVNHTLDGLCDKSCYAIKIGSTVTFMAGVYQVAMGFFQVGFVSVYLSDALLSGFVTGASFTILTSQAKYLLGLSLPRSHGVGSVITTWIHIFRNIRNTNICDLITSLLCLLVLVPSKELNEHFKDKLKAPIPVELIVVVAATLASHFGKLNGNYNSSIAGHIPTGFMPPKAPDWSLIPNVAVDAIAISIIGFAITVSLSEMFAKKHGYTVKANQEMYAIGFCNIIPSFFHCITTSAALAKTLVKESTGCQTQLSAIVTALVLLLVLLVIAPLFYSLQKCVLGVITIVNLRGALLKFRDLPKMWRLSRMDTVIWFVTMLSSALLSTEIGLLVGVCFSMFCVILRTQKPKNSLLGLEEESETFESISTYKNLRSKSGIKVFRFIAPLYYINKECFKSALYKKALNPVLVKAAWKKAAKRKLKEEMVTFRGDPDEVSMQLSHDPLEVHTIVIDCSAIQFLDTAGIHTLKEVRRDYEAVGIQVLLAQCNPSVRDSLARGEYCKKEEETLLFYSLSEAVAFAEDSQNQKGVCVVNGLSLSGD

SEQ ID NO: 13 - DNA, 1611 bp, Mus musculus
agaaaacacttgttcctctttctgtctcagctgttgagaatactcacccaggatgacctttgtaggactctgagctctcctccctccaaaatagccagttatttttagcccatttcatgggagatttggaccaggttcccatttctttctgaaatatggcctctcaccttgtgactggagcctgaagttggagcgcaggagctgcgccaagagaagccactttcctggtctaggcagtcatatgagggtttccttaatatccagaggctgacatttgtggccctgcccattagtcaccccaaacctaggcgttcccacttcagttcctgcttacccacccacactctggcccttactggtcttttctcacttggatcaggccagagcttccagacctggaaaggatcctgagtgctccatttccagtctaggcagccaggtgtgaaagtgaaaggggggggggggcttaagaagccacccatggtgacaaatacctttgatctcttctttaatcccagcactcactaggcagaggcaggtggatctctgtagacaagtctggtctacagagagttccaggacagccaaggctaaacagagaaaacttgtctggaaaaaaagaaatggaggtggggtggggctggtcttgaggaccactgtgccaggtctcagaaggaaaaatctgagggtgatactcacagcttggtggtccctgagagaccctacaggcctagatctcccacagttgaggctggggcccctggttgcacacctaggcatccagatcggtcaagaatgtctcctctgcataaagtggggaccttctacaaagcatacaattaggtccagccacttaagggcacagaggcataacattggccaagcgtagcgctgggccttcgttccagtcctccagtctcagcagaatgctccagctcccagcaggttaacaagaatcgcaggcagggacagaagctaggccgacgggaaggaaagggttaaaattttattctccgggctcgccccgcccagagagtatgacttggattccacccccagaattctgattggtctgatcttgcacatcctagcggtggagcttgcggccggccaagttctgggcctggcgccaccttctagccggtagtggcctcctgggggagctgtttgaaaagcggtacacgagttaccctctgagggccatgggacttcccgatgggtccggtgagttgcagggcgctgcccaaattccgcgtgcccgcgaagcgctagtagttctgtgactcccggcctgggtcaattcaggaagggaacctccaagtccccgacaggcatttcccgtctgcggagccttagtttaccgccttgccgattttccgaaggaggttatccatcaacagcgagggatgggggcgggctctggcgcgaccccagctcttgagggttgtactgaacgtccctggagtgctaataagggcgctgcagggaagttgctgcttaccgaggagtcctggcacgggaggtgtgccagctgggcgcgttgggactgggagagaaaccgctgcaagacaggctctagccaggcggtggaccagccgcggcctgagtcgg

SEQ ID NO: 14 - DNA, 12 bp, Mus musculus
ggtccggtgagt

SEQ ID NO: 15 - DNA, 12 bp, Mus musculus
aagaagacttga

SEQ ID NO: 16 - DNA, 12 bp, Mus musculus
tcccaggtgagt

SEQ ID NO: 17 - DNA, 12 bp, Mus musculus
ctctagaccaag

SEQ ID NO: 18 - DNA, 12 bp, Mus musculus
gttccggtaatg

SEQ ID NO: 19 - DNA, 12 bp, Mus musculus
tcctagatgctc

SEQ ID NO: 20 - DNA, 12 bp, Mus musculus
ctcagggtgagc

SEQ ID NO: 21 - DNA, 12 bp, Mus musculus
ctgcaggcttgg

SEQ ID NO: 22 - DNA, 12 bp, Mus musculus
ctgtgggtaagt

SEQ ID NO: 23 - DNA, 12 bp, Mus musculus
ctgaagggacct

SEQ ID NO: 24 - DNA, 12 bp, Mus musculus
ttccaggtacga

SEQ ID NO: 25 - DNA, 12 bp, Mus musculus
caacaggtgggg

SEQ ID NO: 26 - DNA, 12 bp, Mus musculus
atctatgtgagt

SEQ ID NO: 27 - DNA, 12 bp, Mus musculus
ccttagacagtg

SEQ ID NO: 28 - DNA, 12 bp, Mus musculus
ctcacggttcgg

SEQ ID NO: 29 - DNA, 12 bp, Mus musculus
ccacagctcatt

SEQ ID NO: 30 - DNA, 12 bp, Mus musculus
cacagggtaagc

SEQ ID NO: 31 - DNA, 12 bp, Mus musculus
tcccaggctcat

SEQ ID NO: 32 - DNA, 12 bp, Mus musculus
aaccaggtctgt

SEQ ID NO: 33 - DNA, 12 bp, Mus musculus
ccacaggagctg

SEQ ID NO: 34 - DNA, 12 bp, Mus musculus
acacaggtaggc

SEQ ID NO: 35 - DNA, 12 bp, Mus musculus
ctgcaggttgct

SEQ ID NO: 36 - DNA, 12 bp, Mus musculus
cccaaggtgagc

SEQ ID NO: 37 - DNA, 12 bp, Mus musculus
ccccaggccgtc

SEQ ID NO: 38 - DNA, 12 bp, Mus musculus
ctcctggtgaga

SEQ ID NO: 39 - DNA, 12 bp, Mus musculus
tatcagctaatc

SEQ ID NO: 40 - DNA, 12 bp, Mus musculus
gcagctgtgagt

SEQ ID NO: 41 - DNA, 12 bp, Mus musculus
cctcaggcccca

SEQ ID NO: 42 - DNA, 12 bp, Mus musculus
tctggggtgagt

SEQ ID NO: 43 - DNA, 12 bp, Mus musculus
ctccaggccaag

SEQ ID NO: 44 - DNA, 12 bp, Mus musculus
gagaaggtgagg

SEQ ID NO: 45 - DNA, 12 bp, Mus musculus
ctctagtgcggt

SEQ ID NO: 46 - DNA, 12 bp, Mus musculus
aaacaggtgggg

SEQ ID NO: 47 - DNA, 12 bp, Mus musculus
tcttaggatgct

SEQ ID NO: 48 - DNA, 12 bp, Mus musculus
gccaaggtgagg

SEQ ID NO: 49 - DNA, 12 bp, Mus musculus
ctgcaggtgcac

SEQ ID NO: 50 - DNA, 12 bp, Mus musculus
aagaatgtaagg

SEQ ID NO: 51 - DNA, 12 bp, Mus musculus
ctgcagattttc

SEQ ID NO: 52 - DNA, 12 bp, Mus musculus
gttacagtgcgt

SEQ ID NO: 53 - DNA, 12 bp, Mus musculus
ccacaggtcctg

SEQ ID NO: 54 - DNA, 12 bp, Mus musculus
gttttggtaagc

SEQ ID NO: 55 - DNA, 12 bp, Mus musculus
acgcaggccacc

SEQ ID NO: 56 - DNA, 12 bp, Mus musculus
ccccaggtaaga

SEQ ID NO: 57 - DNA, 12 bp, Mus musculus
ggctaggacagc

SEQ ID NO: 58 - DNA, 12 bp, Mus musculus
tgcctggtgaga

SEQ ID NO: 59 - DNA, 12 bp, Mus musculus
ttgcagataagc

SEQ ID NO: 60 - DNA, 12 bp, Mus musculus
tatcaggtaaag

SEQ ID NO: 61 - DNA, 12 bp, Mus musculus
ctgcaggtcctc

SEQ ID NO: 62 - DNA, 34 bp, Homo sapiens
acacaggcactgctgtctgcaacacaagcaatgg

SEQ ID NO: 63 - DNA, 38 bp, Homo sapiens
aggatgtagcatgttcagagtctggtgaccgaaacagg

SEQ ID NO: 64 - DNA, 21 bp, Homo sapiens
tacacgagttaccctctgagg

SEQ ID NO: 65 - DNA, 21 bp, Homo sapiens
tacagaccaaacataggaggc

SEQ ID NO: 66 - DNA, 19 bp, Homo sapiens
acaacttgcgagccatggg

SEQ ID NO: 67 - DNA, 21 bp, Homo sapiens
tgttggagcagaagggcatag

SEQ ID NO: 68 - protein, 6 aa, Homo sapiens
TQALLS

SEQ ID NO: 69 - protein, 8 aa, Mus musculus
GTSRHISV

SEQ ID NO: 70 - protein, 98 aa, Mus musculus
GDVMSGLVIGIILVPQAIAYSLLAGLQPIYSLYTSFFANLIYFLNGTSRHVNVGIFSLLCLMVGQVVDRELQLAGFDPSQDSLGPKNNDSTLNNSATT

SEQ ID NO: 71 - protein, 98 aa, Mus musculus
GDVMSGLIVGILLVPQSIAYSLLAGQEPIYGLYTSFFASIIYFLFGTSRHISVGIFGILCLMIGEVVDRELHKACPDTDATSSSIAVFSSGCVVVNHT

SEQ ID NO: 72 - protein, 91 aa, Mus musculus
SDIVSGISTGLVAVLQGLAFALLVNIPPAYGLYAAFFPVITYFFLGTSRHISVGPFPVLSMMVGVVVTRVVSDPNASSELSSSSTENDSFI

SEQ ID NO: 73 - protein, 97 aa, Mus musculus
SDIISGVSTGLVGTLQGMAYALLAAVPVQFGLYSAFFPILTYFVFGTSRHISVGPFPVVSLMVGSVVLSMAPDDHFLVPSGNGSALNSTTLDTGTRD

SEQ ID NO: 74 - protein, 91 aa, Mus musculus
GDLVSGISTGVLQLPQGLAFAMLAAVPPVFGLYSSFYPVIMYCFFGTSRHISIGPFAVISLMIGGVAVRLVPDDIVIPGGVNATNGTEARD

SEQ ID NO: 75 - protein, 85 aa, Mus musculus
GDLLSGLSVAIMQLPQGLAYALLAGLPPMFGLYSSFYPVFIYFLFGTSRHISVGTFAVMSVMVGSVTESLTADKAFVQGLNATAD

SEQ ID NO: 76 - protein, 93 aa, Mus musculus
PDTVSGIMLAVQQVAQGLSFAMLSSVHPVFGLYGSLFPAIIYAIFGMGRHVATGTFALTSLISANAVERLVPQSSRNLTTQSNSSVLGLSEFE

SEQ ID NO: 77 - protein, 97 aa, Mus musculus
GDLLAGLSVGLAQVPQGLILSLLTRQLIPPLNVTYAAFCSSVIYVIFGSCHQMSIGPFFLVSALMINVLKDRPFNNGHLILGTFVKDDFSVPTFYLS

SEQ ID NO: 78 - protein, 94 aa, Mus musculus
PDLLGGLSGGCIQVPQGMAFALLANLPAVNGLYSSFFPLLTYFFLGGIHQMVPGTFAVISILVGNICLQLAPESKFQIFNNVTNETYVDTAAME

SEQ ID NO: 79 - protein, 78 aa, Mus musculus
LDFIAGLSVGLTVIPQALAYAEVAGLPPQYGLYSAFMGCFVYFFLGTSRDVTLGPTAIMSLLVSFYTFREPAYAVLLA

SEQ ID NO: 80 - DNA, 3635 bp, Xenopus laevis
gcacgcggggcacgaggctgggaactgtaattttttcaggaagcatagatggcaacagcaagaatggatatccccagccctggctacaaccaatacctggtgcacagatcagtctactctgaaccaacattccaagaagaaaatgaaagaaaagagcctgttcggaaaactctccaggacagggtgaaaaagaattgcagctgtacatcaaaaaaggctttgtttatagtcaagaagtttctccctattctgaattggttacccaaatacagatggaaagaatggtttttaagtgacctgatctctggagtcagcacaggtcttgttggtaccttgcaaggcctggcatttgcattgcttgctgcagtccctgttggatatggcctctactcttcttttttccctatcttgacttattttttcctgggaacatcaaaacacatttctgtgggaccattcccagtggtcagtttgatggtgggatcagttgttctgtctattgctccagatgaaaaatttgcaatattaggcaactctacaggactgaacaaaacaataatagacacagtagcaagagatgcagccagagttgccgtttcaggaacactcagctttctaattggaattatacagcttgcacttggcgtatttcaaattggctttattatcagatatttggcagatccattagttggtggttttactacagctgcagccttccaagtgtttgtttcacaattcaaactagtgctcaatgtccccacaaagaactacaacggagtactctctatcatatatacaataatagatatctttaccaacattgcgaaaaccaacattgctgatctgattgctggacttctaacatttgttgtttgcgttgtggtgaaagaaataaatgatcgatacaaacgcatctttcgggtacctattcctatagaagtaattgtgaccatagtagctacagggatttcatatggcgtcaatctggaaaagaaatataatgcagggattgtgaagacaattccaactggattcataccacctatgacaccagacgtgagcctcttctcacaaataggtagttcagcattttccatcggtattgttgcctatgctgtagctgtgtctgtgggaaaagtttatgctacaaagcacaactatgaagttgatggaaatcaggagttcattgcctttggaataagcaatctattcggtggagtattttcttgcttttgtgccaccacggccctatcacgtacagctatccaggaaagcactggaggcaaaacacagattgctgggattatctcagctgggattgtgttaattgcaattgtggcacttggcaagctccttgaaccccttcaaaagtcagtgctagcggccattgtcattgccaatctgaaggggatgttttggcaagtttttgatgtacctcgcttatggaaacagaacaaatgggactcagttatctgggtttttacctgcattgcatcaatccttctgggacttgatttaggactgctagctggcttgttgtttggattagtgactattattttacgtgttcagttcccttcatgtggagctcttggtagtgttcctggcacagacatttacaaaaatgtcaaagaatacaaaaatctcatagagccagaaggagttaaaatcattcgctatacaagtggtatgttttatggaaatatcgacgggtttaagaatgccatcaaatcaattgttgggtttgatgcggtgaaagtttataacaagagaacaaaagcactgaggaaaatacaaaagcttattaaaaaaggacaactaagatcaacaaagaatggcattattagcagcacaggagcagataatgaaggttatgagcctgatgatgatcccgaggacccagaacaagaaaataatgaagcagttcaaacaaaagaagtagagatccaagtagactggaactcagaacttccaataaaagtatctgtgcccaaagtctccattcacagcatcatttttgattttgggcaaatccacttcattgatgtggttgcagtgagatctcttaaagtgatatttaaagaatttaagagaattgatgtagagccatacattgcagcatatgaagatggtgttttacaaaaaatggaaaaatgctttttctttgatgaggtgatcaaacgagacatcttctttttgaccgtccatgatgctgtgctgcatatagagaacctgagaaagttctatgatggtcatgatcctcttcttgaaaagatttcactcatgcaagaatctaaggaaccattggagttcaccacaactgacccaacagaccaagaactggatgcccaggaaggggctctccgatcactggcacactgaagaaaggcatctgtaacactcattagggacagtatcatcctttgggttcaataacgagacatttgctgtttcattaacatctactaataccatgctatgcatatgataaaaatgttatgaatgtttatgaattatatgatgggcaactcctcaaaaaccttccaaacagcattttaaatgtgtaaaacagatagacaacaaaaccaagtgtccttatactactggttttaagcatccttcctttctgataaggactgaacagccatttagagattgaggtttaaagtataatgagcctaggtgatattgaagtcaacagatgttatgattgagaggtattccgatttatgcacctaccaggacaagagaggaggaaaggaatatgtgcaacattctaatgaataaacatataaacaaaaataaaattgagtgttgaggcaaagaacatgactgttacccaaacatttaagccacaacatcaaattcttcaaattctgtgaactaagataagtttaatataacaaatacatgttggcctgactgcctcattaaatggcaaaatatttacataaattaagcatattcattttaagtcacaagggggttagttattaaaatccgaatgccaaaatctccaaaaattctagttttttttttttttactataaaatctgaatttttagtgaaaaaaaaaaacaaacaaatttttcaaaatttattataccccgagaatggaaaaagtcaggatacgaaaatccggcattttagacctgtcgaggtggtatatatatatatataagtcaattgcagagtttcctatagtatttggaaattttctcgctgaaattagccataaaattctactattttggactttttgggcaaaaatctgaaaaattcagggtttttgggaaaaacccttaaaaatccagcaatttggggaaaactctgaaaaaaacatacgatttggattttctctagattttgttgagtttgtccccgatccaattaaattgtgtttttttattaataaataaagtcacaacatcagattttttttttggtcagacttttttcatttaaatcagaaaaattctgattttgataaataatgccctaagtgtagcaaatattaatatgtggctgtgaactatagtcagtttattataagtattttacaagtatagatcagtatagctctatagtataaatcttactaaactaaagtaatttagtagtatctgtgaaaa

SEQ ID NO: 81 - protein, 778 aa, Xenopus laevis
MATARMDIPSPGYNQYLVHRSVYSEPTFQEENERKEPVRKTLQDRVKKNCSCTSKKALFIVKKFLPILNWLPKYRWKEWFLSDLISGVSTGLVGTLQGLAFALLAAVPVGYGLYSSFFPILTYFFLGTSKHISVGPFPVVSLMVGSVVLSIAPDEKFAILGNSTGLNKTIIDTVARDAARVAVSGTLSFLIGIIQLALGVFQIGFIIRYLADPLVGGFTTAAAFQVFVSQFKLVLNVPTKNYNGVLSIIYTIIDIFTNIAKTNIADLIAGLLTFVVCVVVKEINDRYKRIFRVPIPIEVIVTIVATGISYGVNLEKKYNAGIVKTIPTGFIPPMTPDVSLFSQIGSSAFSIGIVAYAVAVSVGKVYATKHNYEVDGNQEFIAFGISNLFGGVFSCFCATTALSRTAIQESTGGKTQIAGIISAGIVLIAIVALGKLLEPLQKSVLAAIVIANLKGMFWQVFDVPRLWKQNKWDSVIWVFTCIASILLGLDLGLLAGLLFGLVTIILRVQFPSCGALGSVPGTDIYKNVKEYKNLIEPEGVKIIRYTSGMFYGNIDGFKNAIKSIVGFDAVKVYNKRTKALRKIQKLIKKGQLRSTKNGIISSTGADNEGYEPDDDPEDPEQENNEAVQTKEVEIQVDWNSELPIKVSVPKVSIHSIIFDFGQIHFIDVVAVRSLKVIFKEFKRIDVEPYIAAYEDGVLQKMEKCFFFDEVIKRDIFFLTVHDAVLHIENLRKFYDGHDPLLEKISLMQESKEPLEFTTTDPTDQELDAQEGALRSLAH

SEQ ID NO: 82 - DNA, 2640 bp, Xenopus laevis
ccacgcgtccgcccacgcgtccgctctttggactcatggaatcctcaactgacagcaagtattttgtggcaagatctgtttattcagaccagtccttcaaggaagaacatgagaagagagaaattgttcacagaccacttaaacaaaagttaaaaaaaacattcagctgtacccctaaaaaggcatacagggtagccaaaacttttatacctgtcttggactggcttcccaaatacagatggaaagaatggattgtgtcagatattatagctggagtcagtgtaggtctaatttcagctttacaaggtctggctttcggattgttggctggagttcctattcagtttgggctatattcctctttctttcctgttttaacatactgcttcttggggacctctaagcacatttcagttgggccattcccagtggtatgtctgatggttgggattgttactatatctatggctccagatgatcaattttccgttataaccaatggcactacagtcataaacacaacggccagggatgcagctagaattaacatctgtgggacactctccttcctgattggaatcctacagctttttctgggaatatttcggattggattcattgttaggtacctggcagatccactgatcggaggcttcaccacagctgccgctttccaagtgactgtttcacaaataaaaaccatcctcaatgttcctgccaaaaactacaatggtgtgctttccataatatatacaattatagatatattttccaacattgctcaaactaattttgccgacctcattgctgggctgctgactttagtaattgttctagcggtaaaggaggtaaatgatcgctttaaagagaagattcgtgtgcctattcctatagagattattgtgacaattgtagctacaggaatttcctatggagcgaacctagcagggaaatacaatgcaggaataataaagaccattccgagcggattcattccagctatgtcaccagatgtcagcttgttttcctcgctcattagttctgcgttttccactggaatagttgcatatgctattgtaatatcagttggaaaggtctatgggacaaagaataactacagggttgatggaaatcaggaattcattgcctatgggatcagcaatatgtttggaggcattttctcttgtttttgtgctagcactgctctgtctcgtactgcaattcaagaaggaactggaggaaaaagccagattgcaagcatgatttcagcactcatggttctcattgctattgtggccattggcaggctacttgaacctctgcaaaagtcggtgttggcagctattgttgtagccaatttaaagggaatgttcatgcaggtccgtgatattcctgttctttggagacagaatagatgggatgcagttatatgggttttcacatgcgtctcagcaatcatccttggtttagatttgggattgttggctggtttagtgtttggactattgactgttgttctgcgtgtccagttcccatcatgcagttctcttgcaaatatccataacacagatctttataaagatgtaaagatgtacaaagatcttattgagccaggaggtgtaagaattattcgattttctggaggaattttctatggcaatattgaaggtttgaagaatggcattaaaaaaatagttggatttgatgctaccagagtttttgacaagagggcaaaggctcttcgtatgattcaaaaccttatcaagaaaggtcaactgggagcaacttggaatggtattgtcagtggtacagggatcgataatgagggtttcgaactagatgatgaacaagatgacccagaattagaacaaaaccacgacattcacacaaatgaagtggagatccaagtggattggaattcagagctccccttaacagtgtctgtccctaaagttactgttcacactattgtttttgattttgcaccagtcacctttctcgatgtgatggcagtgaaaaacttaaaactgataattaaagaattcaagaggattgatgtagatatttacattgcagcctgtgatgataatgtctacaacaaaatggaagtctgcggattttttgatgatgctatcaagcctgacatctttttcctcactgtccatgatgctatgctctacatagaaaaggagagaaactttgaaagtgcaagtgatcccatacttgaaaagatttcactaatgcaggagaataaaggacagctggacttcttgatagagccagtagatccagtacaccaggagttagcggcacatgaagaggctattaaacgatacgcatcataaggcaccctcgagctgccaaactggattcaaacaccatcttcttatctctaaccataccagagtctacaaacttcttttcaagtacagagatatatgtcatgtaattaaattatataaatatttgtacaacaaaagaaagaaatgctgcaactgttttactgtatgttattgaaagttagtccttttattgtgtgttctttttttgatctttagtaaatgctatgtacaatgtatactgtatcccattacataaaaataaacgtattctttttaacattcaaaaaaaaaaaaaaaa

SEQ ID NO: 83 - protein, 769 aa, Xenopus laevis
MESSTDSKYFVARSVYSDQSFKEEHEKREIVHRPLKQKLKKTFSCTPKKAYRVAKTFIPVLDWLPKYRWKEWIVSDIIAGVSVGLISALQGLAFGLLAGVPIQFGLYSSFFPVLTYCFLGTSKHISVGPFPVVCLMVGIVTISMAPDDQFSVITNGTTVINTTARDAARINICGTLSFLIGILQLFLGIFRIGFIVRYLADPLIGGFTTAAAFQVTVSQIKTILNVPAKNYNGVLSIIYTIIDIFSNIAQTNFADLIAGLLTLVIVLAVKEVNDRFKEKIRVPIPIEIIVTIVATGISYGANLAGKYNAGIIKTIPSGFIPAMSPDVSLFSSLISSAFSTGIVAYAIVISVGKVYGTKNNYRVDGNQEFIAYGISNMFGGIFSCFCASTALSRTAIQEGTGGKSQIASMISALMVLIAIVAIGRLLEPLQKSVLAAIVVANLKGMFMQVRDIPVLWRQNRWDAVIWVFTCVSAIILGLDLGLLAGLVFGLLTVVLRVQFPSCSSLANIHNTDLYKDVKMYKDLIEPGGVRIIRFSGGIFYGNIEGLKNGIKKIVGFDATRVFDKRAKALRMIQNLIKKGQLGATWNGIVSGTGIDNEGFELDDEQDDPELEQNHDIHTNEVEIQVDWNSELPLTVSVPKVTVHTIVFDFAPVTFLDVMAVKNLKLIIKEFKRIDVDIYIAACDDNVYNKMEVCGFFDDAIKPDIFFLTVHDAMLYIEKERNFESASDPILEKISLMQENKGQLDFLIEPVDPVHQELAAHEEAIKRYAS

SEQ ID NO: 84 - DNA, 2457 bp, Xenopus laevis
ttccaataaatatttgacctccttaatgaatgatcatttcctctcatagatcaagacagatggatatgtctacctatgggaacagccagtatctcctggccagaccaatttacactcaaccaacttttgagaagctgaacatggagaaaacaccaaaaaaaaatactcgcaaacctttcaaaaataggttacagaaaaccttcagctgtacatctacaaaggctgttcatatggtcaagaaatatatccctgtgctgaactggctgccaaaatacagctggaagtcattgtttgtgcacgatttgatttcaggagtcagcacagggatggttggtaccctacaaggtctggcgtttgcgttacttgctgaagttccagtaggatatggaatttattcttctttttttccaattctgacatattttttcctgggaacttctaaacatatttcagttgggccattcccagtgatcagtttaatggtaggctctgttgttctgtccatggctccaaatgaaaattttattgtatacaacaccacaaatctcaatgaaacggaaatgataattgacacagaagccagggatgctgctagagtccttgtatcaggaacattaagtttcttaattggaatcatacagcttgtgttaggagctcttcagtttggctttattgtaagatatttggcagagccattagtgcgaggcttcaccacaggggctgctttccaagcattcatctctcaaatgaaactgatactcaatgttcccacaaatacccacagtggaatgttttcaacattttatacaatgaatgatatcttctccaatatttctaaaacaaatgtggcagacctcattgctggacttctaacaatttttgtatgtgtggcggtcaaggaaattaatgagcggtataaaaattttctgcgcatacccattcctatagagataattgtgtctttaatagctgcatggatttcatatggagtagatctggaaaataaatataatgcaggcacagtaaagaatattccaagtgggttcataccacccatgatgccagatgtgagcatgtttccccaaattattagctctgcaatttccattggaatagtggcatatgctgttgctgtatcattgggaaaggtttttgccaccaaatataactatgcaattgatggaaatcaggaatttgttgcatttggggtcagcaatatattcagtgggtttttctcttgcttttgtgccaccactgctttgtctcgaacagctattcaagaaagtactggaggtaaaactcagattgctggtttgatatctgctgcaactattataattactatgtttgtccttggccagtttcttcaacctctacagaagtctgtactggcagcgattgtcatatccaacttaaaggggatgttttggcaagctctggatatacctcgtttgtggaaacagaataagtgggatgcagctatctggatctttgcatgtttttcaaccatcattttggatcttgatttaggactcttgtgtggattaatgtttggattatttactgtaattctacggatacagttcccctcttgtcactcccttggaaatcttcctggaacagaaatctaccgagatctcaagaagtacaaaaatgtcgtggaacaggaaggaattaaaattattcggttttccagtggtattttctatggaaatgtcgacagcttaaaaaatggcataaaatccattgttggatttgattcagttagagttttcaacaagagagcgaaagcagagctgaagataagtgagcttatcaagaaaggacaattgaaagtaactaagaatggaattataacaaatacattcacggaagattcagaatctgatgaagaatcacaacagtttgaaaatcctcaaaacaaaaaggaagaaacggaattccaaggggactggaattcagatctacctgtattaaattctgttcctaaagtctttattcacagcatcattttggattttggacatgtcaattttcttgatgttgtggcagtgacatcccttaaactgattttaaaagatttcagaagaattgctgtcgatgtttatattgcaggatgtgatgatgaaatttttgagaaactggaagtatgtggcttctttgaggataacaacaaaccagacatctgttttctgtcggtccacgaggctgtaatttacatattaaaccaaagagagttctgtagtgatcaaaattcacttggcaaggcggcagttgtgaaaaatgaacatgttagttcattagaactggatctacaggatgaggagaacagagcatgttccagtgtttccagtataagaagatctggatcttcaggacgattgtctttaggctgaacaaataacctttacaatggattccaaaatgatattaaccctctgtaaaaaaaaaaaaaaa

SEQ ID NO: 85 - protein, 788 aa, Xenopus laevis
MIISSHRSRQMDMSTYGNSQYLLARPIYTQPTFEKLNMEKTPKKNTRKPFKNRLQKTFSCTSTKAVHMVKKYIPVLNWLPKYSWKSLFVHDLISGVSTGMVGTLQGLAFALLAEVPVGYGIYSSFFPILTYFFLGTSKHISVGPFPVISLMVGSVVLSMAPNENFIVYNTTNLNETEMIIDTEARDAARVLVSGTLSFLIGIIQLVLGALQFGFIVRYLAEPLVRGFTTGAAFQAFISQMKLILNVPTNTHSGMFSTFYTMNDIFSNISKTNVADLIAGLLTIFVCVAVKEINERYKNFLRIPIPIEIIVSLIAAWISYGVDLENKYNAGTVKNIPSGFIPPMMPDVSMFPQIISSAISIGIVAYAVAVSLGKVFATKYNYAIDGNQEFVAFGVSNIFSGFFSCFCATTALSRTAIQESTGGKTQIAGLISAATIIITMFVLGQFLQPLQKSVLAAIVISNLKGMFWQALDIPRLWKQNKWDAAIWIFACFSTIILDLDLGLLCGLMFGLFTVILRIQFPSCHSLGNLPGTEIYRDLKKYKNVVEQEGIKIIRFSSGIFYGNVDSLKNGIKSIVGFDSVRVFNKRAKAELKISELIKKGQLKVTKNGIITNTFTEDSESDEESQQFENPQNKKEETEFQGDWNSDLPVLNSVPKVFIHSIILDFGHVNFLDVVAVTSLKLILKDFRRIAVDVYIAGCDDEIFEKLEVCGFFEDNNKPDICFLSVHEAVIYILNQREFCSDQNSLGKAAVVKNEHVSSLELDLQDEENRACSSVSSIRRSGSSGRLSLG

SEQ ID NO: 86 - DNA, 3200 bp, Xenopus laevis
cttgtgttgtaaatcaattcctgaactttgaacacaatctataaaatttattgtataggtaacacagagaaatgttcatgcaactttaactttggcaagtgcattcttcatgccatccaggaagatgaggactgctttttactaagagaaaaacattattttgatgccattttttaagaaaacactaagttgcaatgaaaaacgcagtcaacatgaaatatgaaagagaacaaagcaagatggatgaagattcacagcataccctaattcatctggaaagaaaagctactaacagagttagcctgtgtaagactgtaaaagccaaagttaaaaagcaatgcacctgcaatagcaagcaactcaaaaaaacattcattggctttttcccagtgttacgctggctcccaaagtatgacttcaaagaaaatacatggggtgatgtgatgtcgggacttatcattggtatcatattggtgccccaagctatagcatattctcttctggctgggttgaaacccatttacagcttgtacacatctttttttgctaatattatttatttcttaatggggacctcccgacacgtatcagtgggcattttcagcttaataagtttgatggttggccaggttgttgatcgagaagtacagctggcaggttttgacctagatgatgatgcagttcctcaaataaataacttcaacatgagtgacttaaacattacaagatccataaacattagtatgggcctgatggacattgagtgtggaaaagaatgttatgcaatcagcgttgcagctatactgacattcacggctggtatttatcaggtcattatggggagttttcgcttgggtttcctctctatgtatctatcagagccaatgttggatggatttgcaactggagcttccctgacaatattaactgcccaagtgaaataccttcttggaataaaaatcccacgtagtccaggcattggcatgctggtgacaacttggtataacatatttgcaaatattcaccacagcaactattgtgatattattacaagtgccatttgtattgctgttctggttgcagctaaagaaataggagatcgatacaaagaaaaaattaaaattccacttccaacagaattagtggtaattgtagtggctactgtggtatcacattactgcaatctgaaggaagtttatggatctgctgtctctggtgtaatcccaactggatttatacccccacaagttccaaattttagcctttttggcaaaatagcagtagatgcaataccccttgcagttatcagttttgcctttaccatctctttgtcagaaatgtttgcaaaaaaatatgcatatacagtggaagcaaaccaggagatgtttgccattggtttctgtaatataatcccatcatttttccactgttttgccacgagtgcagctctagcaaaaactctagtgaaaacttccactgggtgcatgacacaagtatctagcgttattagtgcaattgtggtactgctagttcttcttttctttgcacctctgttttattcactgcagaaatgtgtcttagcctgtataataattgttagtttacgaggcgctctaaggaaattcagagatctgccaactctgtggcgcctaaataaaatagatgcagtggtctggtgtgtgacggtggctgctgcagctctggtcagtacagaagtaggcctcatggtaggagtgattttttcaatgttgtgtctaattttacgctctcagttgccttacaccaccatgcttaatcaaattgaagacactgtgttttatgaagattgccaaaaatatgacaaccttcttccaattcccaaagtaaaaatattccgattcaactcacccctccattacgcaaataaaggatattttttaaaggctctgttcaaaatggctgctatggatccaggcctagttaatgcacagcgcaaaaaaatggaaaagaaagctaaaaatcagggaaaacggaaacaagtagatgccgcaaacaaccttggttatggtgaaacccaaatagaacttgttgaaaagcgtaacgatttgcaaaccattattcttgattgttcttgtattgcatttttggacataactggcatgaatgtattaaagggtttgttaaaagactacaaggaagtgcaagtcagtgtgctattggcttgctgcagcacatctgtaattgactctcttattagggggggttatttcggcaaagagaattcagacattcacaaactgcttttttacactgtccatgatgctgtacagtttgcaagagcacaaactatttctgccactgactccactgtgtaaatgatgataaaaattcacaggctttcaaaagcattctgaagtatatattacattgcatttatctggtagttcctgattgtagaggtgttaactattatgttttcttcaggacactgaatttgacagtatcactctaagacacttagtgaaagtgcaaaccacctattttatatagtggtataccacacagcctgtaatctaatactgctgctggagatcttgtcaagtgacagagttggctatgactacagtaaatattgctgtattaatgtaagaaagataaaatgattgtgttatcctgaggcaccataagacaggcactctgcccagctttaatcccatgtctccaccatgtcacttgcccaatcaagtgcctgcaggggttgtcagctccccccttcatagtcttcatagcttatttctcctatggaagcaaccaagttatttttattaataatataatctaaaaagtgccaacatattcaatggctgaatagagctgttgtgaacttttggattatagtagattcaggaagatgtaattgcatacctttattttccccactactttgctaattttcatcccacccagttagaagaattttctggagagaacagtgtgctgactagtgctataagtatcagtctgtagccgtatacctataacaagcataaataattatgtatcaaatggtgaaatgtgggaaaaacgctttaaactgattttcattccaatggaattgcatgtaaaatgatacacgcccttcaatgtcttttgttggaacaatgtttatggcaaaaaacccactcacacttacaccaagatctatcaaaaaaaaaaaaaaa

SEQ ID NO: 87 - protein, 719 aa, Xenopus laevis
MKNAVNMKYEREQSKMDEDSQHTLIHLERKATNRVSLCKTVKAKVKKQCTCNSKQLKKTFIGFFPVLRWLPKYDFKENTWGDVMSGLIIGIILVPQAIAYSLLAGLKPIYSLYTSFFANIIYFLMGTSRHVSVGIFSLISLMVGQVVDREVQLAGFDLDDDAVPQINNFNMSDLNITRSINISMGLMDIECGKECYAISVAAILTFTAGIYQVIMGSFRLGFLSMYLSEPMLDGFATGASLTILTAQVKYLLGIKIPRSPGIGMLVTTWYNIFANIHHSNYCDIITSAICIAVLVAAKEIGDRYKEKIKIPLPTELVVIVVATVVSHYCNLKEVYGSAVSGVIPTGFIPPQVPNFSLFGKIAVDAIPLAVISFAFTISLSEMFAKKYAYTVEANQEMFAIGFCNIIPSFFHCFATSAALAKTLVKTSTGCMTQVSSVISAIVVLLVLLFFAPLFYSLQKCVLACIIIVSLRGALRKFRDLPTLWRLNKIDAVVWCVTVAAAALVSTEVGLMVGVIFSMLCLILRSQLPYTTMLNQIEDTVFYEDCQKYDNLLPIPKVKIFRFNSPLHYANKGYFLKALFKMAAMDPGLVNAQRKKMEKKAKNQGKRKQVDAANNLGYGETQIELVEKRNDLQTIILDCSCIAFLDITGMNVLKGLLKDYKEVQVSVLLACCSTSVIDSLIRGGYFGKENSDIHKLLFYTVHDAVQFARAQTISATDSTV

SEQ ID NO: 88 - DNA, 2829 bp, Xenopus laevis
tctaataaggtgacactatagaaccagattcattccccctccctatagagggctgtgttgttagttccccagcagcagctagtaaggggggttattttctctggcacattgctctctccacgatttaaaggtatctctgcaaggttactgagaagctgaggactatccattcatttggttaagacacacttcttctttcgtcaggattgcccgtactgctggtcactacaccactttgtaatgtttctctaagttctcttccccctcaagtggggagatttatgaggtttacacaagggacacttgtagatctgaaaagagatgggtgtccccagtagaagggaagatctcagtaatgggatagtgatggatcctgcagtaaggcatccagtgctgagtgaggccgagttggaggaaatggcaccaagatctcagcgagcagctccctccacattaaccaggatgaagaagaagataagatgttccggatcagttgcaaagtccctgcttctgaaattcattccaattcttggttggttaccacggtatccagtaaaggaatggctgctcggagacatagtatctggtcttagtgttggcatcattcagctcccacaaggtttagcttatgctttactggctggagttccaccggtgtttggattgtattcctcatttttccctgtacttctctatgccatatttgggacttcaagacatatttcccctggaacctttgctgtgatttctgtgatggttggtagtgtgactgaatcactggtgccgtcagaaaactacaggttacctggaaatgaatcagtgattgatatagcagccagagacaatgacagagttgaggtggcatcggccttgacattcctagttggactttttcagattatgttgggtctggtgcaagttggctttgttgtaacctacctctctgaacccttgatccgaggctacaccagtgctgctgcaatccatgtcactgtgtctcagatgaagagtgttctaggcgtgcagatcagccaaagaagtcatccattgtctctaatttatgcattcgtgaatctctgtgccaaggttcctgagacaaatattgcttcccttctaattgggtgcatctccatcactgttctgtttctggttaagtttctaaacgacaagtacagctcaaagatccgtatgcccataccaatcgaactcatcacgctaattgtagccactgggatatcatatggtgccagtctccaccaagtctatggagtggacattgtaggggagatcccaactgggatgaaagctccaatgttaccaaacacaaatatttttgcaagagtggtcggcaatgcctttgccatcgctgttgtggtctatgcctttaccatctccctggctaagatgtttggtgttaaacatggatacaatattgacagcaatcaggaactgatcgccttggggctgagtaattcaataggaagcttctttcagtgcttcacaattgggacagccatgtcaagaagtctggtgcaagaaagcacaggagggcacagtcaggtagccagtgctgtctcttccttggtcatccttattatcattctaaaagctggggagctgtttgaaaccctaccaaaagccatactggctgcagttgttgtagtcaatctcaaaggtatttacaagcagtttactgatgtgccaatgttatggagatccaacaaatttgatctgttggtgtggctggttacgtttattgctacaattctgctgaacctggatatcggactggctgtttccgtggcattttcactcttaactgtcatcttcagaacccagaaaccccattactccatcctgggaaaggtccacaatactgatatctacagagacgtagcccagtttgatcaggtacaagaaatacaaggagtgaagatcttccagtcatcctgcaccctttactttgccaatgctaatctgtatgcagaggctgttaaaaagatgtgtggcactgatgtggatacattgattgaactgaagaaaaaagcaatgaaaaagcaaaagcagctacaagaaaaagcagagaaacagatgaagaaagaaaacaaaaaaagagaaaaagaattggactccattgtctctaatagtcctgctgccaaagagccagagattcagattgcagcagactatgaagtcttagaggaagctggcctggactaccttggctcggaaaaatgcaacttgcactctcttatcctggacttgagcacagctggctttttggatacagtcagtattaaagttttaaaaaatatatttagggatttccaagaaatcgatgttcaggtttaccttactggatgccaagtatacatcattgaacagttagaggccgccaactttttctccaagtcagtcaccaagaacctgctctttaattctgtccatgatgcagtgacgtatatttccaggacacacggccagcaggatacgaaaggaagtgacacgtgcctaaatacaaagatatgagtcctgccggctgcaacaaggtttttttggctccgtagaaatggaacacgttacaaaacaaagccaaattctcaatatgcttcacttatgtgggtggaaatcaattttgtatgcaagatacttgtattttatttctcaaaagtgttttaaggttcagatatttttatatttttaaaatatacattgaaaaaaccctcaccagccactaatgggacactgaaggggcaatctcctaaaaataggaaaacatttagaaacctgaccatgaataaaggcctttttatataaaaaaaaaaaaaa

SEQ ID NO: 89 - protein, 735 aa, Xenopus laevis
MGVPSRREDLSNGIVMDPAVRHPVLSEAELEEMAPRSQRAAPSTLTRMKKKIRCSGSVAKSLLLKFIPILGWLPRYPVKEWLLGDIVSGLSVGIIQLPQGLAYALLAGVPPVFGLYSSFFPVLLYAIFGTSRHISPGTFAVISVMVGSVTESLVPSENYRLPGNESVIDIAARDNDRVEVASALTFLVGLFQIMLGLVQVGFVVTYLSEPLIRGYTSAAAIHVTVSQMKSVLGVQISQRSHPLSLIYAFVNLCAKVPETNIASLLIGCISITVLFLVKFLNDKYSSKIRMPIPIELITLIVATGISYGASLHQVYGVDIVGEIPTGMKAPMLPNTNIFARVVGNAFAIAVVVYAFTISLAKMFGVKHGYNIDSNQELIALGLSNSIGSFFQCFTIGTAMSRSLVQESTGGHSQVASAVSSLVILIIILKAGELFETLPKAILAAVVVVNLKGIYKQFTDVPMLWRSNKFDLLVWLVTFIATILLNLDIGLAVSVAFSLLTVIFRTQKPHYSILGKVHNTDIYRDVAQFDQVQEIQGVKIFQSSCTLYFANANLYAEAVKKMCGTDVDTLIELKKKAMKKQKQLQEKAEKQMKKENKKREKELDSIVSNSPAAKEPEIQIAADYEVLEEAGLDYLGSEKCNLHSLILDLSTAGFLDTVSIKVLKNIFRDFQEIDVQVYLTGCQVYIIEQLEAANFFSKSVTKNLLFNSVHDAVTYISRTHGQQDTKGSDTCLNTKI

SEQ ID NO: 90 - DNA, 2279 bp, Sus scrofa
ggcttatggggctgtcagaagcgcctggccagagggacacacaagcactgctgtccacaacacaaacaatggagctgaggaggcgagactaccatgttgagcggcctctgctcaaccaagaacagctggaggagctggggaaccgaggctcagcaactgggacctggcagtggcgaaactggtttcgttgctcccgtgctcgggcctacgcccttctgctccagtacctcccagttttgacctggctaccccagtatcctgtgcgtgagtggcttctgggtgacctgttgtctggcctgagtgtggccattatgcagctaccacagggcttggcctatgccctcctagccggactgccccccgtgtttggcctctacagctccttctaccctgtctttatctatttcctgtttggcacttcccggcacatctctgtgggcacctttgctgtcatgtctgtgatggtgggcagtgtgacagaatcgctggccccagatgaggacttcctgcaggcctcgaactctacggttgatgtggcagccagagatgctaggcgggtgcagctggcctctgcactcagtgtcctagtgggcctctttcaggtggggctgggcctggtccactttggcttcgtggtcacctacttgtcagagcctctggtccgtggctataccacagccgcgtccgtgcaggtcttcatctcacagctcaagtatgtgtttggcctccatctgagcagccgctctgggccactgtccctcatctatacagcactggaggtctgctggaatctgcccaagactgtagtcaacaccttggtcactgcagttgtggcagggttggtgctcgtgctggtgaagctattgaacaacaagctgcagaaacatctgcccgtgcctctccccggggagctactcacgctcatcggggccacaggcatctcctacggcataggactgaatgaggtggatgtcgtgggcagaatccctgcagggctggtgcccccagtggcccccagcccgcagctgtttgcacgtcttgtgggaaatgccttcgccatcgccgtggttggtttcgccattgccatctcgctggggaagatcttcgccctgaggcatggctaccgggtggacagcaaccaggagctggtggcgctcggcctcagtaacttcattgggggcatcttccagtgcttccctgtgagctgctctatgtctcggagcctggtacaggagagcaccgggggcaacacacaggtggctggagccatctcctccctcttcatcctcattatcatcctcaaacttggagaactcttccaagacctgcccaaggcagttctggcagccgtcatcattgtgaacctgaagggcatgttgatgcagttcactgacttatgctccctctggaagacaaatcgagtggatctgctcatctggctagtgacctttgtggccaccatcctgctgaacctggaccttggcctggcggttgcgatagccttctccatgctgctcgttgtggtccgcatacagctgccccactactctgtcctggggcagatgccagacacagacgtttacagagatgtggcagagtactcagaagccagggaggtcccgggcgtgaaaatcttccgctcctcaaccaccatgttctttgccaacgctgagctctacggtgacgcgctcaagcagaggtgtggtgtggatgtggaccacctcatctcccagaagaagaagctgctcaggcggcaggagctaaagctgaaacgactgcagaagggcaacaagctcgtgaaaaaggacacttctatttccatcaatgtaaacactggtatcacaaacatcgagagcaacgatgtggagggctccaacgtcaaggtgagtgcagagaatgagctagaggatatagcagcgggagatcaagaagatgccaaggccccagccatgtcctcactgaaggccctgggtctgcctcagccagatttccacagcctcatcctggacctgggcaccctctcctttgtggacactgtgtgtctcaagagcctgaagaatattttccgtgacttccgggagatcgaggtggaggtgtacatggccgcctgccactctcctgtggtctcgcagctcgaggctgggcatttctttgatgcatctatcaccaagcagcatctctttgcttcagtccacgatgctgtcatctttgccctccagcacccacggtctggccccgtcagccctgctttggttactaaactctgaccatgctgccac

SEQ ID NO: 91 - protein, 753 aa, Sus scrofa
MGLSEAPGQRDTQALLSTTQTMELRRRDYHVERPLLNQEQLEELGNRGSATGTWQWRNWFRCSRARAYALLLQYLPVLTWLPQYPVREWLLGDLLSGLSVAIMQLPQGLAYALLAGLPPVFGLYSSFYPVFIYFLFGTSRHISVGTFAVMSVMVGSVTESLAPDEDFLQASNSTVDVAARDARRVQLASALSVLVGLFQVGLGLVHFGFVVTYLSEPLVRGYTTAASVQVFISQLKYVFGLHLSSRSGPLSLIYTALEVCWNLPKTVVNTLVTAVVAGLVLVLVKLLNNKLQKHLPVPLPGELLTLIGATGISYGIGLNEVDVVGRIPAGLVPPVAPSPQLFARLVGNAFAIAVVGFAIAISLGKIFALRHGYRVDSNQELVALGLSNFIGGIFQCFPVSCSMSRSLVQESTGGNTQVAGAISSLFILIIILKLGELFQDLPKAVLAAVIIVNLKGMLMQFTDLCSLWKTNRVDLLIWLVTFVATILLNLDLGLAVAIAFSMLLVVVRIQLPHYSVLGQMPDTDVYRDVAEYSEAREVPGVKIFRSSTTMFFANAELYGDALKQRCGVDVDHLISQKKKLLRRQELKLKRLQKGNKLVKKDTSISINVNTGITNIESNDVEGSNVKVSAENELEDIAAGDQEDAKAPAMSSLKALGLPQPDFHSLILDLGTLSFVDTVCLKSLKNIFRDFREIEVEVYMAACHSPVVSQLEAGHFFDASITKQHLFASVHDAVIFALQHPRSGPVSPALVTKL

SEQ ID NO: 92 - protein, 17 aa, Mus musculus
QEQLEDLGHWGPAAKTH

SEQ ID NO: 93 - protein, 19 aa, Mus musculus
KVHQGEELQDVVSSNQEDA

SEQ ID NO: 94 - protein, 17 aa, Mus musculus
YRLTGLDAGHSATRKDQ

SEQ ID NO: 95 - protein, 17 aa, Homo sapiens
KEQHNVSPRDSAEGNDS

SEQ ID NO: 96 - protein, 739 aa, Homo sapiens
MSSESKEQHNVSPRDSAEGNDSYPSGIHLELQRESSTDFKQFETNDQCRPYHRILIERQEKSDTNFKEFVIKKLQKNCQCSPAKAKNMILGFLPVLQWLPKYDLKKNILGDVMSGLIVGILLVPQSIAYSLLAGQEPVYGLYTSFFASIIYFLLGTSRHISVGIFGVLCLMIGETVDRELQKAGYDNAHSAPSLGMVSNGSTLLNHTSDRICDKSCYAIMVGSTVTFIAGVYQVAMGFFQVGFVSVYLSDALLSGFVTGASFTILTSQAKYLLGLNLPRTNGVGSLITTWIHVFRNIHKTNLCDLITSLLCLLVLLPTKELNEHFKSKLKAPIPIELVVVVAATLASHFGKLHENYNSSIAGHIPTGFMPPKVPEWNLIPSVAVDAIAISIIGFAITVSLSEMFAKKHGYTVKANQEMYAIGFCNIIPSFFHCFTTSAALAKTLVKESTGCHTQLSGVVTALVLLLVLLVIAPLFYSLQKSVLGVITIVNLRGALRKFRDLPKMWSISRMDTVIWFVTMLSSALLSTEIGLLVGVCFSIFCVILRTQKPKSSLLGLVEESEVFESVSAYKNLQTKPGIKIFRFVAPLYYINKECFKSALYKQTVNPILIKVAWKKAAKRKIKEKVVTLGGIQDEMSVQLSHDPLELHTIVIDCSAIQFLDTAGIHTLKEVRRDYEAIGIQVLLAQCNPTVRDSLTNGEYCKKEEENLLFYSVYEAMAFAEVSKNQKGVCVPNGLSLSSD

(19) United States
(12) Patent Application Publication
(10) Pub. No.: US 2006/0014940 A1
(43) Pub. Date: Jan. 19, 2006

(54) Cloning and characterization of slc26a6, slc26a1, and slc26a2 anion exchangers

(76) Inventors: David B. Mount, Boston, MA (US); Michael F. Romero, Cleveland, OH (US)

Correspondence Address: JENKINS, WILSON & TAYLOR, P. A., 3100 TOWER BLVD, SUITE 1400, DURHAM, NC, 27707, US

(21) Appl. No.: 10/505,263

(22) PCT Filed: Feb. 28, 2003
(86) PCT No.: PCT/US03/06469

§ 371 Date: Jul. 29, 2005

Related U.S. Application Data
(60) Provisional application No. 60/360,275, filed on Feb. 28, 2002.

(51) Int. Cl. C07K 14/705; C12P 21/06; C07H 21/04

(52) U.S. Cl. 536/23.1; 530/350; 435/69.1; 435/320.1; 435/325

(57) ABSTRACT

Isolated nucleic acids encoding SLC26A6, SLC26A1, and SLC26A2 anion transporter polypeptides, recombinantly expressed SLC26A6, SLC26A1, and SLC26A2 anion transporter polypeptides, heterologous expression systems for recombinant expression of SLC26A6, SLC26A1, and SLC26A2 anion transporter polypeptides, assay methods employing the same, and methods for modulation of anion transport activity.

### CROSS REFERENCE TO RELATED APPLICATIONS

This application is based on and claims priority to U.S. Provisional Application Ser. No. 60/360,275, filed Feb. 28, 2002, and entitled CLONING AND CHARACTERIZATION OF SLC26A6, SLC26A1, and SLC26A2 ANION EXCHANGERS, herein incorporated by reference in its entirety.

### GRANT STATEMENT

This work was supported by grants RO1 DK57708, PO1 DK038226, RO1 DK56218, and T32 DK07569-12 from the U.S. National Institute of Health. Thus, the U.S. government has certain rights in the invention.

### FIELD OF THE INVENTION

The present invention generally relates to anion transporter polypeptides and anion transport activity mediated by the same. More particularly, the present invention provides isolated nucleic acids encoding SLC26 anion transporter polypeptides, isolated and functional SLC26 anion transporter polypeptides, a heterologous expression system for recombinant expression of SLC26 anion transporter polypeptides, methods for identifying modulators of an anion transporter, and uses thereof.

Table of Abbreviations

| | |
|---|---|
| AE - | anion exchanger |
| ATCC - | American Type Culture Collection |
| BAC - | bacterial artificial chromosome |
| BLAST - | basic alignment and search tool |
| CF - | cystic fibrosis |
| CFTR - | cystic fibrosis transmembrane |
| | conductance regulator |
| cM - | centimorgan |
| CMV - | cytomegalovirus |
| cRNA - | complementary RNA |
| CpG - | unmethylated cytosine-guanine |
| | dinucleotides |
| DIDS - | 4,4′-diisothiocyanatostilbene-2,2′-disulfonic |
| | acid |
| DTST - | diastrophic dysplasia sulphate transporter; |
| | SLC26A2 |
| EGFP - | enhanced green fluorescent protein |
| EST - | expressed sequence tag |
| Fab - | antigen-binding antibody fragment |
| FCS - | Fluorescence Correlation Spectroscopy |
| Fv - | antigen-binding antibody fragment |
| GAPDH - | glyceraldehyde-3-phosphate |
| | dehydrogenase |
| GFP - | green fluorescent protein |
| HEPES - | 4-(2-hydroxyethyl)-1- |
| | piperazineethanesulfonic acid |
| HTGS - | high throughput genomic sequences |
| HUGO - | Human Genome Organization |
| I.M.A.G.E. - | Integrated Molecular Analysis of |
| | Genomes and their Expression |
| | database |
| LA-PCR - | long and accurate PCR |
| LDB - | location database |
| MGD - | Mouse Genome Database |
| MHC - | major histocompatibility complex |
| mSLC26A6 - | mouse SLC26A6 |
| NMDG - | N-methyl-D-glucamine |
| ORF - | open reading frame |
| OSM - | osmolarity |
| PAC - | P-1 derived artificial chromosome |
| pCMV-SLC26 - | construct encoding SLC26 under the |
| | control of a CMV promoter |
| PCR - | polymerase chain reaction |
| PFU - | plaque-forming unit |
| pHᵢ - | intracellular pH |
| PKA - | phosphokinase A |
| PKC - | phosphokinase C |
| RACE - | rapid amplification of cDNA ends |
| RH - | radiation hybrid |
| RT-PCR - | reverse transcription - polymerase chain |
| | reaction |
| Sat-1 - | sulphate anion transporter-1 (SLC26A1) |
| SDS - | sodium dodecyl sulphate |
| SELDI-TOF - | Surface-Enhanced Laser |
| | Desorption/Ionization Time-Of-flight |
| | Spectroscopy |
| SLC26 - | solute carrier 26 protein family |
| Sp1 - | pregnancy-“specific” beta 1-glycoprotein; |
| | Cys₂-His₂ zinc finger transcription |
| | factor |
| SPR - | surface plasmon resonance |
| STAS - | sulfate transporter and anti-sigma domain |
| STS - | sequence-tagged site |
| TESS - | Transcription Element Search Software |
| UTR - | untranslated region |
| Vₘ - | membrane voltage |
| xSLC26A1 - | Xenopus SLC26A1 ortholog |
| xSLC26A6 - | Xenopus SLC26A6 ortholog |
| xPDS1 - | Xenopus SLC26A4 (pendrin or PDS) |
| | ortholog |
| xPDS2 - | Xenopus SLC26A4 (pendrin or PDS) |
| | ortholog |
| xPDS3 - | Xenopus SLC26A4 (pendrin or PDS) |
| | ortholog |

### BACKGROUND OF THE INVENTION

Anion exchange at the plasma membrane is primarily mediated by the products of two structurally distinct gene families: (1) the AE (anion exchanger) genes, which form a subset of the bicarbonate transporter SLC4 superfamily (Romero et al., 2000; Tsuganezawa et al., 2001); and (2) the SLC26 or sulphate permease gene family (Everett & Green, 1999). Members of the SLC26 gene family have been identified by expression cloning (Bissig et al., 1994), subtractive cDNA cloning (Zheng et al., 2000), and positional cloning of human disease genes (Everett & Green, 1999).

The SLC26 gene family has been highly conserved during evolution, and homologues have been identified in bacteria, yeast, plants, and animals. See Everett & Green (1999) Hum Mol Genet 8:1883-1891 and Kere et al. (1999) Am J Physiol 276:G7-G13. Four mammalian SLC26 genes have been described (SLC26A1, SLC26A2, SLC26A3, and SLC26A4). The Drosophila genome contains at least nine family members, suggesting that additional mammalian paralogues also exist.

Physiological roles for individual family members include transepithelial salt transport (Everett & Green, 1999; Scott & Karniski, 2000), thryoidal iodide transport (Scott et al., 1999), development and function of the inner ear (Everett & Green, 1999; Zheng et al., 2000), sulphation of extracellular matrix (Satoh et al., 1998), and renal excretion of bicarbonate (Royaux et al., 2001) and oxalate (Karniski et al., 1998a). The various substrates transported by the SLC26 anion exchangers include sulphate (SO₄²⁻), chloride (Cl⁻), iodide (I⁻), formate, oxalate, hydroxyl ion (OH⁻), and bicarbonate (HCO₃⁻) (Bissig et al., 1994; Karniski et al., 1998a; Satoh et al., 1998; Moseley et al., 1999; Scott & Karniski, 2000; Soleimani et al., 2001).

The multiple physiological roles of SLC26 transporters are supported by diverse anion transport properties. Despite a capacity for versatile anion exchange, SLC26 anion transporters display distinct patterns of anion specificity and cis-inhibition. For example, SLC26A4, also known as pendrin, can transport chloride, hydroxyl ion, bicarbonate, iodide, and formate, but neither oxalate nor sulphate (Scott et al., 1999; Scott & Karniski, 2000; Royaux et al., 2001; Soleimani et al., 2001).

Thus, there exists a long-felt need in the art to identify and functionally characterize SLC26 anion transporters as pharmaceutical targets for diseases and disorders related to abnormal anion transport activity.

To meet this need, the present invention provides functional SLC26A6, SLC26A1, and SLC26A2 anion transporter polypeptides. The present invention also provides methods for identifying and using modulators of anion transport via SLC26A6, SLC26A1, and SLC26A2.

### SUMMARY OF INVENTION

The present invention provides isolated and functional SLC26A6 polypeptides useful in the assays and screening methods disclosed herein. A functional SLC26A6 polypeptide can comprise: (a) a polypeptide encoded by a nucleic acid of any one of odd-numbered SEQ ID NOs:1-7; (b) a polypeptide encoded by a nucleic acid substantially identical to any one of odd-numbered SEQ ID NOs:1-7; (c) a polypeptide comprising an amino acid sequence of any one of even-numbered SEQ ID NOs:2-8; or (d) a polypeptide substantially identical to any one of even-numbered SEQ ID NOs:2-8.

A functional SLC26A6 polypeptide can also comprise a polypeptide encoded by an isolated nucleic acid molecule selected from the group consisting of: (a) an isolated nucleic acid molecule encoding a polypeptide of any one of even-numbered SEQ ID NOs:2-8; (b) an isolated nucleic acid molecule of any one of odd-numbered SEQ ID NOs:1-7; (c) an isolated nucleic acid molecule which hybridizes to a nucleic acid of any one of odd-numbered SEQ ID NOs:1-7 under wash stringency conditions represented by a wash solution having less than about 200 mM salt concentration and a wash temperature of greater than about 45° C., and which encodes a functional SLC26A6 polypeptide; and (d) an isolated nucleic acid molecule differing by at least one functionally equivalent codon from the isolated nucleic acid molecule of one of (a), (b), and (c) above in nucleic acid sequence due to the degeneracy of the genetic code, and which encodes a functional SLC26A6 polypeptide encoded by the isolated nucleic acid of one of (a), (b), and (c) above.

Preferably, a functional property of a SLC26A6 polypeptide of the invention comprises Cl⁻-fomate exchange, Cl⁻—Cl⁻ exchange, SO₄²⁻ exchange, Cl⁻-oxalate exchange, Cl⁻-base exchange, or combinations thereof. Cl⁻-base exchange is preferably electrogenic and can utilize substrates such as HCO₃⁻.

The present invention also provides isolated human SLC26A6a polypeptides, and nucleic acids encoding the same. Preferably, a human SLC26A6a polypeptide comprises: (a) a polypeptide of SEQ ID NO:2; or (b) a polypeptide encoded by a nucleic acid of SEQ ID NO:1.

The present invention further provides isolated mouse SLC26A6 polypeptides, SLC26A1 polypeptides, and nucleic acids encoding the same. Preferably, a mouse SLC26A6 polypeptide comprises: (a) a polypeptide of SEQ ID NO:6 or 8; or (b) a polypeptide encoded by a nucleic acid of SEQ ID NO:5 or 7. Preferably, a mouse SLC26A1 polypeptide comprises: (a) a polypeptide of SEQ ID NO:10; or (b) a polypeptide encoded by a nucleic acid of SEQ ID NO:9.

Also provided are systems for recombinant expression of a SLC26 polypeptide. The system comprises: (a) a SLC26 polypeptide of the invention (representative embodiments set forth as SEQ ID NOs:2, 6, 8, and 10); and (b) a host cell expressing the SLC26 polypeptide. A host cell can comprise any suitable cell. A preferred host cell comprises a mammalian cell, more preferably a human cell.

Using the disclosed system for recombinant expression of a SLC26 polypeptide, the present invention further provides a method for identifying modulators of anion transport. Also provided are modulators of anion transport that are identified by the disclosed methods.

In a preferred embodiment of the invention a method for identifying a modulator of anion transport comprises: (a) providing a recombinant expression system whereby a functional SLC26 polypeptide is expressed in a host cell, and wherein the SLC26 polypeptide comprises a human SLC26A6a polypeptide, a mouse SLC26A6 polypeptide, or a mouse SLC26A1 polypeptide; (b) providing a test substance to the system of (a); (c) assaying a level or quality of SLC26 function in the presence of the test substance; (d) comparing the level or quality of SLC26 function in the presence of the test substance with a control level or quality of SLC26 function; and (e) identifying a test substance as an anion transport modulator by determining a level or quality of SLC26 function in the presence of the test substance as significantly changed when compared to a control level or quality of SLC26 function.

In another embodiment of the invention, a method for identifying a modulator of anion transport comprises: (a) exposing a SLC26A polypeptide to one or more test substances, wherein the SLC26A polypeptide comprises a human SLC26A6a polypeptide, a mouse SLC26A6 polypeptide, or a mouse SLC26A1 polypeptide; (b) assaying binding of a test substance to the isolated SLC26A6 polypeptide; and (c) selecting a candidate substance that demonstrates specific binding to the SLC26A6 polypeptide.

The present invention further provides methods for modulating anion transport activity in a subject. Preferably, the subject is a mammalian subject, and more preferably a human subject. Also preferably, the anion transport activity that is altered in a subject comprises an activity of a SLC26A6 polypeptide.

In one embodiment of the present invention, a method for modulating anion transport activity in a subject comprises: (a) preparing a composition comprising a SLC26 modulator identified according to the disclosed methods, and a pharmaceutically acceptable carrier; (b) administering an effective dose of the composition to a subject, whereby anion transport activity in the subject is altered.

The present invention further provides a method for activating a SLC26A1 polypeptide in a subject via administering a SLC26A1 modulator to the subject, wherein the SLC26A1 modulator comprises an impermeant anion such as Cl⁻ or formate.

Also provided is a method for modulating a SLC26A2 polypeptide or a SLC26A6 polypeptide in a subject by administering a pH modifier.

Accordingly, it is an object of the present invention to provide novel SLC26 nucleic acids and polypeptides, heterologous expression systems whereby a SLC26 polypeptide is expressed, methods and assays employing a heterologous SLC26 expression system, and methods for modulating and detecting a SLC26 polypeptide. This object is achieved in whole or in part by the present invention.

An object of the invention having been stated above, other objects and advantages of the present invention will become apparent to those skilled in the art after a study of the following description of the invention, Figures, and non-limiting Examples.

### BRIEF DESCRIPTION OF THE DRAWINGS

FIG. 1 is an alignment of a conserved SLC26 domain encompassing the Prosite “sulfate transport” signature sequence (Bucher & Bairoch, 1994; Hofmann et al., 1999) (http://www.expasy.ch/prosite/) in mouse SLC26A1 (SEQ ID NO:70), mouse SLC26A2 (SEQ ID NO:71), mouse SLC26A3 (SEQ ID NO:72), mouse SLC26A4 (SEQ ID NO:73), mouse SLC26A5 (SEQ ID NO:74), mouse SLC26A6 (SEQ ID NO:75), mouse SLC26A7 (SEQ ID NO:76), mouse SLC26A8 (SEQ ID NO:77), mouse SLC26A9 (SEQ ID NO:78), and mouse SLC26A11 (SEQ ID NO:79). The 22-residue Prosite motif is underlined in sequences that conform to the consensus (SLC26A1, SLC26A2, SLC26A3, and SLC26A11). Shading, similar residues, conservative substitutions, and weakly similar residues; asterisks (*), invariant residues.

FIG. 2 is an alignment of the first 100 amino acids of mouse and human SLC26A6a proteins (SEQ ID NOs:6 and 2, respectively). Boxed sequences, unique amino-terminal extensions predicted in the longer SLC26A6a proteins (when compared to SLC26A6b proteins); asterisk (*), predicted PKC phosphorylation sites.

FIG. 3 is the predicted sequence of mouse SLC26A1 protein (SEQ ID NO:10). (●) N-glycosylation sites; asterisk (*), PKC sites; (♦) PKA sites; (▪) combined PKC/PKA sites; (▴) tyrosine kinase sites; underline, potential transmembrane domains.

FIG. 4 presents the sequence of the proximal promoter of the mouse SLC26A6 gene (SEQ ID NO:13). Coding sequence from the 3′ end of exon 1a is underlined. A predicted CpG island includes the sequence between brackets ([ ]). Potential binding sites for transcription factors are boxed and labeled. The binding sites were predicted using the TESS (Schug & Overton, 1997); available at http://www.cbil.upenn.edu/tess) and Matinspector (Quandt et al., 1995); available from Genomatix Software GmbH of Munich Germany) programs. C/EBPbeta, CCAAT/enhancer-binding protein beta isoform; E12/E47, E2A immunoglobulin enhancer binding factors; Sp1, Sp1 transcription factors; AP-2, enhancer binding protein AP-2; GATA-1, GATA-1 transcription factor; GR, glucocorticoid receptor; PR, progesterone receptor; AP-4, enhancer binding factor AP-4; CP-2, chromosomal protein 2; NF-KB, NF kappa B transcription factor; AP-1, enhancer binding protein AP-1; MAF, c-maf transcription factor.

FIGS. 5A-5D depict the expression of human and mouse SLC26A6.

FIG. 5A is a Northern blot prepared using the human tissues indicated. The blot was hybridized with a probe designed according to sequence at the 3′ end of the SLC26A6 cDNA. Numbers at left indicate transcript size in kD.

FIG. 5B is a Northern blot prepared using human pancreatic (Panc-1) and pulmonary (Calu-3) cell lines. The blot was hybridized with a probe designed according to sequence at the 3′ end of the SLC26A6 cDNA. Numbers at left indicate transcript size in kD.

FIG. 5C is a Northern blot prepared using the mouse tissues indicated. The blot was hybridized with a probe designed according to sequence at the 3′ end of the SLC26A6 cDNA. Numbers at left indicate transcript size in kD.

FIG. 5D is a picture of a 6% acrylamide gel showing resolution of SLC26A6 RT-PCR products. RT-PCR amplification of mouse SLC26A6 was performed using a sense primer in exon 1a and an anti-sense primer in exon 4. The reactions included a water template (H₂O), intestine RNA, heart RNA, and lung RNA. An additional control reaction was performed in which the reverse transcription step was omitted (RT(−)). The SLC26A6a transcript yields a 300 base pair product due to alternative splicing of the 5′ exon 1b. The SLC26A6b transcript yields a 438 base pair product by retention of exon 1b. Both transcripts are detected in intestine, heart, and lung, but not in water and no reverse transcription controls. Numbers at left indicate amplification product size in base pairs (bp).

FIGS. 6A and 6B depict anion transport activity of mouse SLC26A6b.

FIG. 6A is a bar graph that presents DIDS (1 mM)-sensitive ³⁵SO₄²⁻ uptake (pmoVoocyte/h) in oocytes expressing SLC26A6 or SLC26A1. Control oocytes (H₂O) expressed neither SLC26A6 nor SLC26A1. Open bars, extracellular pH 7.4; Solid bars, extracellular pH 6.0; asterisk (*), statistically significant difference (p<0.01) when compared to water-injected oocytes; h, hour.

FIG. 6B is a bar graph that presents ³⁶Cl⁻ uptake (pmol/oocyte/h) in oocytes expressing SLC26A6 or SLC26A1. Control cells (H₂O) expressed neither SLC26A6 nor SLC26A 1. A second group of oocytes expressing SLC26A 1 were incubated in 25 mM SO₄²⁻ during the uptake in an attempt to stimulate Cl⁻ exchange (SLC26A1, SO₄). Open bars, extracellular pH 7.4; Solid bars, extracellular pH 6.0; h, hour.

FIG. 6C is a bar graph depicting a differential effect of extracellular Cl⁻ on ³⁵SO₄²⁻ uptake. Oocytes expressing SLC26A1, oocytes expression SLC26A6, or control oocytes (H₂O) were incubated in medium containing ³⁵SO₄²⁻ for one hour. Open bars, Cl⁻-free medium; solid gray bars, 25 mM Cl⁻ added to the medium.

FIG. 6D is a bar graph depicting an effect of pH on ³⁵SO₄²⁻ uptake. Oocytes expressing SLC26A 1, oocytes expression SLC26A6, or control oocytes (H₂O) were incubated in medium containing ³⁵SO₄²⁻ and 25 mM Cl⁻. Open bars, pH 7.4; solid bars, pH 6.0.

FIGS. 7A-7C are bar graphs that summarize cis-inhibition of Cl⁻ and SO₄²⁻ transport mediated by SLC26A6.

FIG. 7A is a bar graph that depicts cis-inhibition of Cl⁻—Cl⁻ exchange in oocytes expressing SLC26A6 or in control oocytes (H₂O). Oocytes were incubated in medium containing ³⁶Cl⁻ for one hour in the absence (control) or presence of 25 mM of the indicated anions.

FIG. 7B is a bar graph that depicts cis-inhibition of SO₄²⁻ exchange. Oocytes were incubated in medium containing ³⁵SO₄²⁻ for one hour in the absence (control) or presence of 25 mM of the indicated anions.

FIG. 7C is a bar graph that depicts trans-stimulation of SO₄²⁻ exchange. Oocytes were incubated in medium containing ³⁵SO₄²⁻ for one hour, washed three times with cold uptake medium, and then incubated for 30 minutes in the absence (control) or presence of 10 mM of the indicated anions to stimulate ³⁵SO₄²⁻ efflux.

FIGS. 8A and 8B depict oxalate and formate transport mediated by SLC26A1 and SLC26A6.

FIG. 8A is a bar graph showing oxalate uptake (pmol/oocyte/h) in oocytes expressing SLC26A1 or SLC26A6. Control oocytes (H₂O) expressed neither SLC26A6 nor SLC26A1. Asterisk (*), statistically significant difference (p<0.01) when compared to water-injected oocytes; h, hour.

FIG. 8B is a bar graph showing oxalate uptake (pmol/oocyte/h) in oocytes expressing SLC26A1 or SLC26A6. Control oocytes (H₂O) expressed neither SLC26A6 nor SLC26A1. Asterisk (*), statistically significant difference (p<0.01) when compared to water-injected oocytes; h, hour.

FIGS. 9A and 9B present a differential effect of extracellular anions on SO₄²⁻ and oxalate uptake by SLC26A1 and SLC26A6.

FIG. 9A is a bar graph showing ³⁵SO₄²⁻ uptake in oocytes expressing SLC26A 1 or in control oocytes (H₂O). Oocytes were incubated in medium containing ³⁵SO₄²⁻ for one hour in the absence (control) or presence of 25 mM of the indicated anions. Monovalent anions were observed to activate ³⁵SO₄²⁻ transport by SLC26A1, whereas they inhibit ³⁵SO₄²⁻ transport by SLC26A6 (FIG. 7B).

FIG. 9B is a bar graph showing oxalate uptake in oocytes expressing SLC26A1, oocytes expressing SLC25A6, or in control oocytes (H₂O). Oocytes were incubated in medium containing oxalate for one hour in the absence (control) or presence of 25 mM of the indicated anions. Monovalent anions activated oxalate transport by SLC26A1, whereas they inhibited oxalate transport by SLC26A6. Open bars, oocytes expressing SLC26A 1; solid bars, oocytes expressing SLC26A6.

FIGS. 10A-10C summarize anion transport activity of SLC26A2.

FIG. 10A is a bar graph showing ³⁵SO₄²⁻ uptake in oocytes expressing SLC26A2 or in control oocytes (H₂O). Oocytes were incubated in medium containing ³⁵SO₄²⁻ for one hour in the absence of extracellular Cl⁻. Open bars, extracellular pH 7.4; Solid bars, extracellular pH 6.0; h, hour.

FIG. 10B is a bar graph showing that sulphate uptake by SLC26A2-injected oocytes is cis-inhibited by extracellular Cl⁻. ³⁵SO₄²⁻ uptake was measured for one hour in oocytes expressing SLC26A2 or in control oocytes (H₂O). (−) chloride-free media; (+) media containing 25 mM Cl⁻.

FIG. 10C is a bar graph that presents ³⁶Cl⁻ uptake (pmol/oocyte/h) in oocytes expressing SLC26A2 or control cells (H₂O). Open bars, extracellular pH 7.4; Solid bars, extracellular pH 6.0; h, hour.

FIGS. 11A-11B present a functional characterization of SLC26A6 using ion-selective microelectrodes.

FIGS. 11A-11B and 12A-12B present the results of electrophysiological experiments described in Example 7.

FIG. 13 depicts chloride uptake in Xenopus oocytes expressing Xenopus xPDS2, human SLC26A3 (DRA), mouse Slc26a6, and human SLC26A6, with extracellular pH of 7.5 (open bars) or 6.0 (dark fill). Chloride uptake is significantly higher than that of water-injected oocytes (H₂O).

FIG. 14 depicts sulphate transport mediated by Xenopus oocytes expressing mouse Slc26a6, at varying concentrations of extracellular s042- and constant amounts of tracer ³⁵SO₄²⁻.

FIG. 15 depicts sulphate transport mediated by Xenopus oocytes expressing human SLC26A6, at varying concentrations of extracellular SO₄²⁻ and constant amounts of tracer ³⁵SO₄²⁻. Note the scale of the Y-axis; absolute transport rates are much lower than in oocytes expressing mouse Slc26a6 (FIG. 14).

FIG. 16 is a phylogenetic tree encompassing the ten murine Slc26 proteins and the five Xenopus laevis xSLC26 proteins. The three xPOS proteins are most homologous to Slc26a4 (Pendrin or PDS), whereas xSLC26A1 and xSLC26A6 are the clear orthologs of murine Slc26a1 and Slc26a6, respectively.

FIG. 17 depicts chloride transport mediated by oocytes expressing four of the Xenopus laevis xSLC26 anion exchangers; uptakes are significantly higher than that of water-injected oocytes (H₂O).

FIG. 18 depicts HCO₃⁻ transport mediated by xPDS2, characterized using ion-selective micro-electrodes. An experiment monitoring intracellular pH (pHᵢ) and membrane potential (Vₘ) of an xPDS2 oocyte is shown. The initial pH and rate of CO₂-induced acidification is equivalent to that of the water control. In the continuing presence of 5% CO₂/33 mM HCO₃⁻ (pH 7.5), Cl⁻ removal elicits a robust alkalinization that halts with Cl⁻ re-addition. Simultaneously, there is a pronounced and reversible depolarization not observed in control oocytes. Replacement of Na⁺ (choline) elicits no ΔpHᵢ and a small hyperpolarization as observed in control cells.

FIG. 19 depicts cis-inhibition of ³⁶Cl⁻ uptake by various anions in oocytes expressing human SLC26A3 (DRA). Oocytes were exposed to 10 mM concentrations of the anions noted during the uptake period; uptake medium for the control group did not contain anions other than ³⁶Cl⁻ and gluconate.

FIG. 20 depicts Western blotting of oocyte lysates containing the indicated SLC26 proteins, using a 1:300 titre of a C-terminal Slc26a6-specific antibody; only the core (˜85 kDa) and glycosylated (˜100 kDa) SLC26A6 and Slc26a6 proteins are detected.

FIG. 21 depicts Western blotting of oocyte lysates containing the indicated SLC26 proteins, using a 1:300 titre of an N-terminal Slc26a6-specific antibody; only the core (˜85 kDa) and glycosylated (˜100 kDa) SLC26A6 and Slc26a6 proteins are detected.

### BRIEF DESCRIPTION OF SEQUENCES IN THE SEQUENCE LISTING

Odd-numbered SEQ ID NOs:1-11 are nucleotide sequences described in Table 1. Even-numbered SEQ ID NOs:2-12 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

SEQ ID NO:13 is the mouse SLC26A6 promoter region.

SEQ ID NOs:14-55 are splice donor and acceptor sites of mouse SLC26A6, which are presented in Table 2.

SEQ ID NOs:56-61 are splice donor and acceptor sites of mouse SLC26A 1, which are presented in Table 3.

SEQ ID NOs:62-67 are primers.

SEQ ID NO:68 is a SLC26A6 conserved domain.

SEQ ID NO:69 is a SLC26 conserved domain.

SEQ ID NOs:70-79 are the SLC26 sequences indicated in Table 1, each sequence encompassing the Prosite “sulphate transport” signature sequence (Bucher & Bairoch, 1994; Hofmann et al., 1999) (http://www.expasy.ch/prosite/).

SEQ ID NOs:80-85 are the nucleic acid and amino acid sequences indicated in Table 1 of three apparent orthologs of SLC26A4 (PDS1-3) isolated from Xenopus laevis.

SEQ ID NOs:86-87 are the nucleic acid and amino acid sequences, respectively, indicated in Table 1 of SLC26A1 isolated from Xenopus laevis.

SEQ ID NOs:88-89 are the nucleic acid and amino acid sequences, respectively, indicated in Table 1 of SLC26A6 isolated from Xenopus laevis.

SEQ ID NOs:90-91 are the nucleic acid and amino acid sequences, respectively, indicated in Table 1 of SLC26A6a isolated from pig (Sus scrofa).

SEQ ID NO:92 is an amino acid sequence corresponding to residues 40-56 of the mouse SLC26A6a protein.

SEQ ID NO:93 is an amino acid sequence corresponding to residues 631-649 of the mouse SLC26A6a protein.

SEQ ID NO:94 is an amino acid sequence corresponding to residues 564-580 of the mouse SLC26A1 protein.

SEQ ID NO:95 is an amino acid sequence corresponding to residues 6-22 of the human SLC26A2 protein.

SEQ ID NO:96 is an amino acid sequence for a human SLC26A2 polypeptide.

TABLE 1
Sequence Listing Summary

| SEQ ID NO. | Description |
|---|---|
| 1-2 | human SLC26A6a |
| 3-4 | human SLC26A6b |
| 5-6 | mouse SLC26A6a |
| 7-8 | mouse SLC26A6b |
| 9-10 | mouse SLC26A1 |
| 11-12 | mouse SLC26A2 |
| 13 | mouse SLC26A6 promoter region |
| 14-55 | mouse SLC26A6 splice sites |
| 56-61 | mouse SLC26A1 splice sites |
| 62-67 | primers |
| 68 | SLC26A6 conserved domain |
| 69 | SLC26 conserved domain |
| 70 | mouse SLC26A1 sulphate transport motif |
| 71 | mouse SLC26A2 sulphate transport motif |
| 72 | mouse SLC26A3 sulphate transport motif |
| 73 | mouse SLC26A4 sulphate transport motif |
| 74 | mouse SLC26A5 sulphate transport motif |
| 75 | mouse SLC26A6 sulphate transport motif |
| 76 | mouse SLC26A7 sulphate transport motif |
| 77 | mouse SLC26A8 sulphate transport motif |
| 78 | mouse SLC26A9 sulphate transport motif |
| 79 | mouse SLC26A11 sulphate transport motif |
| 80-81 | Xenopus PDS1 |
| 82-83 | Xenopus PDS2 |
| 84-85 | Xenopus PDS3 |
| 86-87 | Xenopus SLC26A1 |
| 88-89 | Xenopus SLC26A6 |
| 90-91 | pig SLC26A6a |
| 92 | residues 40-56 of the mouse SLC26A6a protein |
| 93 | residues 631-649 of the mouse SLC26A6a protein |
| 94 | residues 564-580 of the mouse SLC26A1 protein |
| 95 | residues 6-22 of the human SLC26A2 protein |
| 96 | human SLC26A2 |

### DETAILED DESCRIPTION OF THE INVENTION

I. Definitions

While the following terms are believed to be well understood by one of ordinary skill in the art, the following definitions are set forth to facilitate explanation of the invention.

The terms “a,” and “an,” and “the” are used in accordance with long-standing convention to refer to one or more.

The term “about”, as used herein when referring to a measurable value such as a percentage of sequence identity (e.g., when comparing nucleotide and amino acid sequences as described herein below), a nucleotide or protein length, an uptake amount, a pH value, etc. is meant to encompass variations of ±20% or ±10%, more preferably ±5%, even more preferably ±1%, and still more preferably ±0.1% from the specified amount, as such variations are appropriate to perform a disclosed method or otherwise carry out the present invention.

II. SLC26 Nucleic Acids and Polypeptides

The present invention provides novel SLC26 nucleic acids and novel SLC26 polypeptides, including functional SLC26 polypeptides. The term “SLC26A” and terms including “SLC26” (e.g., SLC26A6, SLC26A1, and SLC26A2) refer generally to isolated SLC26 nucleic acids, isolated polypeptides encoded by SLC26 nucleic acids, and activities thereof. SLC26 nucleic acids and polypeptides can be derived from any organism.

The term “isolated”, as used in the context of a nucleic acid or polypeptide, indicates that the nucleic acid or polypeptide exists apart from its native environment and is not a product of nature. An isolated nucleic acid or polypeptide can exist in a purified form or can exist in a non-native environment such as a transgenic host cell.

The terms “SLC26” and terms including “SLC26” also refer to polypeptides comprising Na⁺-independent anion transporters that transport SO₄²⁻, Cl⁻, formate, and/or oxalate, and to nucleic acids encoding the same.

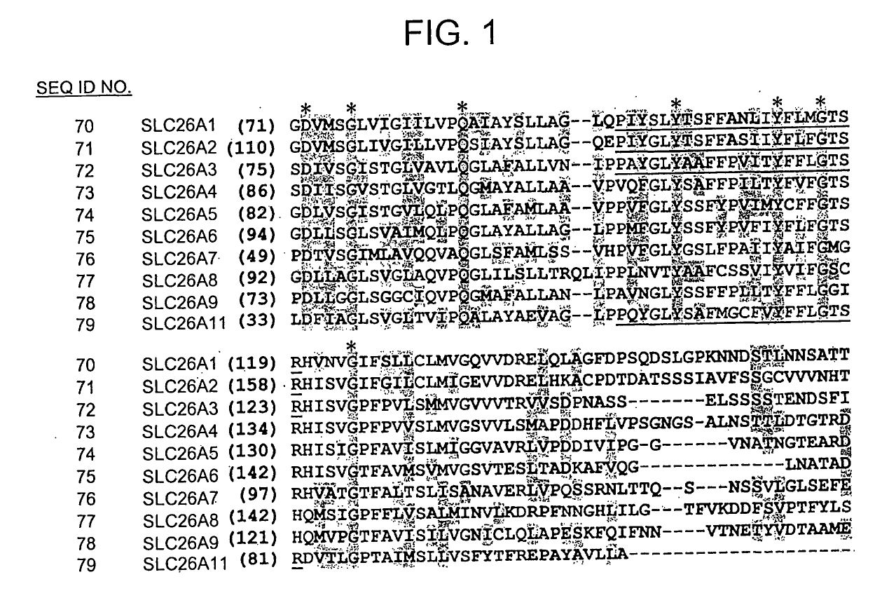

A region within the central hydrophobic core of SLC26 polypeptides includes a 22-residue “sulphate transport” consensus signature, Prosite motif PS01130 (Bucher & Bairoch, 1994; Hofmann et al., 1999) (http://www.expasy.ch/prosite/), which was initially defined by comparison of the first mammalian family members with homologues in lower organisms. An alignment of this region is presented in FIG. 1. SLC26A6 functions as a sulphate transporter (Example 6), despite its lack of a consensus “sulphate transport” sequence, and thus the functional significance of this sequence motif is unclear. Within this region, many of the SLC26 proteins also share the sequence -GTSRHISV- (SEQ ID NO:69), whereas mouse SLC26A8 and mouse SLC26A9 depart from this consensus. The Prosite sulphate transport region also contains a total of seven invariant residues, which are likely play a role in anion transport (FIG. 1).

There is a second cluster of invariant residues at the C-terminal end of the hydrophobic core, in a conserved area defined by Saier et al (Saier et al., 1999). This region includes the triplet -NQE-, residues 417-419 of mouse SLC26A6, which is conservatively variable only in SLC26A8 (-NQD-). Three invariant residues in this section, E419, N425, and L483 in mouse SLC26A2, have been shown to have functional significance in SHST1, a SLC26 homologue from the plant S. hamata (Khurana et al., 2000). Moreover, two of these invariant residues are mutated (N425D and L483P) in patients with a severe defect in human SLC26A2, causing achondrogenesis type 1B and/or atelosteogenesis type 2. The SLC26A2 N425D mutant has further been shown to be non-functional in Xenopus oocytes (Karniski, 1989).

The C-terminal cytoplasmic domain of SLC26 proteins encompasses the STAS (Sulphate Transporter and Anti-Sigma) domain, recently defined by the homology between the SLC26 proteins and bacterial anti-sigma factor antagonists (Aravind & Koonin, 2000). Structural features of this domain have been predicted from the NMR analysis of the anti-sigma factor SPOIIAA (Aravind & Koonin, 2000), and include a characteristic α-helical handle. There is also a highly conserved loop interspersed between a β-pleated sheet and α-helix, just upstream of the α-helical handle. This loop and β-pleated sheet have been proposed to play a role in nucleotide binding and hydrolysis, in analogy to the known biochemistry of the anti-sigma factor antagonists (Aravind & Koonin, 2000). The loop is highly conserved in SLC26 proteins and contains two invariant residues, D660 and L667 of mouse SLC26A2.

The STAS domain also contains a highly variable loop just proximal to the β-pleated sheet and putative nucleotide binding loop (Aravind & Koonin, 2000). This variable loop is the site of significant insertions in SLC26 proteins. The largest known insertion comprises 150 amino acids in the case of human SLC26A8. Interestingly, no such insertion is present in bacterial SLC26 homologues, and this loop is the shortest in SLC26A11, which is arguably the most primeval of the mammalian SLC26 paralogs.

The present invention provides novel human SLC26A6a polypeptides, which is the shorter of two isoforms encoded by SLC26A6 and contains a unique amino-terminal extension. SLC26A6b is the longer isoform and lacks the amino-terminal extension. Also provided are novel nucleic acids encoding a human SLC26A6a polypeptide. A representative SLC26A6a nucleic acid of the present invention is set forth as SEQ ID NO:1, which encodes a SLC26A6a polypeptide set forth as SEQ ID NO:2.

The present invention further provides novel mouse SLC26A6 polypeptides, including SLC26A6a and SLC26A6b isoforms, and nucleic acids encoding the same. Representative mouse SLC26A6a and mouse SLC26A6b nucleic acids are set forth as SEQ ID NOs:3 and 5, respectively. Representative mouse SLC26A6a and mouse SLC26A6b polypeptides are set forth as SEQ ID NOs:4 and 6, respectively.

Also provided are novel mouse SLC26A1 polypeptides and nucleic acids encoding the same. A representative SLC26A1 nucleic acid is set forth as SEQ ID NO:9, which encodes a SLC26A1 polypeptide set forth as SEQ ID NO:10.

Representative polypeptides and nucleic acids that are also provided comprise orthologs from porcine and Xenopus sources, as disclosed in the Examples and in SEQ ID NOs: 80-91, and the methods, definitions, sequence comparison, and hybridization conditions set forth herein are equally applicable to the orthologs.

As disclosed further herein below, the present invention also provides a system for functional expression of a SLC26 polypeptide, including but not limited to a SLC26A6 polypeptide, a SLC26A1 polypeptide, and a SLC26A2 polypeptide. The system employs a recombinant SLC26 nucleic acid, including any one of odd-numbered SEQ ID NOs:1-11.

II.A. SLC26 Nucleic Acids

The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” or “nucleic acid” can also be used in place of “gene,” “cDNA,” “mRNA,” or “cRNA.” Nucleic acids can be synthesized, or can be derived from any biological source, including any organism. Representative methods for cloning a full-length SLC26 cDNA are described in Example 1.

The terms “SLC26” and terms including “SLC26” (e.g., SLC26A1, SLC26A2, and SLC26A6) are used herein to refer to nucleic acids that encode a SLC26 polypeptide. Thus, the term “SLC26” refers to isolated nucleic acids of the present invention comprising: (a) a nucleotide sequence comprising the nucleotide sequence of any one of odd-numbered SEQ ID NOs: 1-11; or (b) a nucleotide sequence substantially identical to any one of odd-numbered SEQ ID NOs:1-11.

The term “substantially identical”, as used herein to describe a degree of similarity between nucleotide sequences, refers to two or more sequences that have at least about least 60%, preferably at least about 70%, more preferably at least about 80%, more preferably about 90% to about 99%, still more preferably about 95% to about 99%, and most preferably about 99% nucleotide identity, when compared and aligned for maximum correspondence, as measured using one of the following sequence comparison algorithms or by visual inspection. Preferably, the substantial identity exists in nucleotide sequences of at least about 100 residues, more preferably in nucleotide sequences of at least about 150 residues, and most preferably in nucleotide sequences comprising a full length coding sequence. The term “full length” is used herein to refer to a complete open reading frame encoding a functional SLC26 polypeptide, as described further herein below. Methods for determining percent identity between two polypeptides are defined herein below under the heading “Nucleotide and Amino Acid Sequence Comparisons”.

In one aspect, substantially identical sequences can be polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise one or more residue changes, a deletion of residues, or an insertion of additional residues.

Another indication that two nucleotide sequences are substantially identical is that the two molecules hybridize specifically to or hybridize substantially to each other under stringent conditions. In the context of nucleic acid hybridization, two nucleic acid sequences being compared can be designated a “probe” and a “target.” A “probe” is a reference nucleic acid molecule, and a “target” is a test nucleic acid molecule, often found within a heterogeneous population of nucleic acid molecules. A “target sequence” is synonymous with a “test sequence.”

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of odd-numbered SEQ ID NOs:1-11. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further under the heading “SLC26 Polypeptides” herein below. Nucleic acid molecules that do not hybridize to each other under stringent conditions are still substantially identical if the corresponding proteins are substantially identical. This can occur, for example, when two nucleotide sequences comprise conservatively substituted variants as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98.

The term “SLC26” also encompasses nucleic acids comprising subsequences and elongated sequences of a SLC26 nucleic acid, including nucleic acids complementary to a SLC26 nucleic acid, SLC26 RNA molecules, and nucleic acids complementary to SLC26 RNAs (cRNAs).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences,” as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The present invention also provides chimeric genes comprising the disclosed SLC26 nucleic acids and recombinant SLC26 nucleic acids. Thus, also included are constructs and vectors comprising SLC26 nucleic acids.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “chimeric gene,” as used herein, refers to a promoter region operatively linked to a SLC26 sequence, including a SLC26 cDNA, a SLC26 nucleic acid encoding an antisense RNA molecule, a SLC26 nucleic acid encoding an RNA molecule having tertiary structure (e.g., a hairpin structure) or a SLC26 nucleic acid encoding a double-stranded RNA molecule.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, which enable its replication in a host cell.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a SLC26 polypeptide, as described further herein below.

The term “construct”, as used herein to describe a type of construct comprising an expression construct, refers to a vector further comprising a nucleotide sequence operatively inserted with the vector, such that the nucleotide sequence is recombinantly expressed.

The terms “recombinantly expressed” or “recombinantly produced” are used interchangeably to refer generally to the process by which a polypeptide encoded by a recombinant nucleic acid is produced.

Thus, preferably, recombinant SLC26 nucleic acids comprise heterologous nucleic acids. The term “heterologous nucleic acids” refers to a sequence that originates from a source foreign to an intended host cell or, if from the same source, is modified from its original form. A heterologous nucleic acid in a host cell can comprise a nucleic acid that is endogenous to the particular host cell but has been modified, for example by mutagenesis or by isolation from native cis-regulatory sequences. A heterologous nucleic acid also includes non-naturally occurring multiple copies of a native nucleotide sequence. A heterologous nucleic acid can also comprise a nucleic acid that is incorporated into a host cell's nucleic acids at a position wherein such nucleic acids are not ordinarily found.

Nucleic acids of the present invention can be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) (1989) Molecular Cloninq: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/N.Y.; Ausubel (ed.) (1995) Short Protocols in Molecular Biology, 3rd ed. Wiley, New York.

II.B. SLC26 Polypeptides

The present invention provides novel SLC26 polypeptides comprising one of a human SLC26A6a polypeptide, a mouse SLC26A6a polypeptide, a mouse SLC26A6b polypeptide, and a mouse SLC26A1 polypeptide. Representative embodiments are set forth as even-numbered SEQ ID NOs:2, 6, 8, and 10, respectively. Preferably, an isolated SLC26 polypeptide of the present invention comprises a recombinantly expressed SLC26 polypeptide. Also preferably, isolated SLC26 polypeptides comprise functional SLC26 polypeptides.

Thus, novel SLC26 polypeptides useful in the methods of the present invention comprise: (a) a polypeptide encoded by a nucleic acid of any one of odd-numbered SEQ ID NOs:1-11; (b) a polypeptide encoded by a nucleic acid substantially identical to any one of odd-numbered SEQ ID NOs:1-11; (c) a polypeptide comprising an amino acid sequence of any one of even-numbered SEQ ID NOs:2-12; or (d) a polypeptide substantially identical to any one of even-numbered SEQ ID NOs:2-12.

Representative polypeptides, and nucleic acids encoding the same, that are provided comprise orthologs from porcine and Xenopus sources, as disclosed in the Examples and in SEQ ID NOs: 80-91, and the definitions, sequence comparison, and hybridization conditions set forth herein are equally applicable to the orthologs.

The term “substantially identical”, as used herein to describe a level of similarity between SLC26 and a protein substantially identical to a SLC26 protein, refers to a sequence that is at least about 35% identical to any of even-numbered SEQ ID NOs:2-12, when compared over the full length of a SLC26 protein. Preferably, a protein substantially identical to a SLC26 protein comprises an amino acid sequence that is at least about 35% to about 45% identical to any one of even-numbered SEQ ID NOs:2-12, more preferably at least about 45% to about 55% identical to any one of even-numbered SEQ ID NOs:2-12, even more preferably at least about 55% to about 65% identical to any one of even-numbered SEQ ID NOs:2-12, still more preferably preferably at least about 65% to about 75% identical to any one of even-numbered SEQ ID NOs:2-12, still more preferably preferably at least about 75% to about 85% identical to any one of even-numbered SEQ ID NOs:2-12, still more preferably preferably at least about 85% to about 95% identical to any one of even-numbered SEQ ID NOs:2-12, and still more preferably at least about 95% to about 99% identical to any one of even-numbered SEQ ID NOs:2-12 when compared over the full length of a SLC26 polypeptide. The term “full length” refers to a functional SLC26 polypeptide, as described further herein below. Methods for determining percent identity between two polypeptides are also defined herein below under the heading “Nucleotide and Amino Acid Sequence Comparisons”.

The term “substantially identical,” when used to describe polypeptides, also encompasses two or more polypeptides sharing a conserved three-dimensional structure. Computational methods can be used to compare structural representations, and structural models can be generated and easily tuned to identify similarities around important active sites or ligand binding sites. See Saqi et al. (1999) Bioinformatics 15:521-522; Barton (1998) Acta Crystallogr D Biol Crystallogr 54:1139-1146; Henikoff et al. (2000) Electrophoresis 21:1700-1706; and Huang et al. (2000) Pac Symp Biocomput:230-241.

Substantially identical proteins also include proteins comprising amino acids that are functionally equivalent to amino acids of any one of even-numbered SEQ ID NOs:2-12. The term “functionally equivalent” in the context of amino acids is known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The term “substantially identical” also encompasses polypeptides that are biologically functional equivalents of a SLC26 polypeptide. The term “functional” includes an activity of an SLC26 polypeptide in transporting anions across a membrane. Preferably, such transport shows a magnitude and anion selectivity that is substantially similar to that of a cognate SLC26 polypeptide in vivo. Preferably, the term “functional” also refers to similar kinetics of activation and inactivation of anion transport activity. Representative methods for assessing anion transport activity are described herein below.

The present invention also provides functional fragments of a SLC26 polypeptide. Such functional portion need not comprise all or substantially all of the amino acid sequence of a native SLC26 gene product.

The present invention also includes functional polypeptide sequences that are longer sequences than that of a native SLC26 polypeptide. For example, one or more amino acids can be added to the N-terminus or C-terminus of a SLC26 polypeptide. Such additional amino acids can be employed in a variety of applications, including but not limited to purification applications. Methods of preparing elongated proteins are known in the art.

II.C. Nucleotide and Amino Acid Sequence Comparisons

The terms “identical” or “percent identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological function of a SLC26 nucleic acid or a SLC26 polypeptide.

For comparison of two or more sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example, by the local homology algorithm of Smith & Waterman (1981) Adv Appl Math 2:482-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.) (1995) Short Protocols in Molecular Biology, 3rd ed. Wiley, New York.

A preferred algorithm for determining percent sequence identity and sequence similarity is the BLAST algorithm, which is described in Altschul et al. (1990) J Mol Biol 215:403-410. Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) Proc Natl Acad Sci USA 89:10915-10919.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences that would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

III. Methods for Detecting a SLC26 Nucleic Acid

In another aspect of the invention, a method is provided for detecting a nucleic acid molecule that encodes a SLC26 polypeptide. Such methods can be used to detect SLC26 gene variants or altered gene expression. For example, detection of a change in SLC26 sequence or expression can be used for diagnosis of SLC26-related diseases, disorders, and drug interactions. Preferably, the nucleic acids used for this method comprise sequences set forth as any one of SEQ ID NOs:1, 5, 7, and 9.

Sequences detected by methods of the invention can detected, subcloned, sequenced, and further evaluated by any measure well known in the art using any method usually applied to the detection of a specific DNA sequence. Thus, the nucleic acids of the present invention can be used to clone genes and genomic DNA comprising the disclosed sequences. Alternatively, the nucleic acids of the present invention can be used to clone genes and genomic DNA of related sequences. Using the nucleic acid sequences disclosed herein, such methods are known to one skilled in the art. See e.g., Sambrook et al., eds (1989) Molecular Cloning, Cold Spring Harbor Laboratory Press, Cold Spring Harbor, N.Y. Representative methods are also disclosed in Examples 1-4.

In one embodiment of the invention, levels of a SLC26 nucleic acid molecule are measured by, for example, using an RT-PCR assay. See Chiang (1998) J Chromatogr A 806:209-218, and references cited therein.

In another embodiment of the invention, genetic assays based on nucleic acid molecules of the present invention can be used to screen for genetic variants, for example by allele-specific oligonucleotide (ASO) probe analysis (Conner et al., 1983), oligonucleotide ligation assays (OLAs) (Nickerson et al., 1990), single-strand conformation polymorphism (SSCP) analysis (Orita et al., 1989), SSCP/heteroduplex analysis, enzyme mismatch cleavage, direct sequence analysis of amplified exons (Kestila et al., 1998; Yuan et al., 1999), allele-specific hybridization (Stoneking et al., 1991), and restriction analysis of amplified genomic DNA containing the specific mutation. Automated methods can also be applied to large-scale characterization of single nucleotide polymorphisms (Wang et al., 1998; Brookes, 1999). Preferred detection methods are non-electrophoretic, including, for example, the TAQMAN™ allelic discrimination assay, PCR-OLA, molecular beacons, padlock probes, and well fluorescence. See Landegren et al. (1998) Genome Res 8:769-776 and references cited therein.

IV. System for Recombinant Expression of a SLC26 Polypeptide

The present invention further provides a system for expression of a recombinant SLC26 polypeptide of the present invention. Such a system can be used for subsequent purification and/or characterization of a SLC26 polypeptide. For example, a purified SLC26A6 polypeptide can be used as an immunogen for the production of an SLC26 antibody, described further herein below.

A system for recombinant expression of a SLC26 polypeptide can be used for the identification of modulators of anion transport. In one embodiment of the invention, a method is provided for identification of SLC26 modulators, as described herein below. Alternatively, the disclosed SLC26 polypeptides can be used as a control anion transporter when testing any other molecule for anion transport activity. For example, the present invention discloses that SLC26A6 is a chloride transporter, and thus a system for recombinant SLC26A6 expression can be used as a positive control in an assay to determine chloride transport of a test polypeptide. Such test polypeptides can include candidates for any one of a variety of hereditary and acquired disease such as cystic fibrosis, nephrolithiasis, and cholera.

The term “expression system” refers to a host cell comprising a heterologous nucleic acid and the polypeptide encoded by the heterologous nucleic acid. For example, a heterologous expression system can comprise a host cell transfected with a construct comprising a recombinant SLC26 nucleic acid, a host cell transfected with SLC26 cRNA, or a cell line produced by introduction of heterologous nucleic acids into a host cell genome.

A system for recombinant expression of a SLC26 polypeptide can comprise: (a) a recombinantly expressed SLC26 polypeptide; and (b) a host cell comprising the recombinantly expressed SLC26 polypeptide. For example, a SLC26 cRNA can be transcribed in vitro and then introduced into a host cell, whereby a SLC26 polypeptide is expressed. In a preferred embodiment of the invention, SLC26 cRNA is provided to a host cell by direct injection of a solution comprising the SLC26 cRNA, as described in Example 5. The system can further comprise a plurality of different SLC26 polypeptides.

A system for recombinant expression of a SLC26 polypeptide can also comprise: (a) a construct comprising a vector and a nucleic acid molecule encoding a SLC26 polypeptide operatively linked to a heterologous promoter; and (b) a host cell comprising the construct of (a), whereby the host cell expresses a SLC26 polypeptide. The system can further comprise constructs encoding a plurality of different SLC26 polypeptides. Additionally, a single construct itself can encode a plurality of different SLC26 polypeptides.

Isolated polypeptides and recombinantly produced polypeptides can be purified and characterized using a variety of standard techniques that are known to the skilled artisan. See e.g., Schröder & Lübke (1965) The Peptides. Academic Press, New York; Schneider & Eberle (1993) Peptides, 1992: Proceedings of the Twenty-Second European Peptide Symposium, September 13-19, 1992, Interlaken. Switzerland. Escom, Leiden; Bodanszky (1993) Principles of Peptide Synthesis, 2nd rev. ed. Springer-Verlag, Berlin; N.Y.; Ausubel (ed.) (1995) Short Protocols in Molecular Biology, 3rd ed. Wiley, New York.

Preferably, a recombinantly expressed SLC26 polypeptide comprises a functional anion transporter. Thus, a recombinantly expressed SLC26 polypeptide preferably displays transport of Cl⁻, SO₄²⁻, oxalate, and/or formate across a lipid bilayer or membrane. Also preferably, a recombinant SLC26 polypeptide shows ion selectivity similar to a native SLC26 polypeptide. Representative methods for determining SLC26 function are described herein below.

IV.A. Expression Constructs

A construct for expression of a SLC26 polypeptide includes a vector and a SLC26 nucleotide sequence, wherein the SLC26 nucleotide sequence is operatively linked to a promoter sequence. A construct for recombinant SLC26 expression can also comprise transcription termination signals and sequences required for proper translation of the nucleotide sequence. Preparation of an expression construct, including addition of translation and termination signal sequences, is known to one skilled in the art.

Recombinant production of a SLC26 polypeptide can be directed using a constitutive promoter or an inducible promoter. Representative promoters that can be used in accordance with the present invention include Simian virus 40 early promoter, a long terminal repeat promoter from retrovirus, an actin promoter, a heat shock promoter, and a metallothien protein.

Suitable vectors that can be used to express a SLC26 polypeptide include but are not limited to viruses such as vaccinia virus or adenovirus, baculovirus vectors, yeast vectors, bacteriophage vectors (e.g., lambda phage), plasmid and cosmid DNA vectors, transposon-mediated transformation vectors, and derivatives thereof.

Constructs are introduced into a host cell using a transfection method compatible with the vector employed. Standard transfection methods include electroporation, DEAE-Dextran transfection, calcium phosphate precipitation, liposome-mediated transfection, transposon-mediated transformation, infection using a retrovirus, particle-mediated gene transfer, hyper-velocity gene transfer, and combinations thereof.

IV.B. Host Cells

The term “host cell”, as used herein, refers to a cell into which a heterologous nucleic acid molecule can be introduced. Any suitable host cell can be used, including but not limited to eukaryotic hosts such as mammalian cells (e.g., HeLa cells, CV-1 cells, COS cells), amphibian cells (e.g., Xenopus oocytes), insect cells (e.g., Sf9 cells), as well as prokaryotic hosts such as E. coli and Bacillus subtilis. Preferred host cells are amphibian cells such as Xenopus oocytes. Also preferably, a host cell substantially lacks a SLC26 polypeptide.

A host cell strain can be chosen which modulates the expression of the recombinant sequence, or modifies and processes the gene product in the specific fashion desired. For example, different host cells have characteristic and specific mechanisms for the translational and post-transactional processing and modification (e.g., glycosylation, phosphorylation of proteins). Appropriate cell lines or host systems can be chosen to ensure the desired modification and processing of the foreign protein expressed. For example, expression in a bacterial system can be used to produce a non-glycosylated core protein product, and expression in yeast will produce a glycosylated product.

The present invention further encompasses recombinant expression of a SLC26 polypeptide in a stable cell line. Methods for generating a stable cell line following transformation of a heterologous construct into a host cell are known in the art. See e.g., Joyner (1993) Gene Targeting: A Practical Approach. Oxford University Press, Oxford/N.Y. Thus, transformed cells, tissues, or non-human organisms are understood to encompass not only the end product of a transformation process, but also transgenic progeny or propagated forms thereof.

The present invention further encompasses cryopreservation of cells expressing a recombinant SLC26 polypeptide as disclosed herein. Thus, transiently transfected cells and cells of a stable cell line expressing SLC26 can be frozen and stored for later use. Frozen cells can be readily transported for use at a remote location.

Cryopreservation media generally consists of a base medium, cryopreservative, and a protein source. The cryopreservative and protein protect the cells from the stress of the freeze-thaw process. For serum-containing medium, a typical cryopreservation medium is prepared as complete medium containing 10% glycerol; complete medium containing 10% DMSO (dimethylsulfoxide), or 50% cell-conditioned medium with 50% fresh medium with 10% glycerol or 10% DMSO. For serum-free medium, typical cryopreservation formulations include 50% cell-conditioned serum free medium with 50% fresh serum-free medium containing 7.5% DMSO; or fresh serum-free medium containing 7.5% DMSO and 10% cell culture grade DMSO. Preferably, a cell suspension comprising about 10⁶ to about 10⁷ cells per ml is mixed with cryopreservation medium.

Cells are combined with cryopreservation medium in a vial or other container suitable for frozen storage, for example NUNC® CRYOTUBES™ (available from Applied Scientific of South San Francisco, Calif.). Cells can also be aliquotted to wells of a multi-well plate, for example a 96-well plate designed for high-throughput assays, and frozen in plated format.

Cells are preferably cooled from room temperature to a storage temperature at a rate of about −1° C. per minute. The cooling rate can be controlled, for example, by placing vials containing cells in an insulated water-filled reservoir having about 1 liter liquid capacity, and placing such cube in a −70° C. mechanical freezer. Alternatively, the rate of cell cooling can be controlled at about −1° C. per minute by submersing vials in a volume of liquid refrigerant such as an aliphatic alcohol, the volume of liquid refrigerant being more than fifteen times the total volume of cell culture to be frozen, and placing the submersed culture vials in a conventional freezer at a temperature below about −70° C. Commercial devices for freezing cells are also available, for example, the Planer Mini-Freezer R202/200R (Planer Products Ltd. of Great Britain) and the BF-5 Biological Freezer (Union Carbide Corporation of Danbury, Conn., United States of America). Preferably, frozen cells are stored at or below about −70° C. to about −80° C., and more preferably at or below about −130° C.

To obtain the best possible cell survival, thawing of the cells must be performed as quickly as possible. Once a vial or other reservoir containing frozen cells is removed from storage, it should be placed directly into a 37° C. water bath and gently shaken until it is completely thawed. If cells are particularly sensitive to cryopreservatives, the cells are centrifuged to remove cryopreservative prior to further growth.

Additional methods for preparation and handling of frozen cells can be found in Freshney (1987) Culture of Animal Cells: A Manual of Basic Technique, 2nd ed. A. R. Liss, New York and in U.S. Pat. Nos. 6,176,089; 6,140,123; 5,629,145; and 4,455,842; among other places.

V. Transgenic Animals

The present invention also provides a transgenic animal comprising a disruption of SLC26A6, SLC26A1, or SLC26A2 gene expression. Altered gene expression can include expression of an altered level or mutated variant of a SLC26A6, SLC26A 1, or SLC26A2 gene. The present invention provides nucleic acids encoding SLC26A6, SLC26A1, and SLC26A2 that can be used to prepare constructs for generating a transgenic animal. Also provided is genomic localization data useful for preparation of constructs targeted to the SLC26A6, SLC26A 1, or SLC26A2 locus.

In one embodiment of the present invention, the transgenic animal can comprise a mouse with targeted modification of the mouse SLC26A6, SLC26A1, or SLC26A2 locus and can further comprise mice strains with complete or partial functional inactivation of the SLC26A6, SLC26A1, or SLC26A2 genes in all somatic cells.

In an alternative embodiment, a transgenic animal in accordance with the present invention is prepared using anti-sense or ribozyme SLC26A6, SLC26A1, or SLC26A2 constructs, driven by a universal or tissue-specific promoter, to reduce levels of SLC26 gene expression in somatic cells, thus achieving a “knock-down” phenotype. The present invention also provides the generation of murine strains with conditional or inducible inactivation of SLC26A6, SLC26A1, SLC26A2, or a combination thereof. Such murine strains can also comprise additional synthetic or naturally occurring mutations, for example a mutation in any other SLC26 gene.

The present invention also provides mice strains with specific “knocked-in” modifications in the SLC26A6, SLC26A 1, or SLC26A2 genes, for example to create an over-expression or dominant negative phenotype. Thus, “knocked-in” modifications include the expression of both wild type and mutated forms of a nucleic acid encoding a SLC26A6, SLC26A 1, or SLC26A2 polypeptide.

Techniques for the preparation of transgenic animals are known in the art. Exemplary techniques are described in U.S. Pat. No. 5,489,742 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species), the entire contents of each of which are herein incorporated by reference.

For example, a transgenic animal of the present invention can comprises a mouse with targeted modification of the mouse SLC26A6, SLC26A 1, or SLC26A2 gene. Mice strains with complete or partial functional inactivation of the SLC26A6, SLC26A 1, or SLC26A2 genes in all somatic cells are generated using standard techniques of site-specific recombination in murine embryonic stem cells. See Capecchi, M. R. (1989) Science 244(4910):1288-92; Thomas, K. R., and Capecchi, M. R. (1990) Nature 346(6287):847-50; Delpire, E., et al. (1999) Nat Genet 22(2):192-5.

VI. SLC26 Antibodies

In another aspect of the invention, a method is provided for producing an antibody that specifically binds a SLC26 polypeptide. According to the method, a full-length recombinant SLC26 polypeptide, or fragment thereof, is formulated so that it can be used as an effective immunogen, and used to immunize an animal so as to generate an immune response in the animal. The immune response is characterized by the production of antibodies that can be collected from the blood serum of the animal. The present invention also provides antibodies produced by methods that employ the novel SLC26 polypeptides disclosed herein, including any one of SEQ ID NOs:2, 6, 8, and 10.

The term “antibody” refers to an immunoglobulin protein, or functional portion thereof, including a polyclonal antibody, a monoclonal antibody, a chimeric antibody, a hybrid antibody, a single chain antibody, a mutagenized antibody, a humanized antibody, and antibody fragments that comprise an antigen binding site (e.g., Fab and Fv antibody fragments). In a preferred embodiment of the invention, a SLC26 antibody comprises a monoclonal antibody. Thus, the present invention also encompasses antibodies and cell lines that produce monoclonal antibodies as described herein.

The term “specifically binds”, when used to describe binding of an antibody to a SLC26 polypeptide, refers to binding to a SLC26 polypeptide in a heterogeneous mixture of other polypeptides.

The phrases “substantially lack binding” or “substantially no binding”, as used herein to describe binding of an antibody to a control polypeptide or sample, refers to a level of binding that encompasses non-specific or background binding, but does not include specific binding.

Techniques for preparing and characterizing antibodies are known in the art. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054561.

SLC26 antibodies prepared as disclosed herein can be used in methods known in the art relating to the localization and activity of SLC26 polypeptides, e.g., for cloning of nucleic acids encoding a SLC26 polypeptide, immunopurification of a SLC26 polypeptide, imaging a SLC26 polypeptide in a biological sample, and measuring levels of a SLC26 polypeptide in appropriate biological samples. To perform such methods, an antibody of the present invention can further comprise a detectable label, including but not limited to a radioactive label, a fluorescent label, an epitope label, and a label that can be detected in vivo. Methods for selection of a label suitable for a particular detection technique, and methods for conjugating to or otherwise associating a detectable label with an antibody are known to one skilled in the art.

VII. SLC26 Modulators

The present invention further discloses assays to identify modulators of SLC26 activity. An assay can employ a system for expression of a SLC26 polypeptide, as disclosed herein above, or an isolated SLC26 polypeptide produced in such a system. The present invention also provides modulators of anion transport activity identified using the disclosed methods.

The term “modulate” means an increase, decrease, or other alteration of any or all chemical and biological activities or properties of a SLC26 polypeptide. Thus, the method for identifying modulators involves assaying a level or quality of SLC26 function.

A method for identifying a modulator of anion transport can comprise: (a) providing a recombinant expression system whereby a SLC26 polypeptide is expressed in a host cell, and wherein the SLC26 polypeptide comprises a human SLC26A6a polypeptide, a mouse SLC26A6 polypeptide, or a mouse SLC26A1 polypeptide; (b) providing a test substance to the system of (a); (c) assaying a level or quality of SLC26 function in the presence of the test substance; (d) comparing the level or quality of SLC26 function in the presence of the test substance with a control level or quality of SLC26 function; and (e) identifying a test substance as an anion transport modulator by determining a level or quality of SLC26 function in the presence of the test substance as significantly changed when compared to a control level or quality of SLC26 function.

In one embodiment of the invention, assaying SLC26 function comprises determining a level of SLC26 expression.

In another embodiment of the invention, assaying SLC26 function comprises assaying binding activity of a recombinantly expressed SLC26 polypeptide. For example, a SLC26 activity can comprise an amount or a strength of binding of a modulator to a SLC26 polypeptide.

In still another embodiment of the invention, assaying SLC26 function can comprise assaying an active conformation of a SLC26 polypeptide.

In a preferred embodiment of the invention, assaying SLC26 activity comprises assaying anion transport activity of a recombinantly expressed SLC26 polypeptide. A representative level of SLC26 activity can thus comprise an amount of anion transport or a peak level of anion transport, measurable as described in Example 6. A representative quality of SLC26 activity can comprise, for example, anion selectivity of a SLC26A, pH sensitivity of anion transport, and pharmacological sensitivity of a SLC26 polypeptide. The electrophysiological behavior of SLC26A6 and other SLC26 polypeptides also provides a signature for transport activity.

A control level or quality of SLC26 activity refers to a level or quality of wild type SLC26 activity. Preferably, a system for recombinant expression of a SLC26 polypeptide comprises any one of even-numbered SEQ ID NOs:2-12. When evaluating the modulating capacity of a test substance, a control level or quality of SLC26 activity comprises a level or quality of activity in the absence of a test substance.

The term “significantly changed”, as used herein to refer to an altered level or activity of a SLC26 polypeptide, refers to a quantified change in a measurable quality that is larger than the margin of error inherent in the measurement technique, preferably an increase or decrease by about 2-fold or greater relative to a control measurement, more preferably an increase or decrease by about 5-fold or greater, and most preferably an increase or decrease by about 10-fold or greater.

Modulators identified by the disclosed methods can comprise agonists and antagonists. As used herein, the term “agonist” means a substance that activates, synergizes, or potentiates the biological activity of a SLC26 polypeptide. As used herein, the term “antagonist” refers to a substance that blocks or mitigates the biological activity of a SLC26 polypeptide. A modulator can also comprise a ligand or a substance that specifically binds to a SLC26 polypeptide. Activity and binding assays for the determination of a SLC26 modulator can be performed in vitro or in vivo.

In one embodiment of the invention, such assays are useful for the identification of SLC26 modulators that can be developed for the treatment and/or diagnosis of SLC26-related disorders, as described further herein below under the heading “Therapeutic Applications.”

In another embodiment of the invention, assays using a recombinant SLC26 polypeptide can be performed for the purpose of prescreening bioactive agents, wherein an interaction between the agent and SLC26 is undesirable. Thus, drugs intended for administration to a subject for the treatment of a non-SLC26-related disorder can be tested for SLC26 modulating activity that can result in undesirable side effects. The disclosed assays and methods enable pre-screening of bioactive agents under development to identify deleterious effects of anion transport.

In still another embodiment of the invention, an assay disclosed herein can be used to characterize a mutant SLC26 polypeptide, for example a mutant polypeptide that is linked to a disorder of anion transport. Recombinant expression of mutated SLC26 polypeptides will permit further analysis of disorder-related SLC26 anion transporters.

In accordance with the present invention there is also provided a rapid and high throughput screening method that relies on the methods described herein. This screening method comprises separately contacting a SLC26 polypeptide with a plurality of test substances. In such a screening method the plurality of target substances preferably comprises more than about 10⁴ samples, or more preferably comprises more than about 10⁵ samples, and still more preferably more than about 10⁶ samples.

VII.A. Test Substances

A potential modulator assayed using the methods of the present invention comprises a candidate substance. As used herein, the terms “candidate substance” and “test substance” are used interchangeably, and each refers to a substance that is suspected to interact with a SLC26 polypeptide, including any synthetic, recombinant, or natural product or composition. A test substance suspected to interact with a polypeptide can be evaluated for such an interaction using the methods disclosed herein.

Representative test substances include but are not limited to peptides, oligomers, nucleic acids (e.g., aptamers), small molecules (e.g., chemical compounds), antibodies or fragments thereof, nucleic acid-protein fusions, any other affinity agent, and combinations thereof. A test substance can additionally comprise a carbohydrate, a vitamin or derivative thereof, a hormone, a neurotransmitter, a virus or receptor binding domain thereof, an opsin or rhodopsin, an odorant, a phermone, a toxin, a growth factor, a platelet activation factor, a neuroactive peptide, or a neurohormone. A candidate substance to be tested can be a purified molecule, a homogenous sample, or a mixture of molecules or compounds.

The term “small molecule” as used herein refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Test substances can be obtained or prepared as a library. As used herein, the term “library” means a collection of molecules. A library can contain a few or a large number of different molecules, varying from about ten molecules to several billion molecules or more. A molecule can comprise a naturally occurring molecule, a recombinant molecule, or a synthetic molecule. A plurality of test substances in a library can be assayed simultaneously. Optionally, test substances derived from different libraries can be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 6,180,348 and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that can potentially bind to a SLC26 polypeptide (e.g., U.S. Pat. Nos. 5,948,635, 5,747,334, and 5,498,538).

A library can comprise a random collection of molecules. Alternatively, a library can comprise a collection of molecules having a bias for a particular sequence, structure, or conformation. See e.g., U.S. Pat. Nos. 5,264,563 and 5,824,483. Methods for preparing libraries containing diverse populations of various types of molecules are known in the art, for example as described in U.S. Patents cited herein above. Numerous libraries are also commercially available.

VII.B. Expression Assays

The present invention also provides a method for identifying a substance that regulates SLCA26A6 gene expression. The term “gene expression” is used herein to refer generally to the cellular processes by which a functional SLC26 polypeptide is produced from a nucleic acid. Thus, a SLC26 modulator can comprise a substance that binds to and regulates a SLC26A6 promoter.

The term “promoter” refers to a nucleic acid that can direct gene expression of a nucleic acid to which it is operatively linked. A representative SLC26A6 promoter is set forth as SEQ ID NO:13.

Reporter Gene Assay. In one embodiment of the invention, a gene expression assay utilizes a chimeric gene that includes an isolated SLCA26A6 promoter region operably linked to a reporter gene. According to this method, a gene expression system is established that includes the chimeric gene and components required for gene transcription and translation so that reporter gene expression is assayable. To select a substance that modulates SLCA26A6 expression, the method further provides the steps of using the gene expression system to determine a baseline level of reporter gene expression in the absence of a test substance, providing a plurality of test substances to the gene expression system, and assaying a level of reporter gene expression in the presence of a test substance. A test substance is selected whose presence results in an altered level of reporter gene expression when compared to the baseline level.

To perform the disclosed method, the present invention further provides a chimeric gene comprising a SLCA26A6 promoter region operably linked to a heterologous nucleotide sequence. Preferably, the SLCA26A6 promoter region comprises the nucleic acid molecule of SEQ ID NO:13, or functional portion thereof. In a preferred embodiment, a chimeric gene of the invention is carried in a vector and expressed in a host cell. Preferred host cells include mammalian cells, for example HeLa cells.

The terms “reporter gene,” “marker gene,” and “selectable marker” each refer to a heterologous gene encoding a product that is readily observed and/or quantitated. Non-limiting examples of detectable reporter genes that can be operatively linked to a transcriptional regulatory region can be found in Alam & Cook (1990) Anal Biochem 188:245-254 and in PCT International Publication No. WO 97/47763. Preferred reporter genes for transcriptional analyses include the lacZ gene (Rose & Botstein, 1983), Green Fluorescent Protein (GFP) (Cubitt et al., 1995), luciferase, or chloramphenicol acetyl transferase (CAT).

An amount of reporter gene can be assayed by any method for qualitatively or preferably, quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct.

Representative methods for reporter gene assays can be found in U.S. Pat. No. 6,087,111, among other places.

One-Hybrid Analysis. Modulators that bind a SLC26A6 promoter can also be identified using one-hybrid analysis. According to this approach, a SLC26A6 promoter is operatively linked to one, or typically more, yeast reporter genes such as the lacZ gene, the URA3 gene, the LEU2 gene, the HIS3 gene, or the LYS2 gene, and the reporter gene fusion construct(s) is inserted into an appropriate yeast host strain. It is expected that the reporter genes are not transcriptionally active in the engineered yeast host strain, for lack of a transcriptional activator protein to bind the SLC26A6 promoter. The engineered yeast host strain is transformed with a library of cDNAs inserted in a yeast activation domain fusion protein expression vector, e.g. pGAD, where the coding regions of the cDNA inserts are fused to a functional yeast activation domain coding segment, such as those derived from the GAL4 or VP16 activators. Transformed yeast cells that acquire a cDNA encoding a protein that binds a cis-regulatory element of a SLC26A6 promoter can be identified based on the concerted activation the reporter genes, either by genetic selection for prototrophy (e.g., LEU2, HIS3, or LYS2 reporters) or by screening with chromogenic substrates (lacZ reporter gene) by methods known in the art. See e.g., Luo et al. (1996) Biotechniques 20:564-568; Vidal et al. (1996) Proc Natl Acad Sci USA 93:10315-10320; and Li & Herskowitz (1993) Science 262:1870-1874.

In Situ Filter Detection. About 10⁷ λgt11 clones of a cDNA expression library are prepared poly(A)⁺ RNA derived from a tissue where SLC26A6 is normally expressed (e.g., kidney). Clones are plated and replicated on nitrocellulose filters. After denaturation and renaturation, the filter-bound proteins are screened with a concatenated oligonucleotide probe containing the nucleotide sequence of a SLC26A6 promoter. The probe is prepared by nick translation with a specific activity of >10⁸/mg. Duplicate screening using a probe carrying a mutated SLC26A6 promoter is carried out to eliminate false positive clones.

VII.C. Binding Assays

In another embodiment, a method for identifying of a SLC26 modulator comprises determining specific binding of a test substance to a SLC26 polypeptide. The term “binding” refers to an affinity between two molecules. Preferably, specific binding also encompasses a quality or state of mutual action such that an activity of one protein or compound on another protein is inhibitory (in the case of an antagonist) or enhancing (in the case of an agonist).

The phrase “specifically (or selectively) binds”, when referring to the binding capacity of a candidate modulator, refers to a binding reaction which is determinative of the presence of the protein in a heterogeneous population of proteins and other biological materials. The binding of a modulator to a SLC26 polypeptide can be considered specific if the binding affinity is about 1×10⁴M⁻¹ to about 1×10⁶M⁻¹ or greater. The phrase “specifically binds” also refers to saturable binding. To demonstrate saturable binding of a test substance to a SLC26 polypeptide, Scatchard analysis can be carried out as described, for example, by Mak et al. (1989) J Biol Chem 264:21613-21618.

The phases “substantially lack binding” or “substantially no binding”, as used herein to describe binding of a modulator to a control polypeptide or sample, refers to a level of binding that encompasses non-specific or background binding, but does not include specific binding.

Several techniques can be used to detect interactions between a SLC26 polypeptide and a test substance without employing a known competitive modulator. Representative methods include, but are not limited to, Fluorescence Correlation Spectroscopy, Surface-Enhanced Laser Desorption/Ionization Time-Of-flight Spectroscopy, and Biacore technology, each technique described herein below. These methods are amenable to automated, high-throughput screening.

Fluorescence Correlation Spectroscopy. Fluorescence Correlation Spectroscopy (FCS) measures the average diffusion rate of a fluorescent molecule within a small sample volume (Tallgren, 1980). The sample size can be as low as 10³ fluorescent molecules and the sample volume as low as the cytoplasm of a single bacterium. The diffusion rate is a function of the mass of the molecule and decreases as the mass increases. FCS can therefore be applied to polypeptide-ligand interaction analysis by measuring the change in mass and therefore in diffusion rate of a molecule upon binding. In a typical experiment, the target to be analyzed (e.g., a SLC26 polypeptide) is expressed as a recombinant polypeptide with a sequence tag, such as a poly-histidine sequence, inserted at the N-terminus or C-terminus. The expression is mediated in a host cell, such as E. coli, yeast, Xenopus oocytes, or mammalian cells. The polypeptide is purified using chromatographic methods. For example, the poly-histidine tag can be used to bind the expressed polypeptide to a metal chelate column such as Ni²⁺ chelated on iminodiacetic acid agarose. The polypeptide is then labeled with a fluorescent tag such as carboxytetramethylrhodamine or BODIPY™ reagent (available from Molecular Probes of Eugene, Oreg.). The polypeptide is then exposed in solution to the potential ligand, and its diffusion rate is determined by FCS using instrumentation available from Carl Zeiss, Inc. (Thornwood, N.Y.). Ligand binding is determined by changes in the diffusion rate of the polypeptide.

Surface-Enhanced Laser DesorDtion/lonization. Surface-Enhanced Laser Desorption/lonization (SELDI) was developed by Hutchens & Yip (1993) Rapid Commun Mass Spectrom 7:576-580. When coupled to a time-of-flight mass spectrometer (TOF), SELDI provides a technique to rapidly analyze molecules retained on a chip. It can be applied to ligand-protein interaction analysis by covalently binding the target protein, or portion thereof, on the chip and analyzing by mass spectrometry the small molecules that bind to this protein (Worrall et al., 1998). In a typical experiment, a target polypeptide (e.g., a SLC26 polypeptide) is recombinantly expressed and purified. The target polypeptide is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target polypeptide are identified by the stringency of the wash needed to elute them.

Biacore. Biacore relies on changes in the refractive index at the surface layer upon binding of a ligand to a target polypeptide (e.g., a SLC26 polypeptide) immobilized on the layer. In this system, a collection of small ligands is injected sequentially in a 2-5 microliter cell, wherein the target polypeptide is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al., 1983; Malmquist, 1993). In a typical experiment, a target protein is recombinantly expressed, purified, and bound to a Biacore chip. Binding can be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al. (1999) Sensors and Actuators 54:3-15 and references therein.

VII.D. Conformational Assay

The present invention also provides a method for identifying a SLC26 modulator that relies on a conformational change of a SLC26 polypeptide when bound by or otherwise interacting with a SLC26 modulator.

Application of circular dichroism to solutions of macromolecules reveals the conformational states of these macromolecules. The technique can distinguish random coil, alpha helix, and beta chain conformational states.

To identify modulators of SLC26A, circular dichroism analysis can be performed using recombinantly expressed SLC26A. A SLC26 polypeptide is purified, for example by ion exchange and size exclusion chromatography, and mixed with a test substance. The mixture is subjected to circular dichroism. The conformation of a SLC26 polypeptide in the presence of a test substance is compared to a conformation of a SLC26 polypeptide in the absence of a test substance. A change in conformational state of a SLC26 polypeptide in the presence of a test substance can thus be used to identify a SLC26 modulator. Representative methods are described in U.S. Pat. Nos. 5,776,859 and 5,780,242.

VII.E. Anion Transport Assays

In a preferred embodiment of the invention, a method for identifying a SLC26 modulator employs a functional SLC26 polypeptide. Novel functional SLC26 polypeptides disclosed herein include any of SEQ ID NOs:2, 6, 8, and 10. Representative methods for determining anion transport activity of a functional SLC26 modulator include measuring anion flux and determining electrogenic transport, each described briefly herein below.

In accordance with the method, cells expressing SLC26 can be provided in the form of a kit useful for performing an assay of SLC26 function. Thus, cells can be frozen as described herein above and transported while frozen to others for performance of an assay. For example, in one embodiment of the invention, a test kit is provided for detecting a SLC26 modulator, the kit comprising: (a) frozen cells transfected with DNA encoding a full-length SLC26 polypeptide; and (b) a medium for growing the cells.

Preferably, a cell used in such an assay comprises a cell that is substantially devoid of native SLC26 and polypeptides substantially similar to SLC26. A preferred cell comprises a vertebrate cell, for example a Xenopus oocyte. In one embodiment of the invention, a cell used in the assay comprises a stable cell line that recombinantly expresses SLC26. Alternatively, a cell used in the assay can transiently express a SLC26 polypeptide as described in Example 5.

The term “substantially devoid of”, as used herein to describe a host cell or a control cell, refers to a quality of having a level of native SLC26A, a level of a polypeptide substantially similar to SLC26A, or a level of activity thereof, comprising a background level. The term “background level” encompasses non-specific measurements of expression or activity that are typically detected in a cell free of SLC26 and free of polypeptides substantially similar to SLC26A.

Also preferably, all assays employing cells expressing recombinant SLC26 additionally employ control cells that are substantially devoid of native SLC26 and polypeptides substantially similar to SLC26A. When using transiently transfected cells, a control cell can comprise, for example, an untransfected host cell. When using a stable cell line expressing SLC26A, a control cell can comprise, for example, a parent cell line used to derive the SLC26A-expressing cell line.

Assays of SLC26 activity that employ transiently transfected cells preferably include a marker that distinguishes transfected cells from non-transfected cells. The term “marker” refers to any detectable molecule that can be used to distinguish a cell that recombinantly expresses SLC26 from a cell that does not recombinantly express a SLC26 polypeptide. Preferably, a marker is encoded by or otherwise associated with a construct for SLC26 expression, such that cells are simultaneously transfected with a nucleic acid molecule encoding SLC26 and the marker. Representative detectable molecules that are useful as markers include but are not limited to a heterologous nucleic acid, a polypeptide encoded by a transfected construct (e.g., an enzyme or a fluorescent polypeptide), a binding protein, and an antigen.

A marker comprising a heterologous nucleic acid includes nucleic acids encoding a SLC26 polypeptide. Alternatively, any suitable method can be used to detect the encoded SLC26 polypeptide, as described herein below.

Examples of enzymes that are useful as markers include phosphatases (such as acid or alkaline phosphatase), β-galactosidase, urease, glucose oxidase, carbonic anhydrase, acetylcholinesterase, glucoamylase, maleate dehydrogenase, glucose-6-phosphate dehydrogenase, β-glucosidase, proteases, pyruvate decarboxylase, esterases, luciferase, alcohol dehydrogenase, or peroxidases (such as horseradish peroxidase).

A marker comprising an enzyme can be detected based on activity of the enzyme. Thus, a substrate is be added to catalyze a reaction the end product of which is detectable, for example using spectrophotometer, a luminometer, or a fluorimeter. Substrates for reaction by the above-mentioned enzymes, and that produce a detectable reaction product, are known to one of skill in the art.

A preferred marker comprises an encoded polypeptide that can be detected in the absence of an added substrate. Representative polypeptides that can be detected directly include GFP and EGFP. Common research equipment has been developed to perform high-throughput detection of fluorescence, for example GFP or EGFP fluorescence, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), and Genomic Solutions Inc. (Ann Arbor, Mich., United States of America). Most of the commercial systems use some form of scanning technology with photomultiplier tube detection.

Anion Flux Assay. A candidate substance can be tested for its ability to modulate a SLC26 polypeptide by determining anion flux across a membrane or lipid bilayer. Anion levels can be determined by any suitable approach. For example, an anion can be detected using a radiolabeled anion as described in Example 6.

Anion flux can also be measured using any of a variety of indicator compounds. Preferably, an indicator compound comprises a compound that can be detected in a high-throughput capacity. Representative fluorescent indicators useful for detecting halides (e.g., chloride) include quinolium-type Cl⁻ indicators (Verkman, 1990; Mansoura et al., 1999), cell-permeable indicators (Biwersi & Verkman, 1991), ratiometric indicators (Biwersi & Verkman, 1991), and long wavelength indicators (Biwersi et al., 1994; Jayaraman et al., 1999). An indicator can also comprise a recombinant protein. For example, the yellow fluorescent protein mutant, YFP-H148Q, produces fluorescence that is decreased upon halide binding (Jayaraman et al., 2000; Galietta et al., 2001). Such indicators are compatible with high-throughput assay formats and can be detected using, for example, an instrument for fluorescent detection as noted herein above.

Anion flux in a population of cultured cells can also be measured based on changes in a degree of light scattering that is correlated with cell size. See e.g., Krick et al. (1998) Pflugers Arch 435:415-421.

An anion flux assay can also comprise a competitive assay design. For example, the method can comprise: (a) providing an expression system, whereby a functional SLC26 polypeptide is expressed; (b) adding a SLC26 activator to the expression system, whereby anion transport is elicited; (c) adding a test substance to the expression system; and (d) observing a suppression of the anion transport in the presence of the SLC26 activator and the test substance, whereby an inhibitor of SLC26 is determined. Optionally, the persistent activator and test substance can be provided to the functional expression simultaneously. Similarly, an assay for determining a SLC26 activator can comprise steps (a)-(d) above with the exception that an enhancement of conductance is observed in the presence of the persistent activator and the test substance.

Electrogenic Transport Assay. Anion transport via a SLC26 polypeptide of the present invention can further be determined to be electrogenic by monitoring changes in intracellular pH (pHᵢ) or membrane voltage (Vₘ) during transport. Representative methods are described by Romero et al. (1998) Am J Physiol 274:F425-432 and Romero et al. (2000) J Biol Chem 275:24552-24559.

Briefly, an oocyte is visualized with a dissecting microscope and held on a nylon mesh in a chamber having a volume of about 250 μl. The oocyte is continuously superfused with a saline solution (3 ml/min to 5 ml/min) that is delivered through TYGON® tubing (Worchester, Mass., United States of America). Solutions can be switched using a daisy-chain system of computer-actuated five-way valves with zero dead space. Solution changes in the chamber typically occur within 15 seconds to about 20 seconds. Membrane voltage (Vₘ) and intracellular pH (pHᵢ) of X. laevis oocytes are measured simultaneously using microelectrodes, as described by Romero et al. (1997) Nature 387:409-413.

Vₘ electrodes can be pulled from borosilicate fiber-capillary glass (Warner Instruments of West Haven, Conn., United States of America). Electrodes are backfilled with 3M KCl and typically have a resistance of about 3MΩ to 5MΩ. The pH electrodes can be pulled in a similar manner, and are silanized by exposing them to 40 μl of bis-di-(methylamino)-dimethylsilane (Fluka Chemical of Ronkonkoma, N.Y., United States of America) for 5 minutes to 10 minutes. Silanized electrodes are deposited in an enclosed container at 200° C., and then baked overnight. The pH micropipettes are cooled under vacuum, and their tips are filled with hydrogen ionophore 1-cocktail B (Fluka Chemical of Ronkonkoma, N.Y., United States of America). The pH micropipettes are then backfilled with a buffer containing 0.04M KH₂PO₄, 0.023M NaOH, and 0.015M NaCl (pH 7.0). Representative pH microelectrodes have slopes ranging from about −54 mV/pH unit to −59 mV/pH unit.

The Vₘ and pHᵢ electrodes are connected to high-impedance electrometers as described by Davis et al. (1992) Am J Physiol 263:C246-256 and Siebens & Boron (1989) Am J Physiol 256:F354-365. The voltage due to pH can be obtained by electronically subtracting the signals from the pH and Vₘ electrodes. Vₘ can be obtained by subtracting the signals from the Vₘ electrode and an external reference (calomel) electrode.

In accordance with the methods of the present invention, electrogenic transport can be detected using any suitable method. For example, pH can also be assayed by detecting the presence of a fluorescence dye, for example BCECF (available from Photon Technology International, Inc. of Lawrenceville, N.J., United States of America).

Vesicle Transport Assays. Once a SLC26 modulator has been identified, its effectiveness in modulating anion transport activity can further be tested in isolated membrane vesicles, including brush border membrane vesicles derived from kidney and gut. Modulators can also be tested for activity in cultured grafts, for example intact renal proximal tubules. Methods for preparing membrane vesicles and exografts are known in the art, and representative protocols are described by Pritchard & Miller (1993) Physiol Rev 73:765-796; Miller et al. (1996) Am J Physiol 271:F508-520; Masereeuw et al. (1996) Am J Physiol 271:F1173-1182; Masereeuw et al. (1999) J Pharmacol Exp Ther 289:1104-1111; Hagenbuch et al. (1985) Pflugers Arch 405:202-208; Kuo & Aronson (1988) J Biol Chem 263:9710-9717; and Pritchard & Renfro (1983) Proc Natl Acad Sci USA 80:2603-2607.

VII.F. Rational Design

The knowledge of the structure a native SLC26 polypeptide provides an approach for rational design of modulators and diagnostic agents. In brief, the structure of a SLC26 polypeptide can be determined by X-ray crystallography and/or by computational algorithms that generate three-dimensional representations. See Saqi et al. (1999) Bioinformatics 15:521-522; Huang et al. (2000) Pac Symp Biocomput:230-241; and PCT International Publication No. WO 99/26966. Alternatively, a working model of a SLC26 polypeptide structure can be derived by homology modeling (Maalouf et al., 1998). Computer models can further predict binding of a protein structure to various substrate molecules that can be synthesized and tested using the assays described herein above. Additional compound design techniques are described in U.S. Pat. Nos. 5,834,228 and 5,872,011.

In general, a SLC26 polypeptide is a membrane protein, and can be purified in soluble form using detergents or other suitable amphiphilic molecules. The resulting SLC26 polypeptide is in sufficient purity and concentration for crystallization. The purified SLC26 polypeptide preferably runs as a single band under reducing or non-reducing polyacrylamide gel electrophoresis (PAGE). The purified SLC26 polypeptide can be crystallized under varying conditions of at least one of the following: pH, buffer type, buffer concentration, salt type, polymer type, polymer concentration, other precipitating ligands, and concentration of purified SLC26. Methods for generating a crystalline polypeptide are known in the art and can be reasonably adapted for determination of a SLC26 polypeptide as disclosed herein. See e.g., Deisenhofer et al. (1984) J Mol Biol 180:385-398; Weiss et al. (1990) FEBS Lett 267:268-272; or the methods provided in a commercial kit, such as the CRYSTAL SCREEN™ kit (available from Hampton Research of Riverside, Calif., United States of America).

A crystallized SLC26 polypeptide can be tested for functional activity and differently sized and shaped crystals are further tested for suitability in X-ray diffraction. Generally, larger crystals provide better crystallography than smaller crystals, and thicker crystals provide better crystallography than thinner crystals. Preferably, SLC26 crystals range in size from 0.1-1.5 mm. These crystals diffract X-rays to at least 10 Å resolution, such as 1.5-10.0 Å or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å or less being preferred for the highest resolution.

VIII. Methods for Detecting a SLC26 Polypeptide

The present invention further provides methods for detecting a SLC26 polypeptide. The disclosed methods can be used for determining altered levels of SLC26 expression that are associated with disorders and disease states, including but not limited to conditions of oxalate hyperexcretion (for example, in renal stone disease), CF-related and idiopathic pancreatitis, hypertension, edema, and other conditions of abnormal salt, oxalate, or bicarbonate transport.

In one embodiment of the invention, the method involves performing an immunochemical reaction with an antibody that specifically recognizes a SLC26 polypeptide, wherein the antibody was prepared according to a method of the present invention for producing such an antibody. Thus, the method comprises: (a) obtaining a biological sample comprising peptidic material; (b) contacting the biological sample with an antibody that specifically binds a SLC26 polypeptide and that was produced according to the disclosed methods, wherein the antibody comprises a detectable label; and (c) detecting the detectable label, whereby a SLC26 polypeptide in a sample is detected.

Techniques for detecting such antibody-antigen conjugates or complexes are known in the art and include but are not limited to centrifugation, affinity chromatography and other immunochemical methods. See e.g., Manson (1992) Immunochemical Protocols. Humana Press, Totowa, N.J., United States of America; Ishikawa (1999) Ultrasensitive and Rapid Enzyme Immunoassay. Elsevier, Amsterdam/N.Y., United States of America; Law (1996) Immunoassay: A Practical Guide. Taylor & Francis, London/Bristol, Pennsylvania, United States of America; Chan (1996) Immunoassay Automation: An Updated Guide to Systems. Academic Press, San Diego; Liddell & Weeks (1995) Antibody Technology. Bios Scientific Publishers, Oxford, United Kingdom; Masseyeff et al. (1993) Methods of Immunological Analysis. VCH Verlagsgesellschaft/VCH Publishers, Weinheim, Federal Republic of Germany/N.Y., United States of America; Walker & Rapley (1993) Molecular and Antibody Probes in Diagnosis. Wiley, Chichester, N.Y.; Wyckoff et al. (1985) Diffraction Methods for Biological Macromolecules. Academic Press, Orlando, Fla., United States of America; and references cited therein.

In another embodiment of the invention, a modulator that shows specific binding to a SLC26 polypeptide is used to detect a SLC26 anion transporter. Analogous to detection of a SLC26 polypeptide using an antibody, the method comprises: (a) obtaining a biological sample comprising peptidic material; (b) contacting the biological sample with a modulator of a SLC26 polypeptide, wherein the modulator comprises a detectable label; and (c) detecting the detectable label, whereby a SLC26 polypeptide in a sample is detected. Any suitable detectable label can be used, for example a fluorophore or epitope label.

IX. Therapeutic Applications

The present invention provides methods for identification of modulators of anion transport activity via SLC26A6, SLC26A1, and SLC26A2. Alternatively, a construct encoding a recombinant SLC26 polypeptide of the invention can be used to replace diminished or lost SLC26 function. The modulators and constructs of the invention are useful for regulation of anion transport in a subject, for example to remedy dysfunctional anion transport associated with sulphate homeostasis, sulphation, oxalate homeostasis, transepithelial salt transport, bicarbonate transport, and physiological pH regulation.

The term “subject” as used herein includes any vertebrate species, preferably warm-blooded vertebrates such as mammals and birds. More particularly, the methods of the present invention are contemplated for the treatment of tumors in mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economical importance to humans.

Functional characterization of SLC26A6, as disclosed herein, indicates that it can mediate Cl⁻-formate exchange, Cl⁻—Cl⁻ exchange, SO₄²⁻-exchange, Cl⁻-oxalate exchange, and Cl⁻—HCO₃⁻ exchange. The anion transport properties of SLC26A6 point to its role in a variety of physiological functions, including but not limited to regulation of sulphate homeostasis, oxalate secretion, transepithelial salt absorption, and bicarbonate transport, including CFTR-dependent bicarbonate transport, as described further herein below.

In one embodiment of the invention, modulators of SLC26A6 activity can be used for the treatment or prevention of conditions of disrupted anion transport. In another embodiment of the invention, modulators of SLC26A6 expression can be used to treat conditions or pathologies resulting from low levels of SLC26A6 expression and/or low levels of SLC26A6 activity. A modulator that enhances SLC26A6 expression can be identified using the methods disclosed herein above. Alternatively, a construct encoding a recombinant human SLC26A6 polypeptide can be used to replace diminished or lost SLC26A6 function.

The present invention also provides that SLC26A1 does not transport Cl⁻ or formate, but does transport SO₄²⁻ and oxalate. The anion transport activities of SLC26A1 are in close agreement with those found in renal proximal tubule basolateral membrane vesicles and are important for sulphate and oxalate homeostasis, as described further herein below. Thus, SLC26A1 modulators, identified by the methods of the present invention can also be used for the treatment or prevention of conditions of disrupted anion transport.

Also disclosed herein is the observation that transport of SO₄²⁻ and oxalate by SLC26A1 is strongly activated by impermeant, monovalent anions such as Cl⁻ and formate. Activation by impermeant monovalent anions appears not to be simply a matter of the valence of anionic charge, since HCO₃⁻ does not activate SO₄²⁻ uptake by rat SLC26A1 (Satoh et al., 1998). This feature of transport activation is dramatically different from that of other SLC26 anion exchangers, including the closely related transporter SLC26A2 (Satoh et al., 1998), and identifies a new approach for modulating SLC26A1 activity.

The present invention further provides novel observations of anion transport via SLC26A2, a sulphate transporter that contributes to normal sulphation of proteoglycans in bone. Thus, SLC26A2 modulators, including pH modifiers, can be used to modulate SLC26A2 activity to thereby facilitate bone health, and to treat or prevent bone disease, as described further herein below.

The present invention still further provides novel methods for modulating anion transport via SLC26A6 and SLC26A2 by regulating pH. In particular, acidification of a cellular environment can be used to selectively activate anion transport. For example, modulation of extracellular pH to about 6.0 can be used to activate Cl⁻—HCO₃⁻ exchange by SLC26A6. SO₄²⁻ transport via SLC26A2 is also strongly activated by an acid-outside pH gradient, particularly in the presence of extracellular Cl⁻.

IX.A. Sulphate Homeostasis

The homeostasis of inorganic sulphate, a physiological anion that is utilized in conjugation reactions of exogenous and endogenous compounds, is maintained by intracellular hydrolysis of sulfoconjugates, oxidation of reduced organic sulfur, and transport of sulphate from extracellular fluids. Sulphate absorption from the gastrointestinal tract and reabsorption by the renal tubules are critical mechanisms for maintenance of sulphate levels.

Sulphate uptake is initiated across the brush-border membrane (BBM) by sodium-dependent sulphate cotransport driven by a lumenal membrane sodium gradient. For a review, see Beck & Silve (2001) Kidney Int 59:835-845. In addition to the sodium-dependent cotransport system, studies in BBM vesicles have suggested that sulphate can be transported by an anion exchange mechanism (Karniski & Aronson, 1987; Pritchard, 1987; Talor et al., 1987). The transport properties of the BBM sulphate/anion transporter are in close agreement with those of SLC26A6, as disclosed herein.

Exit of sulphate across the basolateral membrane (BLM) occurs via a sulphate anion exchanger, which completes the process of transcellular sulphate reabsorption in the proximal tubule. Sulphate transport across the BLM can utilize hydroxyl ions, bicarbonate, and oxalate as counterions (Low et al., 1984; Pritchard, 1987). These transport characteristics are in close agreement with those displayed by SLC26A1, as disclosed herein.

Slight imbalances in sulphate homeostasis can lead to clinical manifestations, including hyposulphatemia, hypersulphatemia, and altered sulphate metabolism. Representative syndromes/disease in which the formation of sulphate ion or the metabolism of oxidized sulfur is disturbed include Hunter's syndrome, Morquio's syndrome, Maroteaux-Lamy syndrome, metachromatic leuokodystrophy, and multiple-sulfohydrolase deficiency (Tallgren, 1980).

Increased serum levels of sulphate (hypersulphatemia) are observed in patients suffering from chronic renal failure. Increased serum sulphate can alter the sulphation of many endogenous substances and hormones (Falany, 1997; Coughtrie et al., 1998). In most cases, the sulphation of these compounds leads to an increase in their urinary excretion (Falany, 1997). Excess sulphate can also lead to a reduction in ionized calcium, thereby contributing to the pathogenesis of renal osteodystrophy (Michalk et al., 1990).

Exogenous substances can also disrupt renal handling of sulphate. For example, chronic exposure of heavy metals (e.g., mercury, cadmium, lead, and chromium) can lead to nephrotoxicity and hepatotoxicity. Heavy metals can cause cellular necrosis as well as altered absorptive properties in the kidney, causing proteinuria, glucosuria, aminoaciduria, calciuria, phophaturia, and sulfaturia (Vacca et al., 1986; Miura et al., 2000). In particular, maximal sulphate transport via SLC26A1 is strongly inhibited by mercury (Markovich & James, 1999).

The properties of SO₄²⁻ transport via SLC26A6, as disclosed herein, are also similar to those described in placenta (Grassl, 1996), lung (Mohapatra et al., 1993), and pancreas (Elgavish & Meezan, 1992). SLC26A6 is expressed in each of these tissues, suggesting that SLC26A6 can mediate the observed transport. SLC26A1 activity has also been demonstrated in brain, where it is proposed to contribute to myelin sulphation (Lee et al., 1999a).

IX.B. Oxalate Homeostasis

The ability of SLC26A1 and SLC26A6 to mediate oxalate exchange suggests important roles for these transporters in oxalate homeostasis. Apical oxalate transport by a DIDS-sensitive anion transporter functions in concert with basolateral oxalate transport mediated by SLC26A1 (Sat-1) to secrete oxalate from the proximal tubule (Senekjian & Weinman, 1982). Of note, sulphate transport by rat DTDST is cis-inhibited by oxalate, consistent with oxalate transport by this SLC26 protein (Satoh, 1998).

Similar data has been reported for SLC26A3 (DRA), although the absolute value of heterologous expression was extremely low (Moseley, 1999). SLC26A2 and SLC26A3 are also expressed in the intestine (Haila, 2000; Haila, 2001; Silberg, 1995), where they can play a role in intestinal oxalate transport.

SLC26A6 is expressed at the apical membrane of the proximal tubule (Knauf et al., 2001). The anion transport properties of SLC26A6, disclosed herein, are consistent with the role of SLC26A6 as the apical renal oxalate transporter. As disclosed herein, SLC26A6 is strongly expressed in small intestine, and thus can also mediate oxalate transport in gut.

Hyper-excretion of oxalate is an important factor in the pathogenesis of renal stones, and increased red cell oxalate transport has been shown to segregate with oxalate excretion in kindreds with nephrolithiasis (Baggio et al., 1986). Dietary absorption of oxalate is an important determinant of urinary excretion (Holmes et al., 2001).

Based on the foregoing evidence, variation in the human SLC26A6 gene is implicated as a risk factor for nephrolithiasis. Similarly, modulators of SLC26A6 and SLC26A1, identified as disclosed herein, can be used to treat and/or prevent nephrolithiasis.

IX.C. Transepithelial Sodium Absorption

Na⁺—H⁺ exchange mediated by NHE-3 (Na⁺—H⁺ exchanging protein 3) functions in conjunction with apical chloride-formate exchange to mediate transepithelial reabsorption of Na⁺—Cl⁻ by the kidney proximal tubule (Wang et al., 2001) and by segments of the distal nephron (Wang et al., 1992). Apical Cl⁻-base exchange in renal vesicle and whole tubule preparations is also implicated in transepithelial Na⁺—Cl⁻ absorption by the proximal tubule (Kurtz et al., 1994). Transcellular NaCl transport in this nephron segment is believed to be mediated by the concerted action of an apical Na⁺/H⁺ exchanger and a Cl⁻/OH⁻ exchanger to secrete H⁺ and OH⁻ ions that form H₂O in the tubule lumen.

SLC26A6 protein is detected at the apical membrane of epithelial cells (Lohi et al., 2000), including those of the kidney proximal tubule (Knauf et al., 2001). Based on these observations, SLC26A6 was proposed to be the Cl⁻-formate exchanger of the kidney proximal tubule.

The present invention discloses that SLC26A6 mediates Cl⁻-formate exchange and can thereby mediate transepithelial salt reabsorption in the proximal tubule. The present invention still further provides that SLC26A6 can mediate transepithelial salt exchange by Cl⁻-base (Cl⁻—OH and/or Cl⁻—HCO₃⁻) exchange. Thus, modulators of SLC26A6, identified as disclosed herein, are useful for the treatment or prevention of Na⁺ absorption.

IX.D. Duodenal Ulcer Disease

Protection of duodenal epithelial cells from lumenal acid is mediated by several mechanisms including regulation of intracellular pH and secretion of bicarbonate from the pancreas and Brunner's glands. In the absence of such protection, duodenal cells and other cells of the upper gastrointestinal tract are believed to reversibly acidify in the presence of acidic lumenal contents, thereby injuring the epithelium. Lumenal acid also upregulates other putative defense mechanisms, such as mucosal blood flow and mucus gel secretion, suggesting that regulation of bicarbonate levels is part of a multi-component defensive system. See Akiba et al. (2001) J Clin Invest 108:1807-1816; Flemstrom & Isenberg (2001) News Physiol Sci 16:23-28; and references cited therein.

The present invention provides that SLC26A6 can mediate Cl⁻-base (Cl⁻—OH and/or Cl⁻—HCO₃⁻) exchange. SLC26A6 protein is detected in duodenum (Wang, 2002), consistent with a role for SLC26A6 in Cl⁻—HCO₃⁻) exchange in gut. Thus, modulators of SLC26A6 can be used to regulate acid levels in the gut to thereby treat or prevent conditions such as duodenal ulcer disease.

IX.E. Cystic Fibrosis

The role of SLC26A6 in Cl⁻—HCO₃⁻ exchange is also relevant to the physiology of tissues that excrete HCO₃⁻ under the influence of CFTR, a chloride channel whose dysfunction results in cystic fibrosis. In particular, Cl⁻-base exchange by SLC26A6 is characteristic of the apical CFTR-dependent bicarbonate transporter in lung (Lee et al., 1998), submandibular gland (Lee et al., 1999b), and exocrine pancreas (Lee et al., 1999b; Choi et al., 2001). Studies of cystic fibrosis pancreatic cell lines have shown that expression of wild type CFTR can elicit an increase in SLC26A6 transcripts, a 10-fold activation of DIDS-sensitive sulphate transport, and elevated levels of Cl⁻—HCO₃⁻ exchange (Elgavish & Meezan, 1992; Greeley et al., 2001). In addition, the inability of CFTR mutants to regulate Cl⁻—HCO₃⁻ exchange is correlated with the pancreatic insufficiency (Choi et al., 2001).

The present invention provides that SLC26A6 can mediate Cl⁻-base (Cl⁻—OH and/or Cl⁻—HCO₃⁻) exchange. Thus, modulators of SLC26A6 can be used to activate Cl⁻—HCO₃⁻ exchange in CF patients.

IX.F. Chondrodysplasia

SLC26A2 (also called DTDS™) encodes an anion transporter whose abnormal function can result in any one of several chondrodysplasias, including diastrophic dysplasia, astelogenesis type 2, achrondrogenesis type 1B, and multiple ephiphyseal dysplasia (Hastbacka et al., 1994; Superti-Furga et al., 1996; Newbury-Ecob, 1998). Biochemical studies of patients with these disorders revealed defects in sulphate uptake, the presence of undersulphated proteoglycans, and a reduced rate of sulphate incorporation into chondroitin sulphate. In addition, more significantly reduced levels of proteoglycan sulphation are correlated with clinical severity. See Everett & Green (1999) Hum Mol Genet 8:1883-1891 and references cited therein. Thus clinical strategies for the treatment of chondroplasia can be directed toward restoring sulphate transport mediated by SLC26A2 to normal levels. Thus, modulators of SLC26A2, in particular pH modifiers, can be used to prevent or to treat bone disorders associated with poor proteoglycan sulphation.

X. Compositions and Therapeutic Methods

In accordance with the methods of the present invention, a composition that is administered to alter anion transport activity in a subject comprises: (a) an effective amount of a SLC26 modulator; and (b) a pharmaceutically acceptable carrier. A SLC26 modulator can comprise any one of the types of test substances described herein above. A SLC26 modulator can also comprise a pH modifier.

The present invention also provides methods for modulating anion transport activity in a subject via administration of a gene therapy construct comprising an SLC26 polypeptide. Such a construct can be prepared as described herein above, further comprising a carrier suitable for administration to a subject.

X.A. pH Modifiers

In one embodiment of the invention, a method is provided for modulating SLC26 anion transport by altering pH. The disclosure of the present invention shows that Cl⁻—HCO₃⁻ exchange via SLC26A6 is activated by an acid-outside environment, for example an extracellular pH of about 6. SO₄²⁻ transport via SLC26A2 is similarly activated by an acid-outside pH. Thus, the present invention provides a method for activating anion transport in a subject, the method comprising administering a modulator of a SLC26 polypeptide to the subject, wherein the modulator comprises a pH modifier.

The term “pH modifier” refers to any substance that can be used to regulate the pH of an in situ environment. An effective amount of a pH modifier comprises an amount sufficient to alter a pH to a level sufficient for activation of a SLC26 polypeptide. An effective amount of a pH modifier effective to achieve the desired in vivo pH modification will depend on the acidity or basicity (pKa or pKb) of the compound used, the pH of the carrier (e.g., a polymer composition) used when in vivo, and the in vivo environment's physiologic pH.

Representative pH modifiers include acidic compounds or anhydrous precursors thereof, or chemically protected acids. For example, a pH modifier can comprise at least one member selected from the group consisting of: amino acids; carboxylic acids and salts thereof; di-acids and salts thereof; poly-acids and salts thereof; esters that are easily hydrolyzable in vivo; lactones that are easily hydrolyzable in viva; organic carbonates; enolic compounds; acidic phenols; polyphenolic compounds; aromatic alcohols; ammonium compounds or salts thereof; boron-containing compounds; sulfonic acids and salts thereof; sulfinic acids and salts thereof; phosphorus-containing compounds; acid halides; chloroformates; acid gases; acid anhydrides; inorganic acids and salts thereof; and polymers having functional groups of at least one of the preceding members. A pH modifier of this invention can also comprise at least one member selected from the group consisting of: glycine; alanine; proline; lysine; glutaric acid; D-galacturonic acid; succinic acid; lactic acid; glycolic acid; poly(acrylic acid); sodium acetate; diglycolic anhydride; succinic anhydride; citraconic anhydride; maleic anhydride; lactide; diethyl oxalate; Meldrum's acid; diethyl carbonate; dipropyl carbonate; diethyl pyrocarbonate; diallyl pyrocarbonate; di-tert-butyl dicarbonate; ascorbic acid; catechin; ammonium chloride; D-glucosamine hydrochloride; 4-hydroxy-ephedrine hydrochloride; boric acid; nitric acid; hydrochloric acid; sulfuric acid; ethanesulfonic acid; and p-toluenesulfonic acid; 2-aminoethylphosphoric acid; methylphosphonic acid; dimethylphosphinic acid; methyl chloroformate; sulfur dioxide; and carbon dioxide.

A pH modifier can be prepared in a micorcapsule, such that the pH modifier diffuses through the microcapsule or is released by bioerosion of the microcapsule. The microcapsule may be formulated so that the pH modifier is released from the microcapsule continuously over a period of time. Microencapsulation of the pH modifier can be achieved by many known microencapsulation techniques, as described further herein below under the heading “Carriers.”

X.B. Carriers

Any suitable carrier that facilitates preparation and/or administration of a SLC26 modulator can be used. The carrier can be a viral vector or a non-viral vector. Suitable viral vectors include adenoviruses, adeno-associated viruses (AAVs), retroviruses, pseudotyped retroviruses, herpes viruses, vaccinia viruses, Semiliki forest virus, and baculoviruses.

Suitable non-viral vectors that can be used to deliver a SLC26 polypeptide or a SLC26 modulator include but are not limited to a plasmid, a nanosphere (Manome et al., 1994; Saltzman & Fung, 1997), a peptide (U.S. Pat. Nos. 6,127,339 and 5,574,172), a glycosaminoglycan (U.S. Pat. No. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997) and U.S. Pat. Nos. 4,551,482, 5,714,166, 5,510,103, 5,490,840, and 5,855,900), and a polysome (U.S. Pat. No. 5,922,545).

Where appropriate, two or more types of carriers can be used together. For example, a plasmid vector can be used in conjunction with liposomes.

A carrier can be selected to effect sustained bioavailability of a SLC26 modulator to a site in need of treatment. The term “sustained bioavailability” encompasses factors including but not limited to prolonged release of a SLC26 modulator from a carrier, metabolic stability of a SLC26 modulator, systemic transport of a composition comprising a SLC26 modulator, and effective dose of a SLC26 modulator.

Representative compositions for sustained bioavailability can include but are not limited to polymer matrices, including swelling and biodegradable polymer matrices, (U.S. Pat. Nos. 6,335,035; 6,312,713; 6,296,842; 6,287,587; 6,267,981; 6,262,127; and 6,221,958), polymer-coated microparticles (U.S. Pat. Nos. 6,120,787 and 6,090,925) a polyol:oil suspension (U.S. Pat. No. 6,245,740), porous particles (U.S. Pat. No. 6,238,705), latex/wax coated granules (U.S. Pat. No. 6,238,704), chitosan microcapsules, and microsphere emulsions (U.S. Pat. No. 6,190,700).

Microcapsules. Microencapsulation can be carried out by dissolving a coating polymer in a volatile solvent, e.g., methylene chloride, to a polymer concentration of about 6% by weight; adding a pH modifying compound (selected to be acidic or basic according to the pH level to be achieved in situ) in particulate form to the coating polymer/solvent solution under agitation, to yield a pH modifier concentration of 2% to 10% by weight; adding the resulting polymer dispersion to a methylene chloride solution containing a phase inducer, such as silicone oil, under agitation; allowing the mixture to equilibrate for about 20 minutes; further adding the mixture slowly to a non-solvent, such as heptane, under rapid agitation; allowing the more volatile solvent to evaporate under agitation; removing the agitator; separating the solids from the silicone oil and heptane; and washing and drying the microcapsules. The size of the microcapusles will range from about 0.001 to about 1000 microns. See e.g., U.S. Pat. No. 6,061,581.

A microencapsulating coating polymer is preferably biodegradable and/or can permit diffusion of the encapsulated modulator (e.g., a pH modifier). A microencapsulating coating also preferably has low inherent moisture content. Biodegradation preferably occurs at rates greater than or similar to the rate of degradation of the base polymer.

Examples of coating materials that can be used to microencapsulate a SLC26 modulator, for example a pH modifier, include but are not limited to polyesters, such as polyglycolic acid, polylactic acid, copolymers of polyglycolic acid and polylactic acid, polycaprolactone, poly-β-hydroxybutyrate, copolymers of ε-caprolactone and δ-valerolactone, copolymers of ε-caprolactone and DL-dilactide, and polyester hydrogels; polyvinylpyrrolidone; polyamides; gelatin; albumin; proteins; collagen; poly(orthoesters); poly(anhydrides); poly(alkyl-2-cyanoacrylates); poly(dihydropyrans); poly(acetals); poly(phosphazenes); poly(urethanes); poly(dioxinones); cellulose; and starches.

Viral Gene Therapy Vectors. Viral vectors of the invention are preferably disabled, e.g. replication-deficient. That is, they lack one or more functional genes required for their replication, which prevents their uncontrolled replication in vivo and avoids undesirable side effects of viral infection. Preferably, all of the viral genome is removed except for the minimum genomic elements required to package the viral genome incorporating the therapeutic gene into the viral coat or capsid. For example, it is desirable to delete all the viral genome except: (a) the Long Terminal Repeats (LTRs) or Invented Terminal Repeats (ITRs); and (b) a packaging signal. In the case of adenoviruses, deletions are typically made in the E1 region and optionally in one or more of the E2, E3 and/or E4 regions. Other viral vectors can be similarly deleted of genes required for replication. Deletion of sequences can be achieved by a recombinant approach, for example, involving digestion with appropriate restriction enzymes, followed by re-ligation. Replication-competent self-limiting or self-destructing viral vectors can also be used.

Nucleic acid constructs of the invention can be incorporated into viral genomes by any suitable approach known in the art. Typically, such incorporation is performed by ligating the construct into an appropriate restriction site in the genome of the virus. Viral genomes can then be packaged into viral coats or capsids using any suitable procedure. In particular, any suitable packaging cell line can be used to generate viral vectors of the invention. These packaging lines complement the replication-deficient viral genomes of the invention, as they include, for example by incorporation into their genomes, the genes that have been deleted from the replication-deficient genome. Thus, the use of packaging lines allows viral vectors of the invention to be generated in culture.

Suitable packaging lines for retroviruses include derivatives of PA317 cells, ψ-2 cells, CRE cells, CRIP cells, E-86-GP cells, and 293GP cells. Line 293 cells are preferred for use with adenoviruses and adeno-associated viruses.

Plasmid Gene Therapy Vectors. A SLC26 modulator or SLC26 polypeptide can also be encoded by a plasmid. Advantages of a plasmid carrier include low toxicity and easy large-scale production. A polymer-coated plasmid can be delivered using electroporation as described by Fewell et al. (2001) Mol Ther 3:574-583. Alternatively, a plasmid can be combined with an additional carrier, for example a cationic polyamine, a dendrimer, or a lipid, that facilitates delivery. See e.g., Baher et al. (1999) Anticancer Res 19:2917-2924; Maruyama-Tabata et al. (2000) Gene Ther 7:53-60; and Tam et al. (2000) Gene Ther 7:1867-1874.

Liposomes. A composition of the invention can also be delivered using a liposome. Liposomes can be prepared by any of a variety of techniques that are known in the art. See e.g., - - - (1997). Current Protocols in Human Genetics on CD-ROM. John Wiley & Sons, New York; Lasic & Martin (1995) STEALTH® Liposomes. CRC Press, Boca Raton, Fla., United States of America; Janoff (1999) Liposomes: Rational Design. M. Dekker, New York; Gregoriadis (1993) Liposome Technology, 2nd ed. CRC Press, Boca Raton, Fla., United States of America; Betageri et al. (1993) Liposome Drug Delivery Systems. Technomic Pub., Lancaster; Pa., United States of America.; and U.S. Pat. Nos. 4,235,871; 4,551,482; 6,197,333; and 6,132,766. Temperature-sensitive liposomes can also be used, for example THERMOSOMES™ as disclosed in U.S. Pat. No. 6,200,598. Entrapment of a SLC26 modulator or a SLC26 polypeptide within liposomes of the present invention can be carried out using any conventional method in the art. In preparing liposome compositions, stabilizers such as antioxidants and other additives can be used.

Other lipid carriers can also be used in accordance with the claimed invention, such as lipid microparticles, micelles, lipid suspensions, and lipid emulsions. See e.g., Labat-Moleur et al. (1996) Gene Therapy 3:1010-1017; and U.S. Pat. Nos. 5,011,634; 6,056,938; 6,217,886; 5,948,767; and 6,210,707.

X.B. Targeting Ligands

As desired, a composition of the invention can include one or more ligands having affinity for a specific cellular marker to thereby enhance delivery of a SLC26 modulator or a SLC26 polypeptide to a site in need of treatment in a subject. Ligands include antibodies, cell surface markers, peptides, and the like, which act to home the therapeutic composition to particular cells.

The terms “targeting” and “homing”, as used herein to describe the in vivo activity of a ligand following administration to a subject, each refer to the preferential movement and/or accumulation of a ligand in a target tissue (e.g., a tumor) as compared with a control tissue.

The term “target tissue” as used herein refers to an intended site for accumulation of a ligand following administration to a subject. For example, the methods of the present invention employ a target tissue comprising a tumor. The term “control tissue” as used herein refers to a site suspected to substantially lack binding and/or accumulation of an administered ligand.

The terms “selective targeting” of “selective homing” as used herein each refer to a preferential localization of a ligand that results in an amount of ligand in a target tissue that is about 2-fold greater than an amount of ligand in a control tissue, more preferably an amount that is about 5-fold or greater, and most preferably an amount that is about 10-fold or greater. The terms “selective targeting” and “selective homing” also refer to binding or accumulation of a ligand in a target tissue concomitant with an absence of targeting to a control tissue, preferably the absence of targeting to all control tissues.

The terms “targeting ligand” and “targeting molecule” as used herein each refer to a ligand that displays targeting activity. Preferably, a targeting ligand displays selective targeting. Representative targeting ligands include peptides and antibodies.

The term “peptide” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics. Representative peptide ligands that show tumor-binding activity include, for example, those described in U.S. Pat. Nos. 6,180,084 and 6,296,832.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). See U.S. Pat. Nos. 5,111,867; 5,632,991; 5,849,877; 5,948,647; 6,054,561 and PCT International Publication No. WO 98/10795.

Antibodies, peptides, or other ligands can be coupled to drugs (e.g., a SLC26 modulator or a gene therapy construct comprising a SLC26 polypeptide) or drug carriers using methods known in the art, including but not limited to carbodiimide conjugation, esterification, sodium periodate oxidation followed by reductive alkylation, and glutaraldehyde crosslinking. See e.g., Bauminger & Wilchek (1980) Methods Enzymol 70:151-159;
Goldman et al. (1997) Cancer Res 57:1447-1451; Kirpotin et al. (1997) Biochemistry 36:66-75; - - - (1997). Current Protocols in Human Genetics on CD-ROM. John Wiley & Sons, New York; Neri et al. (1997) Nat Biotechnol 15:1271-1275; Park et al. (1997) Cancer Lett 118:153-160; and Pasqualini et al. (1997) Nat Biotechnol 15:542-546; U.S. Pat. No. 6,071,890; and European Patent No. 0 439 095. Alternatively, pseudotyping of a retrovirus can be used to target a virus towards a particular cell (Marin et al., 1997).

X.C. Formulation

Suitable formulations for administration of a composition of the invention to a subject include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some preferred ingredients are sodium dodecyl sulphate (SDS), for example in the range of 0.1 to 10 mg/ml, preferably about 2.0 mg/ml; and/or mannitol or another sugar, for example in the range of 10 to 100 mg/ml, preferably about 30 mg/ml; phosphate-buffered saline (PBS), and any other formulation agents conventional in the art.

The therapeutic regimens and compositions of the invention can be used with additional adjuvants or biological response modifiers including, but not limited to, the cytokines interferon alpha (IFN-α), interferon gamma (IFN-γ), interleukin 2 (IL2), interleukin 4 (IL4), interleukin 6 (IL6), tumor necrosis factor (TNF), or other cytokine affecting immune cells.

X.D. Dose and Administration

A composition of the present invention can be administered to a subject systemically, parenterally, or orally. The term “parenteral” as used herein includes intravenous injection, intramuscular injection, intra-arterial injection, and infusion techniques. For delivery of compositions to pulmonary pathways, compositions can be administered as an aerosol or coarse spray. A delivery method is selected based on considerations such as the type of the type of carrier or vector, therapeutic efficacy of the composition, and the condition to be treated.

Preferably, an effective amount of a composition of the invention is administered to a subject. For example, an “effective amount” is an amount of a composition sufficient to modulate SLC26 anion transport activity.

Actual dosage levels of active ingredients in a therapeutic composition of the invention can be varied so as to administer an amount of the composition that is effective to achieve the desired therapeutic response for a particular subject. The selected dosage level will depend upon a variety of factors including the activity of the therapeutic composition, formulation, the route of administration, combination with other drugs or treatments, the disease or disorder to be treated, and the physical condition and prior medical history of the subject being treated. Determination and adjustment of an effective amount or dose, as well as evaluation of when and how to make such adjustments, are known to those of ordinary skill in the art of medicine.

For local administration of viral vectors, previous clinical studies have demonstrated that up to 10¹³ pfu (plaque forming units) of virus can be injected with minimal toxicity. In human patients, 1×10⁹-1×10¹³ pfu are routinely used. See Habib et al. (1999) Hum Gene Ther 10:2019-2034. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize the desired modulation of anion transporter activity.

For soluble formulations of a composition of the present invention, conventional methods of extrapolating human dosage are based on doses administered to a murine animal model can be carried out using the conversion factor for converting the mouse dosage to human dosage: Dose Human per kg=Dose Mouse per kg×12 (Freireich et al., 1966). Drug doses are also given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/m² dose, the dose is multiplied by the appropriate km factor. In adult humans, 100 mg/kg is equivalent to 100 mg/kg×37 kg/m²=3700 mg/m².

For additional guidance regarding dose, see Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla., United States of America; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; Speight et al. (1997) Avery's Drug Treatment: A Guide to the Properties, Choice. Therapeutic Use and Economic Value of Drugs in Disease Management, 4th ed. Adis International, Auckland/Philadelphia, United States of America; Duch et al. (1998) Toxicol Lett 100-101:255-263.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Cloning of Mouse and Human SLC26A6

Human SLC26A6 exons were initially identified in draft sequences of the BAC clone RP11-148G20 and the PAC clone RP4-751E10 by performing tBLASTn searches of the HTGS database with SLC26A1, SLC26A2, SLC26A3, and SLC26A4 protein sequences as queries.

A BLASTn search of mouse ESTs using the extracted exon contig yielded a Sugano mouse I.M.A.G.E. clone (Clone ID No. 2,076,921) having 5′ and 3′ EST entries that displayed modest homology to the amino- and carboxyl-termini of known SLC26 proteins. This full-length mouse SLC26A6 cDNA was obtained from Research Genetics, Inc. (Birmingham, Ala., United States of America) and was sequenced on both strands using fluorescent dye terminator chemistry (available from Applied Biosystems of Foster City, Calif., United States of America).

A pair of PCR primers (SEQ ID NOs:62-63) was designed using the mouse cDNA sequence and human genomic data. The primers were used to clone the open reading frame of human SLC26A6 from human kidney RNA (available from BD Biosciences Clontech of Palo Alto, Calif., United States of America).

LA TAQ™ DNA polymerase (TaKaRa of Verivers, Belgium) was used for amplification reactions with the following amplification protocol: 30 cycles of denaturation at 98° C. for 30 seconds followed by amplification/extension at 68° C. for 6 minutes. Amplified PCR products were subcloned in the pCR2.1 vector by the TA CLONING® method (Invitrogen Corporation of Carlsbad, Calif., United States of America). The 3′ UTR was characterized by sequencing a number of human 3′ EST clones, including I.M.A.G.E. Clone ID Nos: 2,621,351 and 447,726.

A BLASTn search of a mouse genomic database (Celera of Rockville, Md., United States of America) yielded a 500 kb contig containing the 11 kb mSLC26A6 gene. A subsequent BLASTn search of mouse ESTs was performed using a 1.7 kb region between the start of EST 2,076,921 and the 3′ UTR of the upstream gene, flamingo 1 (FMI-1)/multiple EGF-like repeats factor 2 (MEGF-2). Three 5′ ESTs were identified in the RIKEN database (RIKEN Genome Sciences Center, of Yokohama, Japan), and were determined to overlap with EST 2,076,921. This alternative 5′ end was cloned by RT-PCR from mouse intestinal total RNA, using a sense primer in exon 1a (SEQ ID NO:64) and an antisense primer in exon 4 (SEQ ID NO:65). The equivalent human isoforms were cloned by RT-PCR using an exon 1a sense primer (SEQ ID NO:66) and an exon 3 anti-sense primer (SEQ ID NO:67) and human kidney RNA as template.

Analysis of nucleotide and amino acid sequences was performed using VECTOR NTI® 6.0 software (InforMax, Inc. of Bethesda, Md., United States of America), GRAIL® software (Lockheed Martin Energy Research Corporation of Oak Ridge, Tenn., United States of America) (Roberts, 1991; Xu et al., 1994; Uberbacher et al., 1996) (available at http://compbio.ornl.gov/Grail-1.3), Phosphobase (Kreegipuu et al., 1999) (http:www.cbs.dtu.dk/databases/PhosphoBase/), TESS (Schug & Overton, 1997); available at http://www.cbil.upenn.edu/cgi-bin/tess), Matinspector (Quandt et al., 1995); available from Genomatix Software GmbH of Munich Germany and at http://transfac.qbf.de/cgi-bin/amtSearch/matsearch.pl), and Prosite (Bucher & Bairoch, 1994; Hofmann et al., 1999) (http://www.expasv.ch/prosite/). A comparison of mouse SLC26A6 and human SLC26A6 revealed 78% identity at the amino acid level.

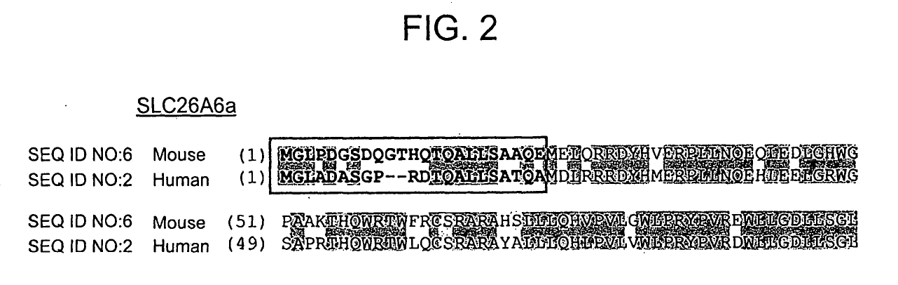

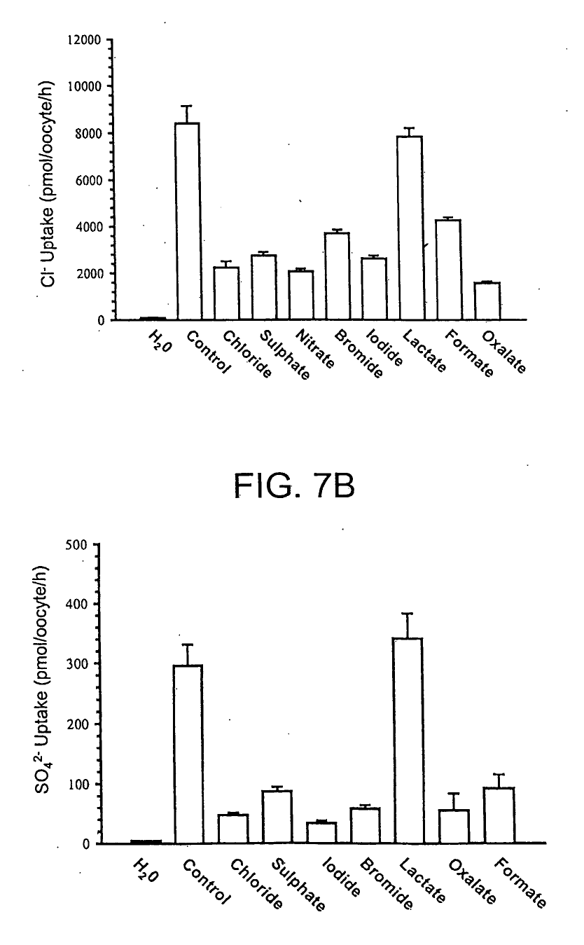

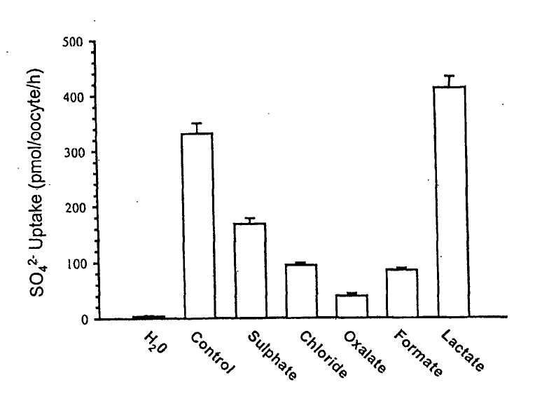

The inclusion of exon 1b (FIG. 2) in the longer SLC26A6b transcript results in a protein that is 21-23 amino acids shorter than SLC26A6a since this isoform uses a start codon in exon 2. The predicted start codons in exon 1a and exon 2 include reasonable Kozak sites (Kozak, 1996), with purines at position −3 and guanine at position +4. The amino acid sequence TQALLS (SEQ ID NO:68), which does not contain predicted phosphorylation sites, is conserved in the amino terminal region. Mouse SLC26A6b, which lacks the amino terminal extension is functional (FIGS. 7-10), and thus the amino terminal sequence is not required for transport activity.

### Example 2

### Genomic Localization of SLC26A6

The genomic organization of the chromosomal regions in which mouse SLC26A6 and human SLC26A6 reside is also conserved, such that both genes are flanked on the 5′ ends by the FMI-1/MEGF-2 gene and at the 3′ end by the UQCRC1 and ColA7 genes (Hoffman et al., 1993; Li et al., 1993).

Human STSs and previously localized genes were used to localize human SLC26A6 on chromosome 3p21 between markers D3S3582 and D3S1588. The mouse ColA7 gene is positioned about 40 kb 3′ to SLC26A6 within a 500 kb genomic contig, and thus is physically linked to SLC26A6. ColA7 is localized on mouse chromosome 9 at 61.0 cm (Li et al., 1993), and thus SLC26A6 is localized at ˜61 cM, which is a syntenic segment of human chromosome 3p21.

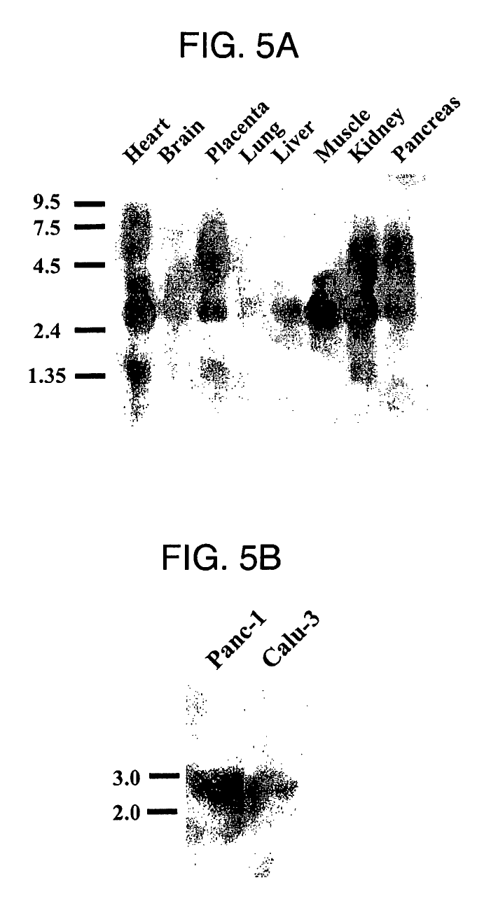

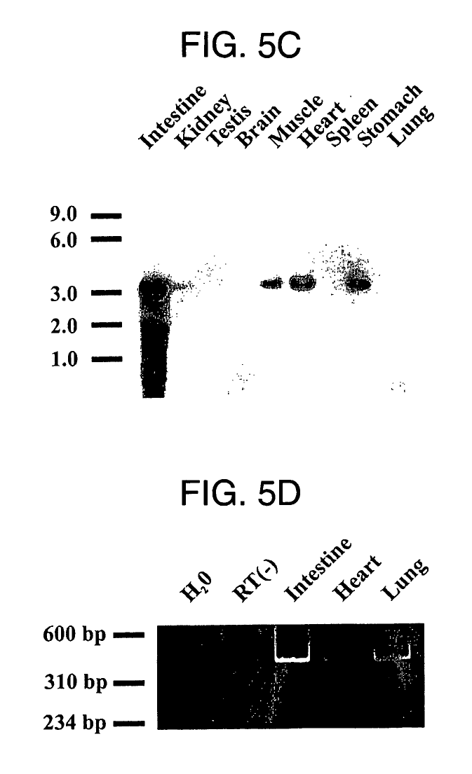

Mouse SLC26A6 and human SLC26A6 share a similar organization, encompassing 21 coding exons and ˜10 kb of genomic DNA. Intron-exon boundaries for mouse SLC26A6 are presented in Table 2 and are set forth as SEQ ID NOs:14-55. Both genes include an alternative 5′ non-coding exon (exon 1b). Non-quantitative RT-PCR (FIG. 5D) suggests that the isoform in which exon 1 b has been spliced out, denoted SLC26A6a, is expressed at a lower level than SLC26A6b.

### Example 3

### Northern Analysis of SLC26A6

RNA was extracted from C57BU6J mice and human cell lines using guanidine isothiocyanate and cesium chloride. The human pancreatic Panc-1 cell line and the human pulmonary Calu-3 cell line were obtained from the American Type Culture Collection (ATCC of Manassas, Va., United States of America). Calu-3 is a model for pulmonary submucosal gland serous epithelial cells (Lee et al., 1998), and Panc-1 is a model for pancreatic ductal epithelial cells (Elgavish & Meezan, 1992).

Total RNA (10 μg/lane) was size fractionated by electrophoresis (5% formaldehyde, 1% agarose), and transferred to a nylon membrane (Stratagene of La Jolla, Calif., United States of America). The blot was hybridized sequentially with ³²P-labeled randomly-primed probes corresponding to full-length GAPDH (glyceraldehyde-3-phosphate dehydrogenase) and a 3′ probe from mSLC26A6 (nucleotides 2239-2673 of mouse SLC26A6b, SEQ ID NO:7).

Northern blots prepared using 2 μg/lane of poly-A+ RNA were obtained from BD Biosciences Clontech of Palo Alto, Calif., United States of America. The blots were hybridized to a human SLC26A6a probe (nucleotides 2090-2587 of human SLC26A6a, SEQ ID NO:1) and a β-actin probe.

Hybridization of all blots was performed overnight at 42° C. in Express-Hyb solution (Clontech of Palo Alto, Calif., United States of America). Membranes were washed twice for 10 minutes at room temperature in 2×SSCP/0.1% SDS, and twice for 1 hour at 65° C. in 0.1×SSCP/0.1% SDS.

Northern blot analysis of human tissues indicated that human SLC26A6 is widely expressed. A 3.0 kb transcript was detected in kidney, muscle, pancreas, intestine, and heart (FIG. 5A). Minor bands of higher molecular weight (˜4 kb and ˜6 kb) were also detected in several tissues and are predicted to represent incompletely spliced transcripts. These extra bands are unlikely to result from cross-hybridization to other family members, since a non-coding probed was used.

Human SLC26A6 is also robustly expressed in the human Calu-3 and Panc-1 cell lines (FIG. 5B). Mouse SLC26A6 is also broadly expressed (FIG. 5C).

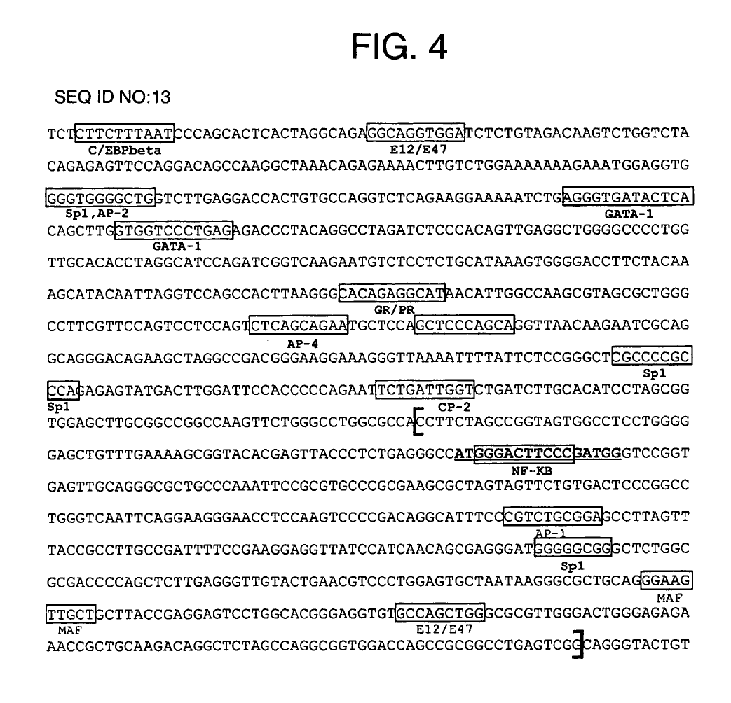

The widespread expression of SLC26A6 is consistent with the presence of a CpG island overlapping exon 1a (FIG. 4), which is conserved in both human SLC26A6 and mouse SLC26A6. The most 5′ mouse SLC26A6a ESTs begin ˜100 bp 5′ of the start codon in exon 1a, and thus the transcriptional start site lies at or 5′ to the most 5′ site of exon 1a. The genomic DNA flanking mouse SLC26A6 exon 1a suggests that SLC26A6 uses a TATA-less promoter that is rich in Sp1 binding sites (FIG. 4).

### Example 4

### Cloning of SLC26A1

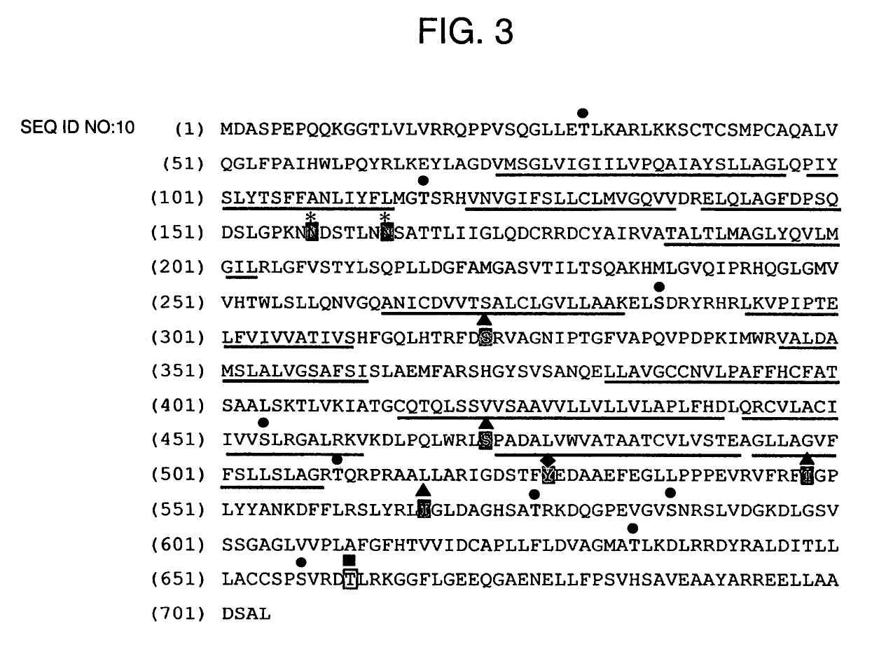

A full-length mouse SLC26A1 cDNA was identified on an EST cDNA (I.M.A.G.E. Clone ID No. 1,450,460). Mouse SLC26A1 protein (FIG. 3) is 91% identical to rat SLC26A1 protein, and 76% identical to human SLC26A1 protein. Large genomic contigs containing the mouse and human SLC26A 1 genes reveal a conserved organization, such that they are both flanked at the 5′ end by the FGFRL-1 (Wiedemann & Trueb, 2001), GAK (Kimura et al., 1997), and DAGK4 (Endele et al., 1996) genes and at the 3′ end by the L-iduronidase gene. Mouse FGFRL-1 and L-iduronidase have both been localized on mouse chromosome 5 at ˜57 cM (Wiedemann & Trueb, 2001), syntenic with the region of human chromosome 4p16 containing SLC26A1, GAK (Kimura et al., 1997), and DAGK4 (Endele et al., 1996).

The genomic organization of the human and mouse SLC26A1 genes is also conserved, although analysis of a number of 5′ mouse SLC26A1 ESTs reveals the existence of two 5′ non-coding exons in the mouse gene. The 5′ non-coding exon positioned more 3′ relative to the alternate 5′ exon was denoted exon 1b. Exon 1b is excluded from a number of ESTs indicating that it is alternatively spliced. The relative position of the junction between the two coding exons of SLC26A 1 and SLC26A2 (DTST), which together form a separate branch of the gene family, is conserved in the respective mouse and human genes.

### Example 5

### Expression of SLC26A6 and SLC26A 1 in Xenopus laevis Oocytes

Full-length mouse SLC26A6b and SLC26A 1 cDNAs were cloned into the Xenopus expression vector pGEMHE (Liman et al., 1992). The SLC26A6 and SLC26A1 expression constructs were linearized, and cRNA was transcribed in vitro using T7 RNA polymerase and a MMESSAGE MMACHINE® transcription kit (Ambion, Inc. of Austin, Tex., United States of America). Defolliculated oocytes were injected with 25 nl to 50 nl of water or with a solution containing cRNA at a concentration of 0.5 μg/μl (12.5 ng to 25 ng per oocyte) using a Nanoliter-2000 injector (WPI Instruments of Sarasota, Fla., United States of America). Oocytes were incubated at 17° C. in 50% Leibovitz's L-15 media supplemented with penicillin/streptomycin (1000 units/ml) and glutamine for 2-3 days for uptake assays.

### Example 6

### Anion Transport

For sulphate uptake assays, oocytes were pre-incubated for 20 minutes in chloride-free uptake medium (100 mM NMDG gluconate, 2 mM potassium gluconate, 1 mM calcium gluconate, 1 mM magnesium gluconate, 10 mM HEPES-Tris, pH 6.0 or pH 7.5 as indicated), followed by a 60-minute period for uptake in the same medium supplemented with 1 mM K₂³⁵SO₄ (40 μCi/ml). The cells were then washed three times in uptake buffer with 5 mM cold K₂SO₄ to remove tracer activity in the extracellular fluid. The oocytes were dissolved individually in 10% SDS, and tracer activity was determined by scintillation counting. Uptake of chloride, formate, and oxalate was assayed using the same chloride-free uptake solutions, substituting 8.3 mM ³⁶Cl, 500 μM [¹⁴C]oxalate, or 50 μM [¹⁴C]formate for labeled sulphate.

For sulphate exchange and cis-inhibition experiments, the concentration of NMDG-gluconate in the uptake solution was adjusted to maintain isotonic osmolality, which was confirmed experimentally using a FISKE® osmometer (Fiske Associates, Inc. of Bethel, Conn., United States of America).

### SLC26A6 Anion Transport

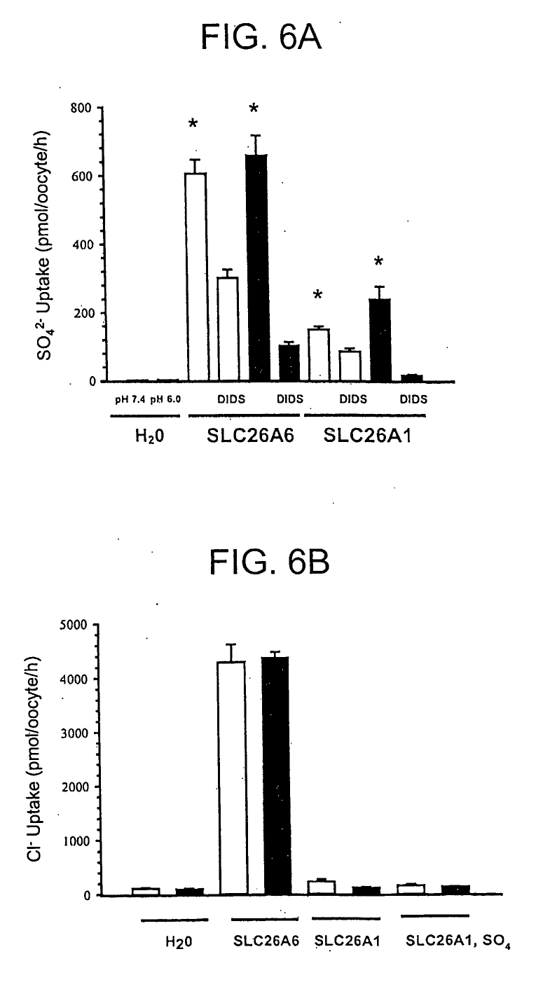

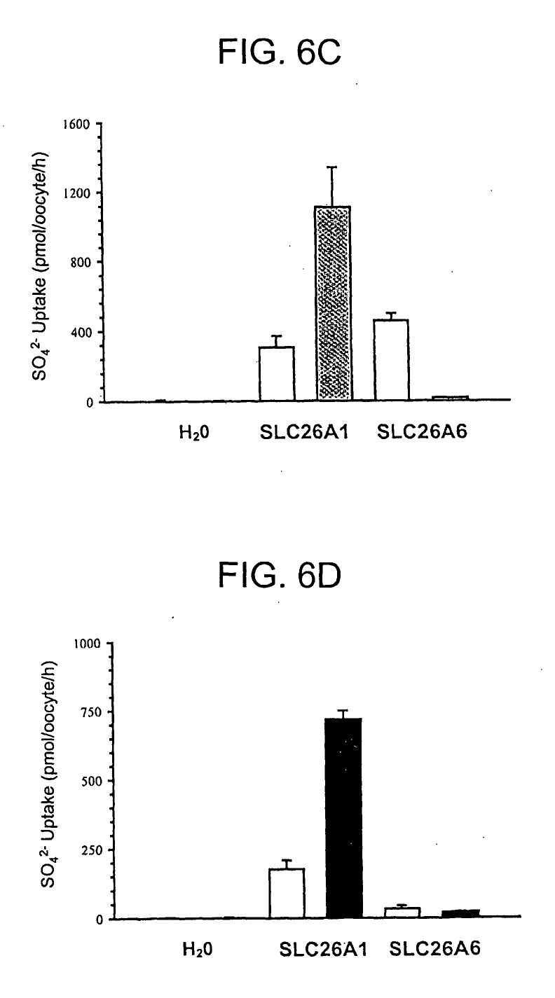

As shown in FIG. 6A, SLC26A6b transported sulphate independent of Cl⁻ (602±0 pmol/oocyte/hour at pH 7.4 versus 2.0±0.2 pmol/oocyte/hour in water-injected controls). SLC26A6b also transported sulphate independent of Na⁺ (652±57 pmol/oocyte/hour versus 5.8±0.9 pmol/oocyte/hour in water-injected controls) (FIG. 6A). Sulphate uptake was not significantly altered at pH 7.4 versus pH 6.0, although SLC26A6b was more sensitive to DIDS at pH 6.0 (101±10 pmol/oocyte/hour) than at pH 7.4 (301±24 pmol/oocyte/hour).

SLC26A6b-injected oocytes also retained ³⁶Cl⁻ (FIG. 6B). In this case, a consistent difference between uptake at pH 7.4 (4345±243 pmol/oocyte/hour versus 10±12 pmol/oocyte/hour in water-injected controls) and uptake at pH 6.0 (4193±109 pmol/oocyte/hour versus 99±16 pmol/oocyte/hour in water-injected controls) was not observed.

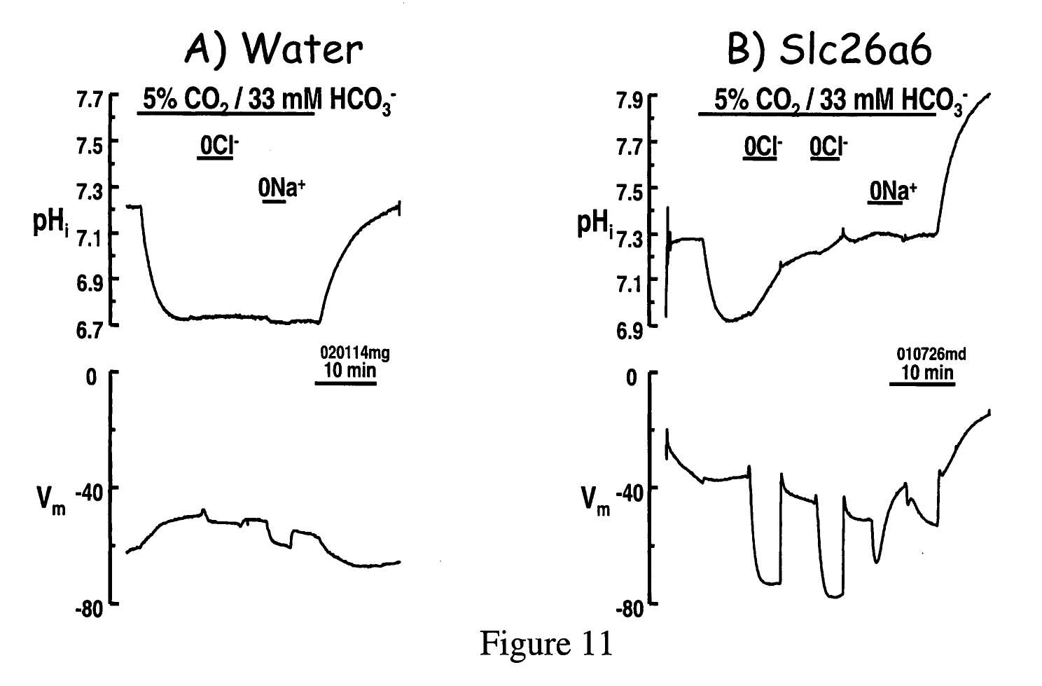

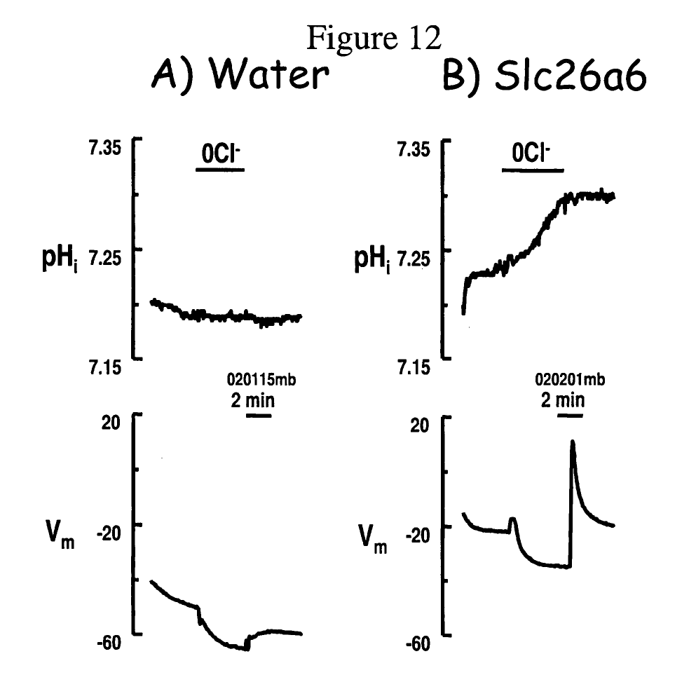

Since the concentrations of SO₄²⁻ and Cl⁻ in Xenopus oocytes are about 1 mM (Chernova et al., 1997) and about 30 mM (Romero et al., 2000), respectively, a significant component of the measured uptakes represented SO₄²⁻—SO₄²⁻ and Cl⁻—Cl⁻ exchange at the concentrations used in the extracellular uptake medium (1 mM for SO₄²⁻ and 8 mM for Cl⁻). SLC26A6b also mediated Cl⁻—HCO₃⁻ exchange (FIG. 11), and thus, the observed lack of stimulation by more acidic extracellular medium (FIG. 6B) was surprising.

A shared property of the SLC26A1, SLC26A2, SLC26A3, and SLC26A4 exchangers is cis-inhibition by transported substrates (Satoh et al., 1998; Moseley et al., 1999; Scott & Karniski, 2000; Knauf et al., 2001). To assess the repertoire of substrates exchanged with Cl⁻, SLC26A6b injected oocytes were incubated in the presence of sulphate, formate, halides, nitrate, and lactate. Among this group, sulphate, formate, halides, and nitrate, but not lactate significantly inhibited Cl⁻—Cl⁻ exchange (FIG. 7A). A similar profile was obtained for SO₄²⁻ transport (FIG. 7B).

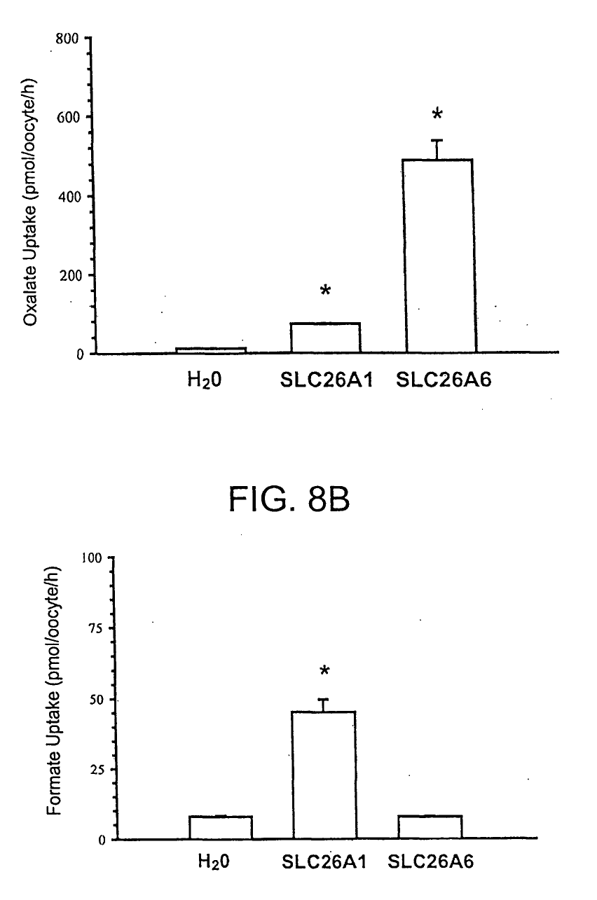

The observed profile of cis-inhibition was similar to that of the renal Cl⁻-formate exchanger (Karniski & Aronson, 1987). As predicted, SLC26A6b-injected oocytes transported both oxalate (487±50 pmol/oocyte/hour vs. 12±1 pmol/oocyte/hour in water-injected controls, FIG. 8A) and formate (45±5 pmol/oocyte/hour vs. 7 pmol/oocyte/hour in water-injected controls, FIG. 8B).

Sulphate exchange was also measured in SLC26A6b-injected oocytes in the presence of extracellular substrates (Scott & Karniski, 2000), and cis-inhibition was measured in SLC26A6b-injected oocytes in the presence of sulphate. Since SLC26A4 is known to transport formate and Cl⁻ but neither oxalate (Scott & Karniski, 2000) nor SO₄²⁻ (Scott et al., 1999), it was proposed that SLC26A6b does not catalyze formate-SO₄²⁻ exchange. Surprisingly, SLC26A6b clearly mediated exchange of SO₄²⁻ with SO₄²⁻, Cl⁻, formate and oxalate (FIG. 7C).

### SLC26A1 Anion Transport

SLC26A1 mediated SO₄²⁻ and oxalate uptake (72±2 pmol/oocyte/h, FIGS. 6A and 8A), but not formate (7 pmol/oocyte/h). The absolute transport rates were significantly lower for both SO₄²⁻ and oxalate when compared to SLC26A6b-facilitated transport. However, SO₄²⁻ transport rates for SLC26A1 were much higher in the presence of extracellular Cl⁻, closer to the values of SLC26A6 in the absence of Cl⁻.

SLC26A1 mediated minimal Cl⁻—Cl⁻ exchange in either the presence or absence of 25 mM SO₄²⁻ (FIG. 6D), suggesting that Cl⁻ and SO₄²⁻ are not synergistically co-transported by SLC26A1. The lack of Cl⁻—Cl⁻ exchange is consistent with transport studies using basolateral membrane vesicles from renal cortex, which indicate that the basolateral SO₄²⁻—HCO₃⁻ exchanger does not transport Cl⁻ (Kuo & Aronson, 1988).

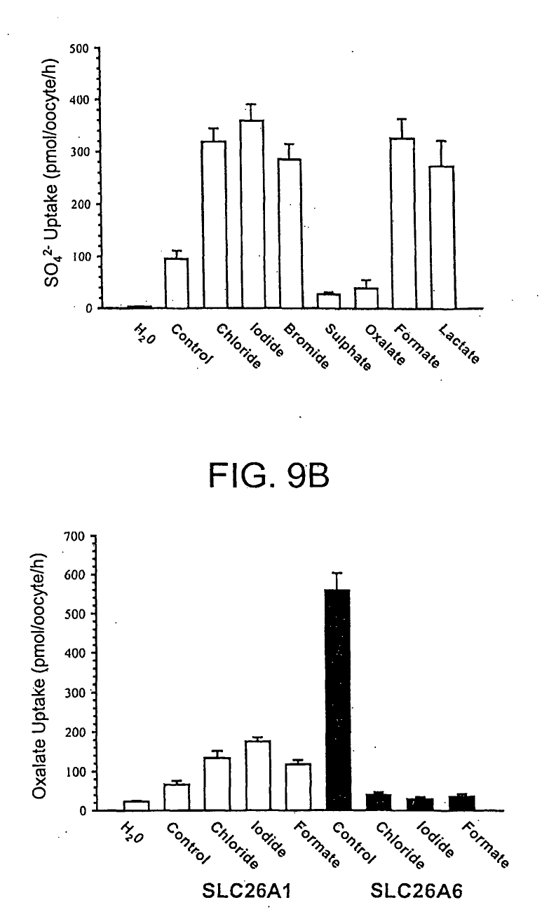

Several monovalent ions (halides, formate, and lactate) were observed to activate SO₄²⁻ transport via SLC26A1, although Cl⁻ (FIG. 6B) and formate (FIG. 8B) were not transported. In contrast, the divalent substrates SO₄²⁻ and oxalate were strongly cis-inhibitory to SO₄²⁻ transport via SLC26A1 (FIG. 9A).

Both I⁻ and Br⁻ were observed to be cis-inhibitory to SO₄²⁻ transport and to Cl⁻ transport by SLC26A6, indicating that they are potential substrates of this transporter. The activation of SLC26A1 was not unique to SO₄²⁻ transport, since oxalate transport in SLC26A 1-injected oocytes is also higher in the presence of monovalent anions (FIG. 9B). In this experiment, SLC26A6 served as a control because oxalate transport by oocytes injected with this cRNA was strongly cis-inhibited by these anions (FIG. 9B).

### SLC26A2 Anion Transport

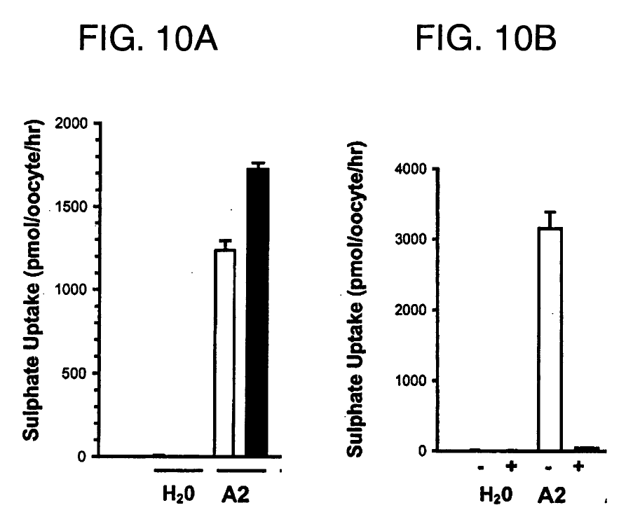

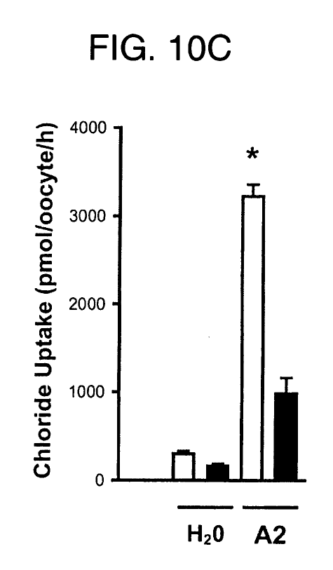

Oocytes expressing mouse SLC26A2 mediated robust ³⁵SO₄²⁻ uptake which is increased significantly at pH 6.0 (FIG. 10A). The addition of extracellular Cl⁻ significantly inhibited ³⁵SO₄²⁻ uptake by SLC26A2 (FIG. 10B). SLC26A2-injected oocytes also mediated significant ³⁶Cl⁻, uptake (FIG. 10C), an observation that has not been previously made for this anion exchanger. Since the concentration of Cl⁻ in Xenopus oocyte cytoplasm is ˜30 mM (Romero, 2000), versus 8 mM in the extracellular uptake medium, a significant component of the uptake activity probably represents Cl⁻—Cl⁻ exchange.

### Example 7

### Electrogenic Transport

For electrophysiological measurements, oocytes were studied 3 days to 11 days following injection of SLC26 expression constructs. CO₂/HCO₃-free ND96 medium contained 96 mM NaCl, 2 mM KCl, 1 mM MgCl2, 1.8 mM CaCl₂, and 5 mM HEPES (pH 7.5 and 195-200 mOsm). For CO₂/HCO₃-equilibrated solutions, 33 mM NaHCO₃ replaced 33 mM NaCl. In “O—Na⁺” solutions, choline replaced Na⁺. In “O—Cl⁻” solutions, gluconate replaced Cl. All solutions were titrated to pH 7.5, and were continuously bubbled with CO₂-balanced O₂ to maintain pCO₂ and pH. Ion selective microelectrodes were prepared, calibrated, and employed as described by Romero et al. (1998) Am J Physiol 274:F425-432 and by Romero et al. (2000) J Biol Chem 275:24552-24559. All pH electrodes had slopes of at least −56 mwdecade change.

To determine whether SLC26A6 functions as a Cl⁻—HCO₃⁻ exchanger, intracellular pH (pHᵢ) was measured in response to the manipulation of bath HCO₃⁻ and Cl⁻. The initial addition of CO₂/HCO₃⁻ to the bath solution resulted in the acidification of oocytes due to CO₂ plasma membrane diffusion, then intracellular hydration and dissociation forming intracellular H⁺ and HCO₃⁻ ions.

FIG. 11A shows that a water-injected oocyte exposed to 5% CO₂/33 mM HCO₃⁻ (pH 7.5) acidified by 0.44 pH units (−0.46±0.01, n=8) at an initial rate of 46×10⁻⁴ pH units/sec (460×10⁻⁵ pH units/sec; −382±19×10⁻⁵ pH units/sec, n=8). The initial intracellular pH (pHᵢ) of SLC26A6-injected oocytes was essentially the same as that of water controls (water, 7.26±0.03, n=8; SLC26A6, 7.29±0.03, n=10). Addition of 5% CO₂/33 mM HCO₃⁻ produced a fall in pHᵢ of 0.50 pH units (−0.46±0.02, n=10) at an initial rate of 35×10⁻⁴ pH units/sec (350×10⁻⁵ pH units/sec; −387±15×10⁻⁵ pH units/sec, n=10). SLC26A6-injected oocytes were depolarized (−26.3±4.5 mV, n=10) compared to control oocytes (−44.8±4.3 mV, n=8). The addition of HCO₃⁻ produced a slight but abrupt depolarization in SLC26A6-injected oocytes (3.1±0.6 mV, n=9). Cl⁻ replacement (gluconate) did not affect pHᵢ of the water control (+6.0±2.2×10⁻⁵ pH units/sec, n=8; FIG. 11A). However, Cl⁻ removal increased pHᵢ in SLC26A6-injected oocytes at the rate of 44×10⁻⁵ pH units/sec (+72±8.8×10⁻⁵ pH units/sec, n=10; FIG. 11B), which ceased after Cl⁻ re-addition. A plot of pHᵢ and and a plot of Vₘ for an individual SLC26A6-injected oocyte are shown in FIG. 11B. Surprisingly, gluconate replacement evoked a 37 mV hyperpolarization (−22.7±2.9 mV, n=9; vs.+0.2±2.0 mV, n=8 for controls). A second Cl⁻ removal increased the alkalinization rate to 28×10⁻⁵ pH units/sec (+41±6.2×10⁻⁵ pH units/sec, n=8; FIG. 11B) and reproduced the hyperpolarization (−18.6±3.8 mV, n=8).

FIGS. 11A and 11B illustrate an experiment with individual water-injected and SLC26A6-injected oocytes. These observations have been repeated using SLC26A6-injected oocytes from five separate frogs. In all the experiments using SLC26A6-injected oocytes, the second alkalinization induced by Cl⁻ removal, which occurs at a higher pHI, has a lower rate (+72×10⁻⁵ pH units/sec for the first alkalinization, and +41×10⁻⁵ pH units/sec, versus +6.0×10⁻⁵ pH units/sec for the single Cl⁻ removal in water-injected oocytes).

In another set of experiments, Na⁺ was replaced with choline to test cation dependence of SLC26A6. Na⁺ removal and replacement did not obviously affect pHᵢ. Prior to CO₂ removal, pHᵢ rose to 7.2, which is approximately the non-HCO₃⁻ level. Removal of 5% CO₂/33 mM HCO₃⁻ elicited a robust alkalinization and pHᵢ overshoot to 7.9 (7.83±0.07, n=10), indicative of cellular HCO₃⁻ loading (ΔpHᵢ for SLC26A6-injected oocytes was +0.53±0.07, n=10). This overshoot was not observed in control oocytes (ΔpHᵢ for controls was +0.02±0.04, n=8).

Since mouse SLC26A6 clearly functions as a Cl⁻—HCO₃⁻ exchanger, Cl⁻—OH⁻ exchange via SLC26A6 was also tested. For these experiments, non-HCO₃⁻ solutions were continuously bubbled with 100% O₂. FIGS. 12A and 12B illustrate the non-HCO₃⁻ responses of control and SLC26A6-injected oocytes. Removal of bath Cl⁻ from control oocytes (FIG. 12A) did not change pHᵢ (−2.1±1.8×10⁻⁵ pH units/sec, n=7) or Vₘ (−0.6±3.5 mV, n=7). In contrast, removal of bath Cl⁻ alkalinized (+27±6.4×10⁻⁵ pH units/sec, n=6) and hyperpolarized (−7.3±2.8 mV, n=8) SLC26A6-injected oocytes (FIG. 12B). Cl⁻ re-addition to the bath stopped the alkalinization and returned Vₘ to the initial value.

### Example 8

### Identification of SLC26 Orthologs from Other Species

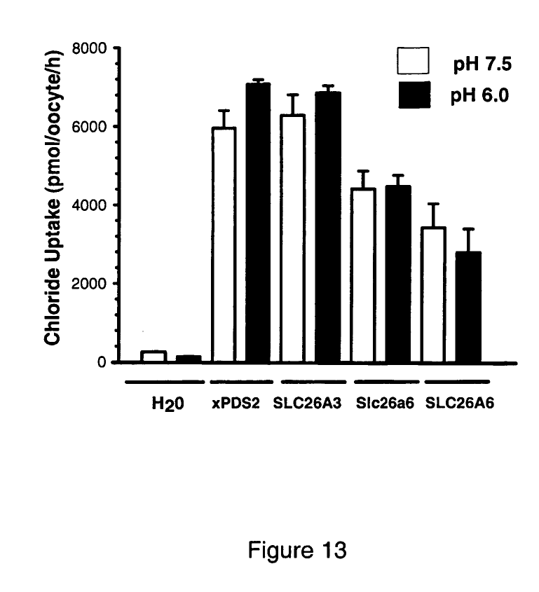

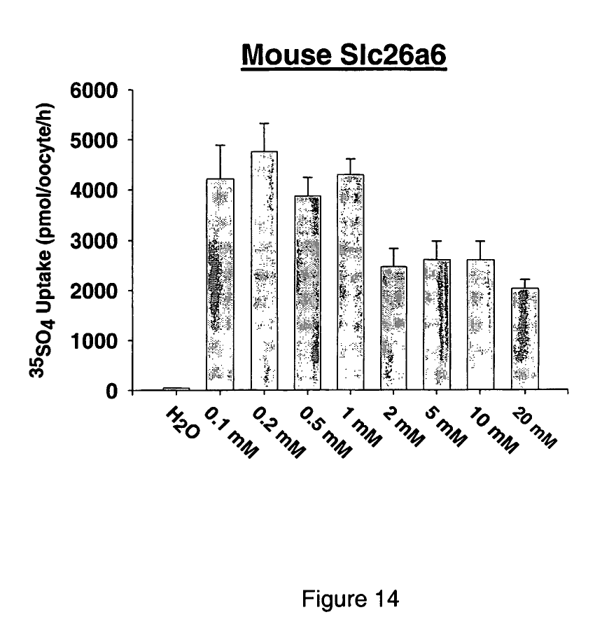

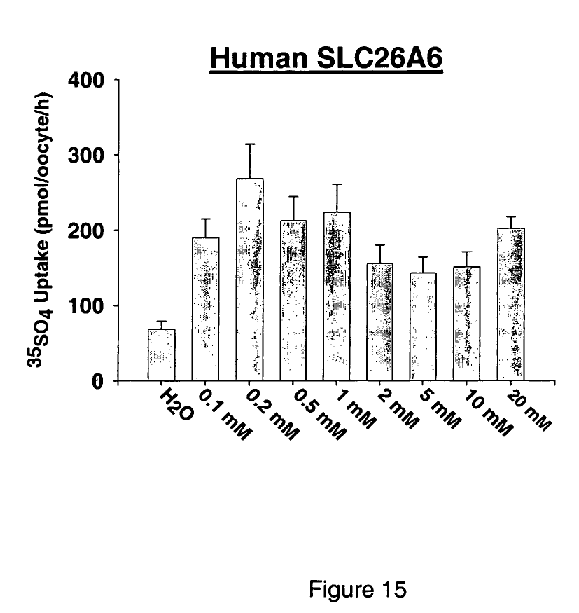

A feature of the mammalian SLC26 gene family is relatively low conservation between orthologs in mouse and man; the percent amino acid identity ranges from a low of 76% (SLC26A8) to a high of 90% (SLC26A9), versus the reported median of 86% for mouse and human orthologous genes. Makalowski, W. & Boguski, 1998. This sequence divergence is reflected in functional variation, which can be exploited for structure-function analysis. Human SLC26A6 and murine Slc26a6 appear to differ in their functional characteristics. Although SLC26A6 clearly transports Cl⁻ (FIG. 13), absolute rates of SO₄²⁻ transported by this exchanger are much lower (˜200 pmol/oocyte/hr vs. ˜4,000 pmol/oocyte/hr) than those measured for Slc26a6 (FIGS. 14 and 15, showing SO₄²⁻ uptakes at increasing concentrations of extracellular SO₄²⁻ with stable amount of radioactive ³⁵SO₄²⁻).

While orthologous pairs of murine and human SLC26 exchangers are of use for structure-function analysis, full-length cDNAs from several other species were also identified. To clone the pig SLC26A6 ortholog, overlapping pig SLC26A6 ESTs in Genbank were used to construct the entire cDNA in silico; this cDNA was then cloned by long-range RT-PCR from the LLC-PK1 cell line, a porcine cell-culture model of the renal proximal tubule. The pig SLC26A6a protein is 77% identical to murine Slc26a6 and 80% identical to human SLC26A6. SEQ ID NOs:90-91 are the nucleic acid and amino acid sequences, respectively, of SLC26A6a isolated from pig (Sus scrofa).

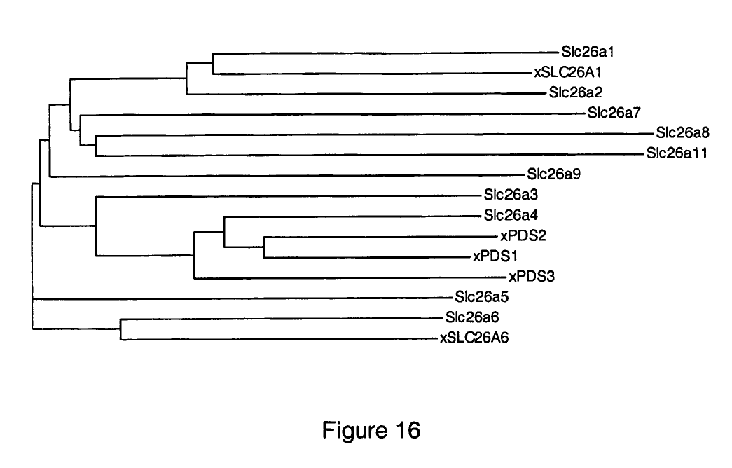

Xenopus laevis EST cDNAs derived from the orthologs of several SLC26 genes were also identified. This effort has resulted in the identification of the Xenopus SLC26a6 ortholog (“xSLC26A6”), SLC26A1 ortholog (“xSLC26A1”), and three apparent orthologs of SLC26A4 (pendrin or PDS), provisionally denoted “xPDS1-3”. A phylogenetic tree shows these relationships well (FIG. 16). SEQ ID NOs: 80-85 are the nucleic acid (even SEQ ID NOs:80, 82, and 84) and amino acid sequences (odd SEQ ID NOs: 81, 83, and 85) of SLC26A4 (PDS1-3). SEQ ID NOs: 86-87 are the nucleic acid and amino acid sequences, respectively, of SLC26A1 isolated from Xenopus laevis. SEQ ID NOs: 88-89 are the nucleic acid and amino acid sequences, respectively, of SLC26A6 isolated from Xenopus laevis.

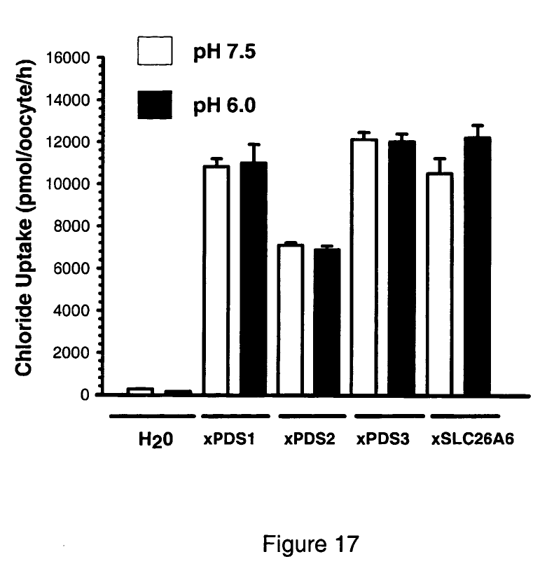

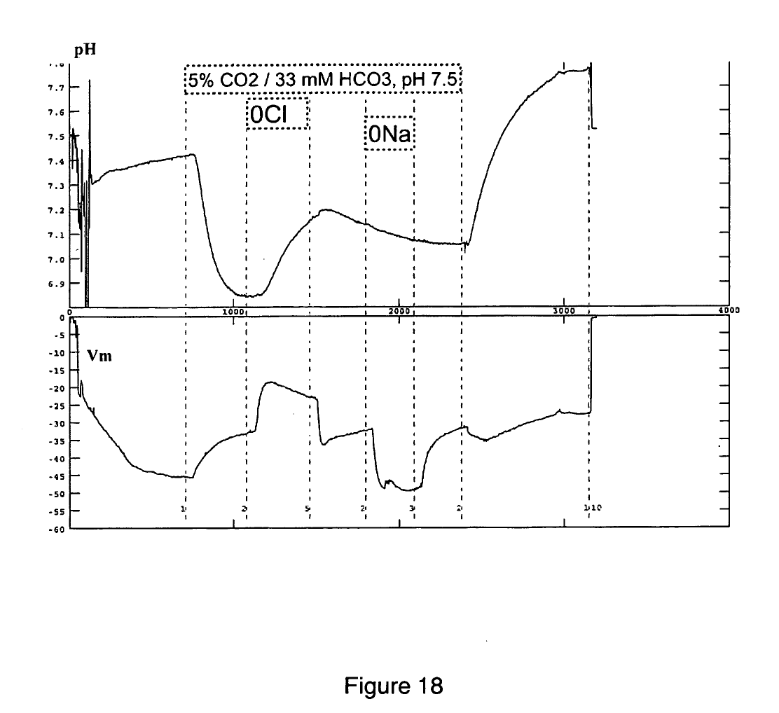

One or more of these xPDS genes likely corresponds to the Xenopus descendant of mammalian SLC26A3. However, the xPDS proteins are much more homologous to mammalian SLC26A4/pendrin than to SLC26A3; xPDS1, xPDS2, and xPDS3 are respectively 67%, 59%, and 54% identical to mouse Slc26a4, versus 45%, 42%, and 40% identity to mouse Slc26a3. The xSLC26A6 exchanger and the three xPDS exchangers are capable of robust Cl⁻ transport when expressed in Xenopus oocytes (see FIG. 17). The xPDS2 protein can also clearly mediate Cl⁻—HCO₃⁻ exchange (see FIG. 18). As reported for human PDS/SLC26A6 (Scott, D. A., et al., 1999), none of the xPDS clones transport SO₄²⁻; these cDNAs will therefore be useful tools to identify the molecular determinants of monovalent specificity (i.e. domains that impart specificity for monovalent ions) in mammalian SLC26A4 and SLC26A3.

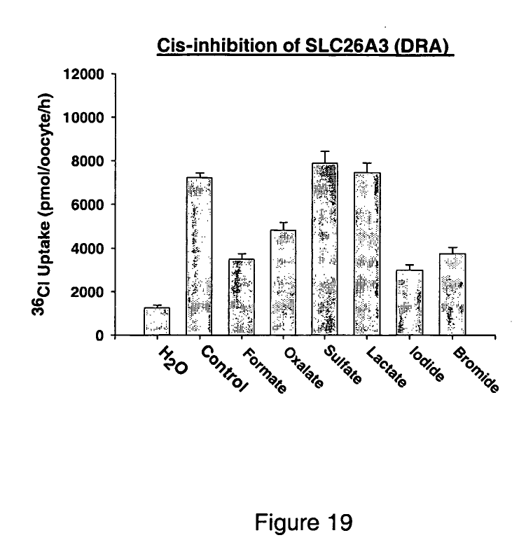

The SLC26A6 exchanger is a likely mediator of intestinal oxalate absorption and thus a potential therapeutic target in calcium-oxalate stone disease. Intestinal expression of the SLC26A3 or DRA protein is also robust, in keeping with the genetic role of SLC26A3 in congenital chloride-losing diarrhea. Hoglund, P. et al., 1996. SLC26A3 has reported to possess mediate oxalate transport; however, the data is not convincing (Moseley, R. H. et al., 1999; Silberg, D. G., et al., 1995), and it is difficult to understand why the SLC26A3 protein would transport oxalate when its close homolog SLC26A4 does not. Scott, D. A., et al., 1999; Scott, D.A. & Karniski, L. P., 2000. Cis-inhibition of the robust Cl transport mediated by SLC26A3-injected oocytes suggests that this protein transports minimal if any oxalate (FIG. 19). This suggests furthermore that SLC26A6 is the more important apical pathway for intestinal oxalate absorption.

### Example 9

### Generation of Ortholog-Specific SLC26 Antibodies

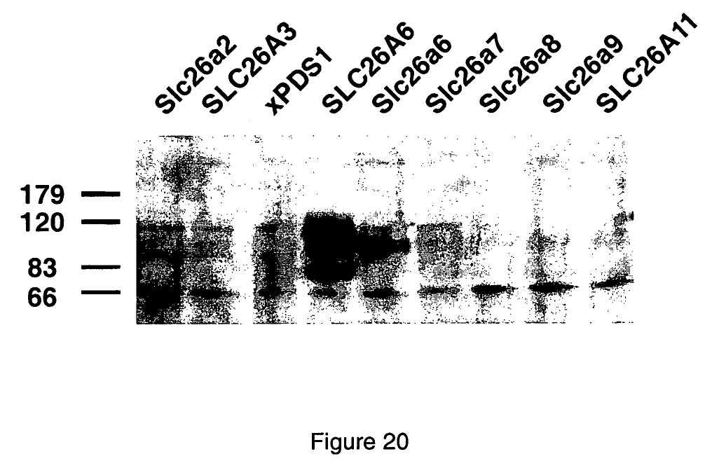

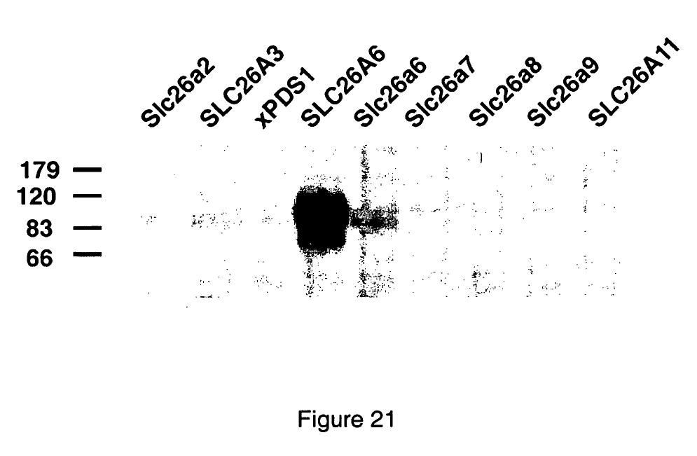

To facilitate the understanding of the physiological role of the SLC26A6/Slc26a6 anion exchanger a number of polyclonal antibodies were generated against unique epitopes within the murine Slc26a6 sequence. These include both an N-terminal antibody, directed against the sequence QEQLEDLGHWGPAAKTH (residues 40-56 of the Slc26a6a protein —SEQ ID NO:92) and a C-terminal antibody directed against the sequence KVHQGEELQDVVSSNQEDA (residues 631-649—SEQ ID NO:93). Both the N- and C-terminal antibodies recognize human SLC26A6 and murine Slc26a6 proteins, specifically a “core” (likely high-mannose) protein of −83 kDa and a complex glycoprotein of ˜110 kDa (FIGS. 20 and 21). High-titre antisera have also been obtained for Slc26a1/SLC26A1-specific and Slc26a2/SLC26A2-specific antigens, YRLTGLDAGHSATRKDQ (residues 564-580 of Slc26a1—SEQ ID NO:94) and KEQHNVSPRDSAEGNDS (residues 6-22 of SLC26A2—SEQ ID NO:95).

### REFERENCES

The references listed below as well as all references cited in the specification are incorporated herein by reference to the extent that they supplement, explain, provide a background for or teach methodology, techniques and/or compositions employed herein.
(1997) Current Protocols in Human Genetics on CD-Rom. John Wiley & Sons, New York.
Akiba Y, Furukawa O, Guth P H, Engel E, Nastaskin I, Sassani P, Dukkipatis R, Pushkin A, Kurtz I & Kaunitz J D (2001) Cellular Bicarbonate Protects Rat Duodenal Mucosa from Acid-Induced Injury. J Clin Invest 108:1807-1816.
Alam J & Cook J L (1990) Reporter Genes: Application to the Study of Mammalian Gene Transcription. Anal Biochem 188:245-254.
Altschul S F, Gish W, Miller W, Myers E W & Lipman D J (1990) Basic Local Alignment Search Tool. J Mol Biol 215:403-410.
Aravind L & Koonin E V (2000) The STAS Domain—a Link between Anion Transporters and Antisigma-Factor Antagonists. Curr Biol 10:R53-55.
Ausubel F, ed (1995) Short Protocols in Molecular Biology, 3rd ed. Wiley, New York.
Baggio B, Gambaro G, Marchini F, Cicerello E, Tenconi R, Clementi M & Borsatti A (1986) An Inheritable Anomaly of Red-Cell Oxalate Transport in “Primary” Calcium Nephrolithiasis Correctable with Diuretics. N Engl J Med 314:599-604.
Baher A G, Andres M L, Folz-Holbeck J, Cao J D & Gridley D S (1999) A Model Using Radiation and Plasmid-Mediated Tumor Necrosis Factor-Alpha Gene Therapy for Treatment of Glioblastomas. Anticancer Res 19:2917-2924.
Barton G J (1998) Protein Sequence Alignment Techniques. Acta Crystallogr D Biol Crystallogr 54:1139-1146.
Batzer M A, Carlton J E & Deininger P L (1991) Enhanced Evolutionary PCR Using Oligonucleotides with Inosine at the 3′-Terminus. Nucleic Acids Res 19:5081.
Bauminger S & Wilchek M (1980) The Use of Carbodiimides in the Preparation of Immunizing Conjugates. Methods Enzymol 70:151-159.
Beck L & Silve C (2001) Molecular Aspects of Renal Tubular Handling and Regulation of Inorganic Sulfate. Kidney Int 59:835-845.
Berkow R, Beers M H & Fletcher A J (1997) The Merck Manual of Medical Information, Home ed. Merck Research Laboratories, Whitehouse Station, N.J., United States of America.
Betageri G V, Jenkins S A & Parsons D L (1993) Liposome Drua Delivery Systems. Technomic Pub., Lancaster, Pa., United States of America.
Bissig M, Hagenbuch B, Stieger B, Koller T & Meier P J (1994) Functional Expression Cloning of the Canalicular Sulfate Transport System of Rat Hepatocytes. J Biol Chem 269:3017-3021.
Biwersi J & Verkman A S (1991) Cell-Permeable Fluorescent Indicator for Cytosolic Chloride. Biochemistry 30:7879-7883.
Biwersi J, Tulk B & Verkman A S (1994) Long-Wavelength Chloride-Sensitive Fluorescent Indicators. Anal Biochem 219:139-143.
Bodanszky M (1993) Principles of Peptide Synthesis, 2nd rev. ed. Springer-Verlag, Berlin/N.Y.
Brookes A J (1999) The Essence of SNPs. Gene 234:177-186.
Bucher P & Bairoch A (1994) A Generalized Profile Syntax for Biomolecular Sequence Motifs and Its Function in Automatic Sequence Interpretation. Proc Int Conf Intell Syst Mol Biol 2:53-61.
Capecchi M R (1989a) Altering the Genome by Homologous Recombination. Science 244:1288-1292.
Capecchi M R (1989b) Altering the Genome by Homologous Recombination. Science 244:1288-1292.
Chan D W (1996) Immunoassay Automation: An Updated Guide to Systems. Academic Press, San Diego.
Chernova M N, Jiang L, Crest M, Hand M, Vandorpe D H, Strange K & Alper S L (1997) Electrogenic Sulfate/Chloride Exchange in Xenopus Oocytes Mediated by Murine AE1 E699q. J Gen Physiol 109:345-360.
Chiang L W (1998) Detection of Gene Expression in Single Neurons by Patch-Clamp and Single-Cell Reverse Transcriptase Polymerase Chain Reaction. J Chromatogr A 806:209-218.
Choi J Y, Muallem D, Kiselyov K, Lee M G, Thomas P J & Muallem S (2001) Aberrant CFTR-Dependent HCO₃⁻ Transport in Mutations Associated with Cystic Fibrosis. Nature 410:94-97.
Conner B J, Reyes A A, Morin C, ltakura K, Teplitz R L & Wallace R B (1983) Detection of Sickle Cell Beta S-Globin Allele by Hybridization with Synthetic Oligonucleotides. Proc Natl Acad Sci USA 80:278-282.
Coughtrie M W, Sharp S, Maxwell K & Innes N P (1998) Biology and Function of the Reversible Sulfation Pathway Catalysed by Human Sulfotransferases and Sulfatases. Chem Biol Interact 109:3-27.
Cubitt A B, Heim R, Adams S R, Boyd A E, Gross L A & Tsien R Y (1995) Understanding, Improving and Using Green Fluorescent Proteins. Trends Biochem Sci 20:448-455.
Davis B A, Hogan E M & Boron W F (1992) Activation of Na—H Exchange by Intracellular Lithium in Barnacle Muscle Fibers. Am J Physiol 263:C246-256.
Deisenhofer J, Epp O, Miki K, Huber R & Michel H (1984) X-Ray Structure Analysis of a Membrane Protein Complex. Electron Density Map at 3 a Resolution and a Model of the Chromophores of the Photosynthetic Reaction Center from Rhodopseudomonas Viridis. J Moliol 180:385-398.
Delpire E, Lu J, England R, Dull C & Thorne T (1999) Deafness and Imbalance Associated with Inactivation of the Secretory Na—K-2Cl Co-Transporter. Nat Genet 22:192-195.
Duch D S, Rehberg B & Vysotskaya T N (1998) Volatile Anesthetics Significantly Suppress Central and Peripheral Mammalian Sodium Channels. Toxicol Lett 100-101:255-263.
Ebadi M S (1998) CRC Desk Reference of Clinical Pharmacology. CRC Press, Boca Raton, Fla., United States of America.
Elgavish A & Meezan E (1992) Altered Sulfate Transport Via Anion Exchange in CFPAC Is Corrected by Retrovirus-Mediated CFTR Gene Transfer. Am J Physiol 263:C176-186.
Endele S, Zabel B & Winterpacht A (1996) Assignment of the Human Diacylglycerol Kinase 4 (DagK4) Gene to Chromosome 4p16.3. Genomics 33:145-146.
European Patent No. 0 439 095
Everett L A & Green E D (1999) A Family of Mammalian Anion Transporters and Their Involvement in Human Genetic Diseases. Hum Mol Genet 8:1883-1891.
Falany C N (1997) Enzymology of Human Cytosolic Sulfotransferases. Faseb J 11:206-216.
Fewell J G, MacLaughlin F, Mehta V, Gondo M, Nicol F, Wilson E & Smith L C (2001) Gene Therapy for the Treatment of Hemophilia B Using PINC-Formulated Plasmid Delivered to Muscle with Electroporation. Mol Ther 3:574-583.
Flemstrom G & Isenberg J I (2001) Gastroduodenal Mucosal Alkaline Secretion and Mucosal Protection. News Physiol Sci 16:23-28.
Freireich E J, Gehan E A, Rall D P, Schmidt L H & Skipper H E (1966) Quantitative Comparison of Toxicity of Anticancer Agents in Mouse, Rat, Hamster, Dog, Monkey, and Man. Cancer Chemother Rep 50:219-244.
Freshney R1 (1987) Culture of Animal Cells: A Manual of Basic Technique, 2nd ed. A.R. Liss, New York.
Galietta L J, Springsteel M F, Eda M, Niedzinski E J, By K, Haddadin M J, Kurth M J, Nantz M H & Verkman A S (2001) Novel CFTR Chloride Channel Activators Identified by Screening of Combinatorial Libraries Based on Flavone and Benzoquinolizinium Lead Compounds. J Biol Chem 276:19723-19728.
Glover D M & Hames B D (1995) DNA Cloning: A Practical Approach, 2nd ed. IRL Press at Oxford University Press, Oxford/New York.
Goldman C K, Rogers B E, Douglas J T, Sosnowski B A, Ying W, Siegal G P, Baird A, Campain J A & Curiel D T (1997) Targeted Gene Delivery to Kaposi's Sarcoma Cells Via the FibroBLAST Growth Factor Receptor. Cancer Res 57:1447-1451.
Grassl S M (1996) Sulfate Transport in Human Placental Brush-Border Membrane Vesicles. Biochim Biophys Acta 1282:115-123.
Greeley T, Shumaker H, Wang Z, Schweinfest C W & Soleimani M (2001) Downregulated in Adenoma and Putative Anion Transporter Are Regulated by CFTR in Cultured Pancreatic Duct Cells. Am J Physiol Gastrointest Liver Physiol 281:G1301-1308.
Gregoriadis G (1993) Liposome Technology, 2nd ed. CRC Press, Boca Raton, Fla., United States of America.
Habib N A, Hodgson H J, Lemoine N & Pignatelli M (1999) A Phase I/II Study of Hepatic Artery Infusion with WTp53-CMV-Ad in Metastatic Malignant Liver Tumours. Hum Gene Ther 10:2019-2034.
Hagenbuch B, Stange G & Murer H (1985) Transport of Sulphate in Rat Jejunal and Rat Proximal Tubular Basolateral Membrane Vesicles. Pflugers Arch 405:202-208.
Haila S, Hastbacka J, Bohling T, Karjalainen-Lindsberg M L, Kere J & Saarialho-Kere U (2001) Slc26a2 (Diastrophic Dysplasia Sulfate Transporter) Is Expressed in Developing and Mature Cartilage but Also in Other Tissues and Cell Types. J Histochem Cytochem 49:973-982.
Haila S, Saarialho-Kere U, Karjalainen-Lindsberg M L, Lohi H, Airola K, Holmberg C, Hastbacka J, Kere J & Hoglund P (2000) The Congenital Chloride Diarrhea Gene Is Expressed in Seminal Vesicle, Sweat Gland, Inflammatory Colon Epithelium, and in Some Dysplastic Colon Cells. Histochem Cell Biol 113:279-286.
Harlow E & Lane D (1988) Antibodies: A Laboratory Manual. Cold Spring Harbor Laboratory Press, Cold Spring Harbor, N.Y.
Hastbacka J, de la Chapelle A, Mahtani M M, Clines G, Reeve-Daly M P, Daly M, Hamilton B A, Kusumi K, Trivedi B, Weaver A & et al. (1994) The Diastrophic Dysplasia Gene Encodes a Novel Sulfate Transporter: Positional Cloning by Fine-Structure Linkage Disequilibrium Mapping. Cell 78:1073-1087.
Henikoff J G, Pietrokovski S, McCallum CM & Henikoff S (2000) Blocks-Based Methods for Detecting Protein Homology. Electrophoresis 21:1700-1706.
Henikoff S & Henikoff J G (1992) Amino Acid Substitution Matrices from Protein Blocks. Proc Natl Acad Sci USA 89:10915-10919.
Henikoff S & Henikoff J G (2000) Amino Acid Substitution Matrices. Adv Protein Chem 54:73-97.
Hoffman G G, Lee S, Christiano A M, Chung-Honet L C, Cheng W, Katchman S, Uitto J & Greenspan D S (1993) Complete Coding Sequence, Intron/Exon Organization, and Chromosomal Location of the Gene for the Core I Protein of Human Ubiquinol-Cytochrome C Reductase. J Biol Chem 268:21113-21119.
Hofmann K, Bucher P, Falquet L & Bairoch A (1999) The Prosite Database, Its Status in 1999. Nucleic Acids Res 27:215-219.
Hoglund, P. et al. Mutations of the Down-regulated in adenoma (DRA) gene cause congenital chloride diarrhoea. Nat Genet 14:316-9. (1996).
Holmes R P, Goodman H O & Assimos D G (2001) Contribution of Dietary Oxalate to Urinary Oxalate Excretion. Kidney Int 59:270-276.
Homola J, Yee S & Gauglitz G (1999) Surface Plasmone Resnoance Sensors: Review. Sensors and Actuators B 54:3-15.
Huang C C, Novak W R, Babbitt P C, Jewett Al, Ferrin T E & Klein T E (2000) Integrated Tools for Structural and Sequence Alignment and Analysis. Pac Symp Biocomput:230-241.
Hutchens & Yip (1993) Rapid Communications in Mass Spectroscopy 7:576-580.
International Publication No. WO 97/47763
International Publication No. WO 98/10795
International Publication No. WO 99/26966
Ishikawa E (1999) Ultrasensitive and Rapid Enzyme Immunoassay. Elsevier, Amsterdam/N.Y.
Jayaraman S, Teitler L, Skalski B & Verkman A S (1999) Long-Wavelength Iodide-Sensitive Fluorescent Indicators for Measurement of Functional CFTR Expression in Cells. Am J Physiol 277:C1008-1018.
Jayaraman S, Haggie P, Wachter R M, Remington S J & Verkman A S (2000) Mechanism and Cellular Applications of a Green Fluorescent Protein-Based Halide Sensor. J Biol Chem 275:6047-6050.
Joyner A L (1993) Gene Targeting: A Practical Approach. Oxford University Press, Oxford/N.Y.
Karlin S & Altschul S F (1993) Applications and Statistics for Multiple High-Scoring Segments in Molecular Sequences. Proc Natl Acad Sci USA 90:5873-5877.
Karniski L P (1989) Activation of Cl⁻/OH⁻ Exchange by Parachloromercuribenzoic Acid in Rabbit Renal Brush-Border Membranes. J Membr Biol 112:59-66.
Karniski L P & Aronson P S (1987) Anion Exchange Pathways for Cl⁻ Transport in Rabbit Renal Microvillus Membranes. Am J Physiol 253:F513-521.
Karniski L P, Lotscher M, Fucentese M, Hilfiker H, Biber J & Murer H (1998a) Immunolocalization of Sat-1 Sulfate/Oxalate/Bicarbonate Anion Exchanger in the Rat Kidney. Am J Physiol 275:F79-87.
Karniski L P, Lotscher M, Fucentese M, Hilfiker H, Biber J & Murer H (1998b) Immunolocalization of Sat-1 Sulfate/Oxalate/Bicarbonate Anion Exchanger in the Rat Kidney. Am J Physiol 275:F79-87.
Katzung B G (2001) Basic & Clinical Pharmacology, 8th ed. Lange Medical Books/McGraw-Hill Medical Pub. Division, New York.
Kere J, Lohi H & Hoglund P (1999) Genetic Disorders of Membrane Transport III. Congenital Chloride Diarrhea. Am J Physiol 276:G7-G13.
Kestila M, Lenkkeri U, Mannikko M, Lamerdin J, McCready P, Putaala H, Ruotsalainen V, Morita T, Nissinen M, Herva R, Kashtan C E, Peltonen L, Holmberg C, Olsen A & Tryggvason K (1998) Positionally Cloned Gene for a Novel Glomerular Protein—Nephrin—Is Mutated in Congenital Nephrotic Syndrome. Mol Cell 1:575-582.
Khurana O K, Coupland L A, Shelden M C & Howitt S M (2000) Homologous Mutations in Two Diverse Sulphate Transporters Have Similar Effects. FEBS Lett 477:118-122.
Kimura S H, Tsuruga H, Yabuta N, Endo Y & Nojima H (1997) Structure, Expression, and Chromosomal Localization of Human Gak. Genomics 44:179-187.
Knauf F, Yang C L, Thomson R B, Mentone S A, Giebisch G & Aronson P S (2001) Identification of a Chloride-Formate Exchanger Expressed on the Brush Border Membrane of Renal Proximal Tubule Cells. Proc Natl Acad Sci USA 98:9425-9430.
Kozak M (1996) Interpreting cDNA Sequences: Some Insights from Studies on Translation. Mamm Genome 7:563-574.
Kreegipuu A, Blom N & Brunak S (1999) Phosphobase, a Database of Phosphorylation Sites: Release 2.0. Nucleic Acids Res 27:237-239.
Krick W, Dolle A, Hagos Y & Burckhardt G (1998) Characterization of the Chloride Conductance in Porcine Renal Brush-Border Membrane Vesicles. Pflugers Arch 435:415-421.
Kuo S M & Aronson P S (1988) Oxalate Transport Via the Sulfate/Hco3 Exchanger in Rabbit Renal Basolateral Membrane Vesicles. J Biol Chem 263:9710-9717.
Kurtz I, Nagami G, Yanagawa N, Li L, Emmons C & Lee 1 (1994) Mechanism of Apical and Basolateral Na⁺-lndependent Cl⁻/Base Exchange in the Rabbit Superficial Proximal Straight Tubule. J Clin Invest 94:173-183.
Kyte J & Doolittle R F (1982) A Simple Method for Displaying the Hydropathic Character of a Protein. J Mol Biol 157:105-132.
Labat-Moleur F, Steffan A M, Brisson C, Perron H, Feugeas 0, Furstenberger P, Oberling F, Brambilla E & Behr J P (1996) An Electron Microscopy Study into the Mechanism of Gene Transfer with Lipopolyamines. Gene Ther 3:1010-1017.
Landegren U, Nilsson M & Kwok P Y (1998) Reading Bits of Genetic Information: Methods for Single-Nucleotide Polymorphism Analysis. Genome Res 8:769-776.
Lasic D D & Martin F J (1995) Stealth® Liposomes. CRC Press, Boca Raton, Fla., United States of America.
Law B (1996) Immunoassay: A Practical Guide. Taylor & Francis, London/Bristol, Pa., United States of America.
Lee A, Beck L, Brown R J & Markovich D (1999a) Identification of a Mammalian Brain Sulfate Transporter. Biochem Biophys Res Commun 263:123-129.
Lee M C, Penland C M, Widdicombe J H & Wine J J (1998) Evidence That Calu-3 Human Airway Cells Secrete Bicarbonate. Am J Physiol 274:L450-453.
Lee M G, Choi J Y, Luo X, Strickland E, Thomas P J & Muallem S (1999b) Cystic Fibrosis Transmembrane Conductance Regulator Regulates Luminal Cl⁻/HCO₃⁻ Exchange in Mouse Submandibular and Pancreatic Ducts. J Biol Chem 274:14670-14677.
Li J J & Herskowitz 1 (1993) Isolation of ORC6, a Component of the Yeast Origin Recognition Complex by a One-Hybrid System. Science 262:1870-1874.
Li K, Christiano A M, Copeland N G, Gilbert D J, Chu M L, Jenkins N A & Uitto J (1993) cDNA Cloning and Chromosomal Mapping of the Mouse Type VII Collagen Gene (Col7a1): Evidence for Rapid Evolutionary Divergence of the Gene. Genomics 16:733-739.
Liddell E & Weeks 1 (1995) Antibody Technology. Bios Scientific Publishers, Oxford, United Kingdom.
Liman E R, Tytgat J & Hess P (1992) Subunit Stoichiometry of a Mammalian K⁺ Channel Determined by Construction of Multimeric cDNAs. Neuron 9:861-871.
Lohi H, Kujala M, Kerkela E, Saarialho-Kere U, Kestila M & Kere J (2000) Mapping of Five New Putative Anion Transporter Genes in Human and Characterization of SLC26A6, a Candidate Gene for Pancreatic Anion Exchanger. Genomics 70:102-112.
Low I, Friedrich T & Burckhardt G (1984) Properties of an Anion Exchanger in Rat Renal Basolateral Membrane Vesicles. Am J Physiol 246:F334-342.
Luo Y, Vijaychander S, Stile J & Zhu L (1996) Cloning and Analysis of DNA-Binding Proteins by Yeast One-Hybrid and One-Two-Hybrid Systems. Biotechniques 20:564-568.
Maalouf G J, Xu W, Smith T F & Mohr S C (1998) Homology Model for the Ligand-Binding Domain of the Human Estrogen Receptor. J Biomol Struct Dyn 15:841-851.
Mak P, McDonnell DP, Weigel N L, Schrader W T & O'Malley B W (1989) Expression of Functional Chicken Oviduct Progesterone Receptors in Yeast (Saccharomyces Cerevisiae). J Biol Chem 264:21613-21618.
Makalowski, W. & Boguski, M.S. Evolutionary parameters of the transcribed mammalian genome: an analysis of 2,820 orthologous rodent and human sequences. Proc Natl Acad Sci USA 95:9407-12 (1998).
Malmquist (1993) Nature 361:186-187.
Manome Y, Abe M, Hagen M F, Fine H A & Kufe D W (1994) Enhancer Sequences of the DF3 Gene Regulate Expression of the Herpes Simplex Virus Thymidine Kinase Gene and Confer Sensitivity of Human Breast Cancer Cells to Ganciclovir. Cancer Res 54:5408-5413.
Manson M M (1992) Immunochemical Protocols. Humana Press, Totowa, N.J., United States of America.
Mansoura M K, Biwersi J, Ashlock M A & Verkman A S (1999) Fluorescent Chloride Indicators to Assess the Efficacy of CFTR cDNA Delivery. Hum Gene Ther 10:861-875.
Marin M, Noel D & Piechaczyk M (1997) Towards Efficient Cell Targeting by Recombinant Retroviruses. Mol Med Today 3:396-403.
Markovich D & James K M (1999) Heavy Metals Mercury, Cadmium, and Chromium Inhibit the Activity of the Mammalian Liver and Kidney Sulfate Transporter Sat-1. Toxicol Appl Pharmacol 154:181-187.
Maruyama-Tabata H, Harada Y, Matsumura T, Satoh E, Cui F, lwai M, Kita M, Hibi S, Imanishi J, Sawada T & Mazda 0 (2000) Effective Suicide Gene Therapy in Vivo by EBV-Based Plasmid Vector Coupled with Polyamidoamine Dendrimer. Gene Ther 7:53-60.
Masereeuw R, Russel F G & Miller D S (1996) Multiple Pathways of Organic Anion Secretion in Renal Proximal Tubule Revealed by Confocal Microscopy. Am J Physiol 271:F1173-1182.
Masereeuw R, Moons M M, Toomey B H, Russel F G & Miller D S (1999) Active Lucifer Yellow Secretion in Renal Proximal Tubule: Evidence for Organic Anion Transport System Crossover. J Pharmacol Exp Ther 289:1104-1111.
Masseyeff R F, Albert WHW & Staines N (1993) Methods of Immunological Analysis. VCH Verlagsgesellschaft VCH Publishers, Weinheim (Federal Republic of Germany)/N.Y.
Michalk D V, Leitz R & Rauch J (1990) Influence of Dietary Sulfur-Containing Amino Acids on Renal Function and Metabolism of Calcium and Taurine in Chronic Renal Failure. Prog Clin Biol Res 351:451-462.
Miller D S, Letcher S & Barnes D M (1996) Fluorescence Imaging Study of Organic Anion Transport from Renal Proximal Tubule Cell to Lumen. Am J Physiol 271:F508-520.
Miura K, Himeno S, Koide N & Imura N (2000) Effects of Methylmercury and Inorganic Mercury on the Growth of Nerve Fibers in Cultured Chick Dorsal Root Ganglia. Tohoku J Exp Med 192:195-210.
Mohapatra N K, Cheng P W, Parker J C, Paradiso A M, Yankaskas J R, Boucher R C & Boat T F (1993) Sulfate Concentrations and Transport in Human Bronchial Epithelial Cells. Am J Physiol 264:C1231-1237.
Moseley R H, Hoglund P, Wu G D, Silberg D G, Haila S, de la Chapelle A, Holmberg C & Kere J (1999) Downregulated in Adenoma Gene Encodes a Chloride Transporter Defective in Congenital Chloride Diarrhea. Am J Physiol 276:G 185-192.
Needleman S B & Wunsch C D (1970) A General Method Applicable to the Search for Similarities in the Amino Acid Sequence of Two Proteins. J Mol Biol 48:443-453.
Neri D, Carnemolla B, Nissim A, Leprini A, Querze G, Balza E, Pini A, Tarli L, Halin C, Neri P, Zardi L & Winter G (1997) Targeting by Affinity-Matured Recombinant Antibody Fragments of an Angiogenesis Associated Fibronectin Isoform. Nat Biotechnol 15:1271-1275.
Newbury-Ecob R (1998) Atelosteogenesis Type 2. J Med Genet 35:49-53.
Nickerson D A, Kaiser R, Lappin S, Stewart J, Hood L & Landegren U (1990) Automated DNA Diagnostics Using an ELISA-Based Oligonucleotide Ligation Assay. Proc Natl Acad Sci USA 87:8923-8927.
Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.
Orita M, Iwahana H, Kanazawa H, Hayashi K & Sekiya T (1989) Detection of Polymorphisms of Human DNA by Gel Electrophoresis as Single-Strand Conformation Polymorphisms. Proc Natl Acad Sci USA 86:2766-2770.
Park J W, Hong K, Kirpotin D B, Meyer 0, Papahadjopoulos D & Benz C C (1997) Anti-Her2 Immunoliposomes for Targeted Therapy of Human Tumors. Cancer Lett 118:153-160.
Pasqualini R, Koivunen E & Ruoslahti E (1997) Alpha V Integrins as Receptors for Tumor Targeting by Circulating Ligands. Nat Biotechnol 15:542-546.
Pearson W R & Lipman D J (1988) Improved Tools for Biological Sequence Comparison. Proc Natl Acad Sci USA 85:2444-2448.
Pritchard J B (1987) Sulfate-Bicarbonate Exchange in Brush-Border Membranes from Rat Renal Cortex. Am J Physiol 252:F346-356.
Pritchard J B & Renfro J L (1983) Renal Sulfate Transport at the Basolateral Membrane Is Mediated by Anion Exchange. Proc Natl Acad Sci USA 80:2603-2607.
Pritchard J B & Miller D S (1993) Mechanisms Mediating Renal Secretion of Organic Anions and Cations. Physiol Rev 73:765-796.
Quandt K, Frech K, Karas H, Wingender E & Werner T (1995) Matind and Matinspector: New Fast and Versatile Tools for Detection of Consensus Matches in Nucleotide Sequence Data. Nucleic Acids Res 23:4878-4884.
Remington J P, Osol A, Anderson J T, Hoover J E & Skolaut M W (1975) Remington's Pharmaceutical Sciences, 15th ed. Mack Pub. Co., Easton, Pa., United States of America.
Roberts L (1991) GRAIL Seeks out Genes Buried in DNA Sequence. Science 254:805.
Romero M F, Hediger M A, Boulpaep E L & Boron W F (1997) Expression Cloning and Characterization of a Renal Electrogenic Na⁺/HCO₃⁻ Cotransporter. Nature 387:409-413
Romero M F, Fong P, Berger U V, Hediger M A & Boron W F (1998) Cloning and Functional Expression of RNBC, an Electrogenic Na(⁺)—HCO₃⁻ Cotransporter from Rat Kidney. Am J Physiol 274:F425-432.
Romero M F, Henry D, Nelson S, Harte P J, Dillon A K & Sciortino C M (2000) Cloning and Characterization of a Na⁺-Driven Anion Exchanger (NDAE1). A New Bicarbonate Transporter. J Biol Chem 275:24552-24559.
Rose M & Botstein D (1983) Construction and Use of Gene Fusions to Lacz (Beta-Galactosidase) That Are Expressed in Yeast. Methods Enzymol 101:167-180.
Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.
Royaux I E, Wall S M, Karniski L P, Everett L A, Suzuki K, Knepper M A & Green E D (2001) Pendrin, Encoded by the Pendred Syndrome Gene, Resides in the Apical Region of Renal Intercalated Cells and Mediates Bicarbonate Secretion. Proc Natl Acad Sci USA 98:4221-4226.
Saier M H, Jr., Eng B H, Fard S, Garg J, Haggerty D A, Hutchinson W J, Jack D L, Lai E C, Liu H J, Nusinew D P, Omar A M, Pao S S, Paulsen I T, Quan J A, Sliwinski M, Tseng T T, Wachi S & Young G B (1999) Phylogenetic Characterization of Novel Transport Protein Families Revealed by Genome Analyses. Biochim Biophys Acta 1422:1-56
Saltzman W M & Fung L K (1997) Polymeric Implants for Cancer Chemotherapy. Adv Drug Deliv Rev 26:209-230.
Sambrook et al. e (1989) Molecular Cloning: A Laboratory Manual. Cold Spring Harbor Laboratory Press, Cold Spring Harbor, N.Y.
Saqi M A, Wild D L & Hartshorn M J (1999) Protein Analyst—a Distributed Object Environment for Protein Sequence and Structure Analysis. Bioinformatics 15:521-522.
Satoh H, Susaki M, Shukunami C, lyama K, Negoro T & Hiraki Y (1998) Functional Analysis of Diastrophic Dysplasia Sulfate Transporter. Its Involvement in Growth Regulation of Chondrocytes Mediated by Sulfated Proteoglycans. J Biol Chem 273:12307-12315.
Schneider C H & Eberle A N (1993) Peptides, 1992: Proceedings of the Twenty-Second European Peptide Symposium. September 13-19, 1992. Interlaken. Switzerland. Escom, Leiden.
Schröder E & Lübke K (1965) The Peptides. Academic Press, New York.
Schug J & Overton G (1997) Tess: Transcription Element Search Software on the Www. Technical Report CBIL-TR-1997-1001-V0.0. Computational Biology and Informatics Laboratory, School of Medicine, University of Pennsylvania, United States of America.
Scott D A & Karniski L P (2000) Human Pendrin Expressed in Xenopus Laevis Oocytes Mediates Chloride/Formate Exchange. Am J Physiol Cell Physiol 278:C207-211.
Scott D A, Wang R, Kreman T M, Sheffield V C & Karniski L P (1999) The Pendred Syndrome Gene Encodes a Chloride-Iodide Transport Protein. Nat Genet 21:440-443.
Senekjian H O & Weinman E J (1982) Oxalate Transport by Proximal Tubule of the Rabbit Kidney. Am J Physiol 243:F271-275.
Siebens A W & Boron W F (1989) Depolarization-Induced Alkalinization in Proximal Tubules. II. Effects of Lactate and Sits. Am J Physiol 256:F354-365.
Silberg D G, Wang W, Moseley R H & Traber P G (1995) The Down Regulated in Adenoma (DRA) Gene Encodes an Intestine-Specific Membrane Sulfate Transport Protein. J Biol Chem 270:11897-11902.
Silhavy T J, Berman M L, Enquist L W & Cold Spring Harbor Laboratory. (1984) Experiments with Gene Fusions. Cold Spring Harbor Laboratory, Cold Spring Harbor, N.Y.
Singh H, Clerc R G & LeBowitz JH (1989) Molecular Cloning of Sequence-Specific DNA Binding Proteins Using Recognition Site Probes. Biotechniques 7:252-261.
Smith T F & Waterman M (1981) Comparison of Biosequences. Adv Appl Math 2:482-489.
Soleimani M, Greeley T, Petrovic S, Wang Z, Amial H, Kopp P & Burnham C E (2001) Pendrin: An Apical Cl⁻/OH⁻/HCO₃⁻ Exchanger in the Kidney Cortex. Am J Physiol Renal Physiol 280: F356-364.
Speight T M, Holford NHG & Avery G S (1997) Avery's Drug Treatment: A Guide to the Properties, Choice. Therapeutic Use and Economic Value of Drugs in Disease Management, 4^{th} ed. Adis International, Auckland/Philadelphia, Pa., United States of America.
Stoneking M, Hedgecock D, Higuchi R G, Vigilant L & Erlich H A (1991) Population Variation of Human mtDNA Control Region Sequences Detected by Enzymatic Amplification and Sequence-Specific Oligonucleotide Probes. Am J Hum Genet 48:370-382.
Superti-Furga A, Hastbacka J, Wilcox W R, Cohn D H, van der Harten H J, Rossi A, Blau N, Rimoin D L, Steinmann B, Lander E S & Gitzelmann R (1996) Achondrogenesis Type IB Is Caused by Mutations in the Diastrophic Dysplasia Sulphate Transporter Gene. Nat Genet 12:100-102.
Tallgren L G (1980) Inorganic Sulphates in Relation to the Serum Thyroxine Level and in Renal Failure. Acta Med Scand Suppl 640:1-100.
Talor Z, Gold R M, Yang W C & Arruda J A (1987) Anion Exchanger Is Present in Both Luminal and Basolateral Renal Membranes. Eur J Biochem 164:695-702.
Tam P, Monck M, Lee D, Ludkovski 0, Leng E C, Clow K, Stark H, Scherrer P, Graham R W & Cullis P R (2000) Stabilized Plasmid-Lipid Particles for Systemic Gene Therapy. Gene Ther 7:1867-1874.
Tijssen (1993) Laboratory Techniques in Biochemistry and Molecular Biology-Hybridization with Nucleic Acid Probes. Elsevier, New York.
Tsuganezawa H, Kobayashi K, lyori M, Araki T, Koizumi A, Watanabe S, Kaneko A, Fukao T, Monkawa T, Yoshida T, Kim D K, Kanai Y, Endou H, Hayashi M & Saruta T (2001) A New Member of the HCO₃(−) Transporter Superfamily Is an Apical Anion Exchanger of Beta-Intercalated Cells in the Kidney. J Biol Chem 276:8180-8189.
Uberbacher E C, Xu Y & Mural R J (1996) Discovering and Understanding Genes in Human DNA Sequence Using GRAIL. Methods Enzymol 266:259-281.
U.S. Pat. No. 4,196,265
U.S. Pat. No. 4,235,871
U.S. Pat. No. 4,455,842
U.S. Pat. No. 4,551,482
U.S. Pat. No. 4,554,101
U.S. Pat. No. 4,736,866
U.S. Pat. No. 4,946,778
U.S. Pat. No. 5,011,634
U.S. Pat. No. 5,091,513
U.S. Pat. No. 5,111,867
U.S. Pat. No. 5,132,405
U.S. Pat. No. 5,162,215
U.S. Pat. No. 5,223,409
U.S. Pat. No. 5,260,203
U.S. Pat. No. 5,264,563
U.S. Pat. No. 5,489,742
U.S. Pat. No. 5,490,840
U.S. Pat. No. 5,498,538
U.S. Pat. No. 5,510,103
U.S. Pat. No. 5,550,316
U.S. Pat. No. 5,573,933
U.S. Pat. No. 5,574,172
U.S. Pat. No. 5,629,145
U.S. Pat. No. 5,632,991
U.S. Pat. No. 5,614,396
U.S. Pat. No. 5,625,125
U.S. Pat. No. 5,648,061
U.S. Pat. No. 5,650,489
U.S. Pat. No. 5,651,991
U.S. Pat. No. 5,667,988
U.S. Pat. No. 5,677,427
U.S. Pat. No. 5,688,931
U.S. Pat. No. 5,702,892
U.S. Pat. No. 5,714,166
U.S. Pat. No. 5,741,957
U.S. Pat. No. 5,738,996
U.S. Pat. No. 5,747,334
U.S. Pat. No. 5,756,291
U.S. Pat. No. 5,776,859
U.S. Pat. No. 5,780,225
U.S. Pat. No. 5,780,242
U.S. Pat. No. 5,786,387
U.S. Pat. No. 5,824,483
U.S. Pat. No. 5,834,228
U.S. Pat. No. 5,840,479
U.S. Pat. No. 5,849,877
U.S. Pat. No. 5,855,900
U.S. Pat. No. 5,858,410
U.S. Pat. No. 5,858,670
U.S. Pat. No. 5,872,011
U.S. Pat. No. 5,892,019
U.S. Pat. No. 5,922,254
U.S. Pat. No. 5,922,356
U.S. Pat. No. 5,922,545
U.S. Pat. No. 5,948,635
U.S. Pat. No. 5,948,647
U.S. Pat. No. 5,948,767
U.S. Pat. No. 5,985,279
U.S. Pat. No. 5,994,392
U.S. Pat. No. 6,054,561
U.S. Pat. No. 6,056,938
U.S. Pat. No. 6,061,581
U.S. Pat. No. 6,057,098
U.S. Pat. No. 6,071,890
U.S. Pat. No. 6,087,111
U.S. Pat. No. 6,090,925
U.S. Pat. No. 6,106,866
U.S. Pat. No. 6,107,059
U.S. Pat. No. 6,120,787
U.S. Pat. No. 6,127,339
U.S. Pat. No. 6,132,766
U.S. Pat. No. 6,140,123
U.S. Pat. No. 6,156,511
U.S. Pat. No. 6,168,912
U.S. Pat. No. 6,174,708
U.S. Pat. No. 6,176,089
U.S. Pat. No. 6,180,084
U.S. Pat. No. 6,180,348
U.S. Pat. No. 6,190,700
U.S. Pat. No. 6,197,333
U.S. Pat. No. 6,200,598
U.S. Pat. No. 6,210,707
U.S. Pat. No. 6,214,553
U.S. Pat. No. 6,217,886
U.S. Pat. No. 6,221,958
U.S. Pat. No. 6,238,704
U.S. Pat. No. 6,238,705
U.S. Pat. No. 6,245,740
U.S. Pat. No. 6,262,127
U.S. Pat. No. 6,267,981
U.S. Pat. No. 6,287,587
U.S. Pat. No. 6,296,832
U.S. Pat. No. 6,296,842
U.S. Pat. No. 6,312,713
U.S. Pat. No. 6,335,035
Vacca C V, Hines J D & Hall P W, 3rd (1986) The Proteinuria of Industrial Lead Intoxication. Environ Res 41:440-446.
Verkman A S (1990) Development and Biological Applications of Chloride-Sensitive Fluorescent Indicators. Am J Physiol 259:C375-388.
Vidal M, Brachmann R K, Fattaey A, Harlow E & Boeke J D (1996) Reverse Two-Hybrid and One-Hybrid Systems to Detect Dissociation of Protein-Protein and DNA-Protein Interactions. Proc Natl Acad Sci USA 93:10315-10320.
Waldegger S, Moschen I, Ramirez A, Smith R J, Ayadi H, Lang F & Kubisch C (2001) Cloning and Characterization of SLC26A6, a Novel Member of the Solute Carrier 26 Gene Family. Genomics 72:43-50.
Walker M R & Rapley R (1993) Molecular and Antibody Probes in Diagnosis. Wiley, Chichester; N.Y.
Wang D G, Fan J B, Siao C J, Berno A, Young P, Sapolsky R, Ghandour G, Perkins N, Winchester E, Spencer J, Kruglyak L, Stein L, Hsie L, Topaloglou T, Hubbell E, Robinson E, Mittmann M, Morris M S, Shen N, Kilburn D, Rioux J, Nusbaum C, Rozen S, Hudson T J, Lander E S & et al. (1998) Large-Scale Identification, Mapping, and Genotyping of Single-Nucleotide Polymorphisms in the Human Genome. Science 280:1077-1082.
Wang T, Giebisch G & Aronson P S (1992) Effects of Formate and Oxalate on Volume Absorption in Rat Proximal Tubule. Am J Physiol 263:F37-42.
Wang T, Yang C L, Abbiati T, Shull G E, Giebisch G & Aronson P S (2001) Essential Role of NHE3 in Facilitating Formate-Dependent NaCl Absorption in the Proximal Tubule. Am J Physiol Renal Physiol 281:F288-292.
Wang Z, Petrovic S, Mann E & Soleimani M (2002) Identification of an Apical Cl(−)/HCO Exchanger in the Small Intestine. Am J Physiol Gastrointest Liver Physiol 282:G573-G579.
Wiedemann M & Trueb B (2001) The Mouse FGFRL1 Gene Coding for a Novel FGF Receptor-Like Protein. Biochim Biophys Acta 1520:247-250.
Worrall T A, Cotter R J & Woods A S (1998) Purification of Contaminated Peptides and Proteins on Synthetic Membrane Surfaces for Matrix-Assisted Laser Desorption/Ionization Mass Spectrometry. Anal Chem 70:750-756.
Wyckoff H W, Hirs CHW & Timasheff S N (1985) Diffraction Methods for Biological Macromolecules. Academic Press, Orlando, Fla., United States of America.
Xu Y, Mural R, Shah M & Uberbacher E (1994) Recognizing Exons in Genomic Sequence Using GRAIL II. Genet Eng 16:241-253.
Yuan B, Thomas J P, von Kodolitsch Y & Pyeritz R E (1999) Comparison of Heteroduplex Analysis, Direct Sequencing, and Enzyme Mismatch Cleavage for Detecting Mutations in a Large Gene, Fbn1. Hum Mutat 14:440-446.
Zheng J, Shen W, He D Z, Long K B, Madison L D & Dallos P (2000) Prestin Is the Motor Protein of Cochlear Outer Hair Cells. Nature 405:149-155.

It will be understood that various details of the invention can be changed without departing from the scope of the invention. Furthermore, the foregoing description is for the purpose of illustration only, and not for the purpose of limitation—the invention being defined by the claims appended hereto.

What is claimed is:

1. An isolated polypeptide comprising a functional SLC26A6 polypeptide.

2. The functional SLC26A6 polypeptide of claim 1 comprising:
(a) a polypeptide encoded by a nucleic acid of any one of odd-numbered SEQ ID NOs:1-7;
(b) a polypeptide encoded by a nucleic acid substantially identical to any one of odd-numbered SEQ ID NOs:1-7;
(c) a polypeptide comprising an amino acid sequence of any one of even-numbered SEQ ID NOs:2-8; or
(d) a polypeptide substantially identical to any one of even-numbered SEQ ID NOs:2-8.

3. The functional SLC26A6 polypeptide of claim 1, wherein the SLC26A6 polypeptide is encoded by an isolated nucleic acid segment selected from the group consisting of:
(a) an isolated nucleic acid molecule encoding a polypeptide of any one of even-numbered SEQ ID NOs:2-8;
(b) an isolated nucleic acid molecule of any one of odd-numbered SEQ ID NOs:1-7;
(c) an isolated nucleic acid molecule which hybridizes to a nucleic acid sequence of any one of odd-numbered SEQ ID NOs:1-7 under wash stringency conditions represented by a wash solution having less than about 200 mM salt concentration and a wash temperature of greater than about 45° C., and which encodes a functional SLC26A6 polypeptide; and
(d) an isolated nucleic acid molecule differing by at least one functionally equivalent codon from the isolated nucleic acid molecule of one of (a), (b), and (c) above in nucleic acid sequence due to the degeneracy of the genetic code, and which encodes a functional SLC26A6 polypeptide encoded by the isolated nucleic acid of one of (a), (b), and (c) above.

4. The functional SLC26A6 polypeptide of claim 1, wherein the functional property comprises Cl⁻-fomate exchange.

5. The functional SLC26A6 polypeptide of claim 1, wherein the functional property comprises Cl⁻—Cl⁻ exchange.

6. The functional SLC26A6 polypeptide of claim 1, wherein the functional property comprises SO₄²⁻ exchange.

7. The functional SLC26A6 polypeptide of claim 1, wherein the functional property comprises Cl⁻-oxalate exchange.

8. The functional SLC26A6 polypeptide of claim 8, wherein the functional property comprises Cl⁻-base exchange.

9. The functional SLC26A6 polypeptide of claim 8, wherein the base comprises HCO₃⁻.

10. The functional SLC26A6 polypeptide of claim 1, wherein the Cl⁻-base exchange comprises electrogenic transport.

11. A system for recombinant expression of a functional SLC26A6 polypeptide, the system comprising:
(a) a functional SLC26A6 polypeptide; and
(b) a host cell comprising the functional SLC26A6 polypeptide.

12. The system of claim 11, wherein the host cell comprises a mammalian cell.

13. The system of claim 12, wherein the mammalian cell comprises a human cell.

14. An isolated nucleic acid encoding a human SLC26A6a polypeptide.

15. The isolated nucleic acid of claim 14, further comprising a nucleic acid encoding a polypeptide of SEQ ID NO:2.

16. The isolated nucleic acid of claim 14, further comprising a nucleic acid of SEQ ID NO:1.

17. An isolated human SLC26A6a polypeptide.

18. The isolated human SLC26A6a polypeptide of claim 17, further comprising:
(a) a polypeptide of SEQ ID NO:2; or
(b) a polypeptide encoded by a nucleic acid molecule of SEQ ID NO:1.

19. A system for recombinant expression of a human SLC26A6a polypeptide, the system comprising:
(a) a human SLC26A6a polypeptide; and
(b) a host cell comprising the human SLC26A6a polypeptide.

20. An isolated nucleic acid encoding a mouse SLC26A6 polypeptide.

21. The isolated nucleic acid of claim 20, further comprising a nucleic acid encoding a polypeptide of SEQ ID NO:6 or 8.

22. The isolated nucleic acid of claim 20, further comprising a nucleic acid of SEQ ID NO:5 or 7.

23. An isolated mouse SLC26A6 polypeptide.

24. The isolated mouse SLC26A6 polypeptide of claim 23, further comprising:
(a) a polypeptide of SEQ ID NO:6 or 8; or
(b) a polypeptide encoded by a nucleic acid molecule of SEQ ID NO:5 or 7.

25. A system for recombinant expression of a mouse SLC26A6 polypeptide, the system comprising:
(a) a mouse SLC26A6 polypeptide; and
(b) a host cell comprising the mouse SLC26A6 polypeptide.

26. A method for identifying a modulator of a SLC26A6 polypeptide, the method comprising:
(a) providing a recombinant expression system whereby a functional SLC26A6 polypeptide is expressed in a host cell;
(b) providing a test substance to the system of (a);
(c) assaying a level or quality of SLC26A6 function in the presence of the test substance;
(d) comparing the level or quality of SLC26A6 function in the presence of the test substance with a control level or quality of SLC26A6 function; and
(e) identifying a test substance as an anion transport modulator by determining a level or quality of SLC26A6 function in the presence of the test substance as significantly changed when compared to a control level or quality of SLC26A6 function.

27. The method of claim 26, wherein the functional SLC26A6 polypeptide comprises:
(a) a polypeptide encoded by a nucleic acid of any one of odd-numbered SEQ ID NOs:1-7;
(b) a polypeptide encoded by a nucleic acid substantially identical to any one of odd-numbered SEQ ID NOs:1-7;
(c) a polypeptide comprising an amino acid sequence of any one of even-numbered SEQ ID NOs:2-8; or
(d) a polypeptide substantially identical to any one of even-numbered SEQ ID NOs:2-8.

28. The method of claim 26, wherein the functional SLC26A6 polypeptide is encoded by an isolated nucleic acid segment selected from the group consisting of:
(a) an isolated nucleic acid molecule encoding a polypeptide of any one of even-numbered SEQ ID NOs:2-8;
(b) an isolated nucleic acid molecule of any one of odd-numbered SEQ ID NOs:1-7;
(c) an isolated nucleic acid molecule which hybridizes to a nucleic acid sequence of any one odd-numbered SEQ ID NOs:1-7 under wash stringency conditions represented by a wash solution having less than about 200 mM salt concentration and a wash temperature of greater than about 45° C., and which encodes a functional SLC26A6 polypeptide; and
(c) an isolated nucleic acid molecule differing by at least one functionally equivalent codon from the isolated nucleic acid molecule of one of (a), (b), and (c) above in nucleic acid sequence due to the degeneracy of the genetic code, and which encodes a functional SLC26A6 polypeptide encoded by the isolated nucleic acid of one of (a), (b), and (c) above.

29. The method of claim 26, wherein the host cell comprises a mammalian cell.

30. The method of claim 29, wherein the mammalian cell comprises a human cell.

31. The method of claim 26, wherein the SLC26A6 function comprises Cl⁻-fomate exchange.

32. The method of claim 26, wherein the SLC26A6 function comprises Cl⁻—Cl⁻ exchange.

33. The method of claim 26, wherein the SLC26A6 function comprises SO₄²⁻ exchange.

34. The method of claim 26, wherein the SLC26A6 function comprises Cl⁻-oxalate exchange.

35. The method of claim 26, wherein the SLC26A6 function comprises Cl⁻-base exchange.

36. The method of claim 35, wherein the base comprises HCO₃⁻.

37. The method of claim 35, wherein the Cl⁻-base exchange comprises electrogenic transport.

38. An anion transporter modulator identified by the method of claim 26.

39. A method for modulating anion transport activity in a subject, the method comprising:
(a) preparing a composition comprising a modulator identified according to the method of claim 26, and a pharmaceutically acceptable carrier;
(b) administering an effective dose of the composition to a subject, whereby anion transport is altered in the subject.

40. The method of claim 39, wherein the subject is a mammal.

41. The method of claim 40, wherein the mammal is a human.

42. A method for identifying an anion exchanger modulator, the method comprising:
(a) exposing a SLC26A6 polypeptide to one or more test substances, wherein the SLC26A6 polypeptide comprises a mouse SLC26A6 polypeptide or a human SLC26A6a polypeptide;
(b) assaying binding of a test substance to the isolated SLC26A6 polypeptide; and
(c) selecting a candidate substance that demonstrates specific binding to the SLC26A6 polypeptide.

43. The method of claim 42, wherein the mouse SLC26A6 polypeptide comprises:
(a) a polypeptide of SEQ ID NO:6 or 8; or
(b) a polypeptide encoded by a nucleic acid molecule of SEQ ID NO:5 or 7.

44. The method of claim 42, wherein the mouse SLC26A6a polypeptide comprises:
(a) a polypeptide of SEQ ID NO:2; or
(b) a polypeptide encoded by a nucleic acid molecule of SEQ ID NO:1.

45. An anion transporter modulator identified by the method of claim 42.

46. A method for modulating anion transport activity in a subject, the method comprising:
(a) preparing a composition comprising a modulator identified according to the method of claim 42, and a pharmaceutically acceptable carrier;
(b) administering an effective dose of the composition to a subject, whereby SLC26 function is altered in the subject.

47. The method of claim 46, wherein the subject is a mammal.

48. The method of claim 47, wherein the subject is a human.

49. A method for modulating a SLC26A6 polypeptide in a subject, the method comprising administering an effective amount of a SLC26A6 modulator to the subject, wherein the SLC26A6 modulator comprises a pH modifier.

50. The method of claim 49, wherein the subject is a mammal.

51. The method of claim 50, wherein the mammal is a human.

52. An isolated nucleic acid encoding a mouse SLC26A1 polypeptide.

53. The isolated nucleic acid of claim 52, further comprising a nucleic acid encoding a polypeptide of SEQ ID NO:10.

54. The isolated nucleic acid of claim 52, further comprising a nucleic acid of SEQ ID NO:9.

55. An isolated mouse SLC26A1 polypeptide.

56. The isolated mouse SLC26A1 polypeptide of claim 55, further comprising:
(a) a polypeptide encoded by a nucleic acid of SEQ ID NO:9; or
(b) a polypeptide comprising an amino acid sequence of SEQ ID NO: 10.

57. The mouse SLC26A1 polypeptide of claim 55, further comprising a functional mouse SLC26A1 polypeptide.

58. A system for recombinant expression of a mouse SLC26A1 polypeptide, the system comprising:
(a) a mouse SLC26A1 polypeptide; and
(b) a host cell comprising the mouse SLC26A1 polypeptide.

59. A method for identifying a modulator of a SLC26A1 polypeptide, the method comprising:
(a) providing a recombinant expression system whereby a functional mouse SLC26A1 polypeptide is expressed in a host cell;
(b) providing a test substance to the system of (a);
(c) assaying a level or quality of SLC26A1 function in the presence of the test substance;
(d) comparing the level or quality of SLC26A1 function in the presence of the test substance with a control level or quality of SLC26A1 function; and
(e) identifying a test substance as an anion transport modulator by determining a level or quality of SLC26A1 function in the presence of the test substance as significantly changed when compared to a control level or quality of SLC26A1 function.

60. The method of claim 57, wherein the mouse SLC26A1 comprises:
(a) a polypeptide encoded by a nucleic acid of SEQ ID NO:9; or
(b) a polypeptide comprising an amino acid sequence of SEQ ID NO:10.

61. An anion transporter modulator identified by the method of claim 59.

62. A method for identifying an anion exchanger modulator, the method comprising:
(a) exposing an isolated mouse SLC26A1 polypeptide to one or more test substances;
(b) assaying binding of a test substance to the isolated mouse SLC26A1 polypeptide; and
(c) selecting a candidate substance that demonstrates specific binding to the SLC26A1 polypeptide.

63. The method of claim 62, wherein the mouse SLC26A1 comprises:
(a) a polypeptide encoded by a nucleic acid of SEQ ID NO:9; or
(b) a polypeptide comprising an amino acid sequence of SEQ ID NO:10.

64. An anion transporter modulator identified by the method of claim 62.

65. A method for activating a SLC26A1 polypeptide in a subject, the method comprising administering an effective amount of a SLC26A1 modulator to the subject, wherein the SLC26A1 modulator comprises an impermeant anion.

66. The method of claim 65, wherein the impermeant anion comprises Cl⁻ or formate.

67. A method for modulating a SLC26A2 polypeptide in a subject, the method comprising administering an effective amount of a SLC26A2 modulator to the subject, wherein the SLC26A2 modulator comprises a pH modifier.

68. An isolated nucleic acid encoding a porcine SLC26A6 polypeptide.

69. The isolated nucleic acid of claim 68, further comprising a nucleic acid encoding a polypeptide of SEQ ID NO:91.

70. The isolated nucleic acid of claim 68, further comprising a nucleic acid of SEQ ID NO:90.

71. An isolated porcine SLC26A6 polypeptide.

72. The isolated porcine SLC26A6 polypeptide of claim 71, further comprising:
(a) a polypeptide encoded by a nucleic acid of SEQ ID NO:90; or
(b) a polypeptide comprising an amino acid sequence of SEQ ID NO:91.

73. An isolated nucleic acid encoding a Xenopus laevis SLC26A6 polypeptide.

74. The isolated nucleic acid of claim 73, further comprising a nucleic acid encoding a polypeptide of SEQ ID NO:89.

75. The isolated nucleic acid of claim 73, further comprising a nucleic acid of SEQ ID NO:88.

76. An isolated Xenopus laevis SLC26A6 polypeptide.

77. The isolated Xenopus laevis SLC26A6 polypeptide of claim 76, further comprising:
(a) a polypeptide encoded by a nucleic acid of SEQ ID NO:88; or
(b) a polypeptide comprising an amino acid sequence of SEQ ID NO:89.

78. An isolated nucleic acid encoding a Xenopus laevis SLC26A1 polypeptide.

79. The isolated nucleic acid of claim 78, further comprising a nucleic acid encoding a polypeptide of SEQ ID NO:87.

80. The isolated nucleic acid of claim 78, further comprising a nucleic acid of SEQ ID NO:86.

81. An isolated Xenopus laevis SLC26A1 polypeptide.

82. The isolated Xenopus laevis SLC26A1 polypeptide of claim 81, further comprising:
(a) a polypeptide encoded by a nucleic acid of SEQ ID NO:86; or
(b) a polypeptide comprising an amino acid sequence of SEQ ID NO:87.

83. An isolated nucleic acid encoding a Xenopus laevis SLC26A4 polypeptide.

84. The isolated nucleic acid of claim 83, further comprising a nucleic acid encoding a polypeptide of one of SEQ ID NOs:81, 83, and 85.

85. The isolated nucleic acid of claim 83, further comprising a nucleic acid of one of SEQ ID NOs:80, 82, and 84.

86. An isolated Xenopus laevis SLC26A4 polypeptide.

87. The isolated Xenopus laevis SLC26A4 polypeptide of claim 86, further comprising:
(a) a polypeptide encoded by a nucleic acid of one of SEQ ID NOs:80, 82, and 84; or
(b) a polypeptide comprising an amino acid sequence of one of SEQ ID NOs:81, 83, and 85.
